(19) United States
(12) Patent Application Publication
(10) Pub. No.: US 2026/0049134 A1
(43) Pub. Date: Feb. 19, 2026

(54) RABBIT MONOCLONAL ANTIBODIES TARGETING MULTIPLE MYELOMA CELL SURFACE ANTIGENS

(71) Applicant: University of Florida Research Foundation, Incorporated, Gainesville, FL (US)

(72) Inventors: Christoph RADER, Jupiter, FL (US); Henry D. WILSON, Watertown, MA (US); Matthew CYR, Palm Beach, FL (US)

(21) Appl. No.: 19/102,298

(22) PCT Filed: Aug. 9, 2023
(86) PCT No.: PCT/US2023/071925

§ 371 Date: Feb. 7, 2025

Related U.S. Application Data
(60) Provisional application No. 63/397,277, filed on Aug. 11, 2022.

(51) Int. Cl. C07K 16/28

(52) U.S. Cl. CPC C07K 16/2803; C07K 2317/24; C07K 2317/92

(57) ABSTRACT

The invention provides antibodies, antibody fragments or antigen-binding fragments, as well as related antibody drug conjugates (ADCs) and chimeric antigen receptors (CARs), that specifically recognize a multiple myeloma cell surface antigen selected from PTPRG, CADM1, ICAM1, and GARS. Also provided in the invention are methods of using such antibodies in various diagnostic and therapeutic applications for hematologic malignancies including multiple myeloma and acute myeloid leukemia (AML).

### CROSS REFERENCE TO RELATED APPLICATIONS

This application claims the benefit of U.S. Provisional Application 63/397,277 filed Aug. 11, 2022, which is incorporated by reference in its entirety for all purposes.

### FEDERALLY SPONSORED RESEARCH OR DEVELOPMENT

This invention was made with government support under Grant Numbers R01 CA181258 and TL1 TR002551, awarded by the National Institutes of Health. The government has certain rights in the invention.

### REFERENCE TO A SEQUENCE LISTING

The Sequence Listing written in file 596602SEQLST.xml is 160,311 bytes, was created on Aug. 2, 2023, and is hereby incorporated by reference.

### BACKGROUND OF THE INVENTION

Multiple myeloma (MM) is a hematologic malignancy characterized by clonal expansion of malignant plasma cells that accumulate in the bone marrow. Bone pain, hypercalcemia, renal insufficiency, anemia, bone lesions, hyperviscosity, amyloidosis, fatigue, and recurrent infections, may occur as the disease progresses. Current treatments for MM include proteasome inhibitors, immunomodulatory drugs, and monoclonal antibodies, however drug resistance and systemic toxicity limit their usefulness in patients. [Yang, P et al., MedComm (2020). 2022 June; 3(2): e146].

Monoclonal antibody (mAb)-based biologics have revolutionized cancer therapy. Defining antibody-based cancer therapeutics by their ability to target tumor or tumor-associated cells through antibody-based recognition [1], 52 different such biologics have been approved by the Food and Drug Administration (FDA) and are on the market as of January 2023 (66). While the number of antibody-based cancer therapeutics has grown continuously over the past 25 years, there still is a strong bias with respect to their targets. In fact, 29 of these 52 biologics target one of only seven antigens, i.e. CD19 (7), HER2 (5), PD1 (4), PDL1 (3), CD20 (4), BCMA (3), and EGFR (3). Thus, only a small fraction of the ˜5,000 cell surface proteins found in humans have been targeted by antibody therapeutics, a number which neglects to capture the added diversity gained by mutations, alternative splicing, and post-translational modifications (PTMs). Furthermore, many antibody-based cancer therapeutics have side effects due to on-target-off-tissue toxicity, highlighting the need to find new targets with potentially improved therapeutic indices.

One powerful tool that has enabled discovery of clinically and commercially successful mAb-based biologics is phage display [2-4]. The simple biology of phagemid/phage systems allows for monovalent display of antibody fragments (e.g. Fabs) via fusion to the N-terminus of the minor capsid protein pIII (or a C-terminal fragment thereof) and the fishing out of antibodies with unique reactivity that can then be determined by sequencing the phagemid DNA. Phage display selection of antibody libraries has several advantages over the traditional animal immunization including lower cost, faster results, and circumvention of immune tolerance (67) Since many membrane proteins prove difficult to express recombinantly and purify, phage display strategies have been developed to select antibody libraries on the surface of whole cells where the target is in situ in its native conformation [5, 6]. Such selection strategies, referred to as whole-cell panning (WCP), have required creative solutions to avoid selecting antibodies that bind to all cells or common cell surface proteins including depletion with cells or membrane vesicles that lack the target of interest [7, 8].

Given the huge sequence and structural diversity in antibody fragment (e.g., Fab, scFv, or single domain) libraries and their facile in vitro selection by phage display, WCP also offers the opportunity for target-agnostic selection on whole cells to simultaneously identify both the antibody and the prospective antigen [5, 8, 9]. This unbiased antibody-driven drug and target discovery approach is not limited to validated cell surface proteins, and thus has the potential to identify unappreciated human cancer cell surface-ome components [10], such as cancer cell-specific isoforms, conformations [11], multiprotein complexes, PTMs including glycosylation [12], or surface RNAs [13]. However, technical aspects of phagemid-based phage display such as the carryover and competitive outgrowth of ‘bald’ phage that lack displayed antibody fragments have made the successful enrichment and isolation of cell surface binding antibodies challenging [14, 15].

Another problem common to all target-agnostic drug discovery efforts is the identification of the target. In the case of antibodies, immunoprecipitation (IP) is the traditional method of choice, yet it does not work universally and can require extensive optimization, making it non-ideal for the parallel deorphanization of multiple candidate mAbs. Protein arrays, in which individual membrane proteins are overexpressed on the cell surface, provide useful information for on- and off-target interactions but are expensive and limited to one or two cell types, which express endogenous surface proteins resulting in high background and the inability to identify specific antigens(23, 24).

Therefore, there is a need for antibodies to multiple myeloma cell surface antigens that can be used as a basis for the development of antibody-based targeted therapies of hematologic malignancies including multiple myeloma and acute myeloid leukemia (AML). There is also a need for additional diagnostic tools for detecting multiple myeloma cell surface antigen expression in hematologic malignancies including multiple myeloma and acute myeloid leukemia (AML), e.g. such as Western-blotting and or immunohistochemistry (IHC). The instant invention is directed to addressing these and other needs.

### SUMMARY OF THE INVENTION

In one aspect, the invention provides an isolated antibody that competes for binding to a multiple myeloma cell surface antigen with antibody HW-42, HW-70, HW-25, HW-1, HW-17, HW-8, HW-16, HW-45, HW-28, HW-56, HW-81, HW-97, HW-101, or HW-113. Some antibodies bind to the same epitope on a multiple myeloma cell surface antigen as antibody HW-42, HW-70, HW-25, HW-1, HW-17, HW-8, HW-16, HW-45, HW-28, HW-56, HW-81, HW-97, HW-101, or HW-113. Some antibodies bind a multiple myeloma cell surface antigen that is PTPRG, ICAM1, CADM1, or GARS.

In another aspect, the antibody specifically binds PTPRG and comprises three heavy chain CDRs and three light chain CDRs of antibody HW-42, wherein HW-42 is characterized by a mature heavy chain variable region having an amino acid sequence comprising SEQ ID NO:11 and a mature light chain variable region having an amino acid sequence comprising SEQ ID NO:12; comprises three heavy chain CDRs and three light chain CDRs of antibody HW-70, wherein HW-70 is characterized by a mature heavy chain variable region having an amino acid sequence comprising SEQ ID NO:21 and a mature light chain variable region having an amino acid sequence comprising SEQ ID NO:22; comprises three heavy chain CDRs and three light chain CDRs of antibody HW-25, wherein HW-25 is characterized by a mature heavy chain variable region having an amino acid sequence comprising SEQ ID NO:31 and a mature light chain variable region having an amino acid sequence comprising SEQ ID NO:32; comprises three heavy chain CDRs and three light chain CDRs of antibody HW-1, wherein HW-1 is characterized by a mature heavy chain variable region having an amino acid sequence comprising SEQ ID NO:41 and a mature light chain variable region having an amino acid sequence comprising SEQ ID NO:42; or comprises three heavy chain CDRs and three light chain CDRs of antibody HW-17, wherein HW-17 is characterized by a mature heavy chain variable region having an amino acid sequence comprising SEQ ID NO:51 and a mature light chain variable region having an amino acid sequence comprising SEQ ID NO:52.

In some antibodies, the CDRs are of a definition selected from the group consisting of Kabat, Chothia, Kabat/Chothia Composite, AbM, Contact, and IMGT.

In some such antibodies, the mature heavy chain variable region comprises the three IMGT heavy chain CDRs of SEQ ID NOs:15-17 and the mature light chain variable region comprises the three IMGT light chain CDRs of SEQ ID NOs:18-20; the mature heavy chain variable region comprises the three IMGT heavy chain CDRs of SEQ ID NOs:25-27 and the mature light chain variable region comprises the three IMGT light chain CDRs of SEQ ID NOs:28-30; the mature heavy chain variable region comprises the three IMGT heavy chain CDRs of SEQ ID NOs:35-37 and the mature light chain variable region comprises the three IMGT light chain CDRs of SEQ ID NOs:38-40; the mature heavy chain variable region comprises the three IMGT heavy chain CDRs of SEQ ID NOs:45-47 and the mature light chain variable region comprises the three IMGT light chain CDRs of SEQ ID NOs:48-50; or the mature heavy chain variable region comprises the three IMGT heavy chain CDRs of SEQ ID NOs:55-57 and the mature light chain variable region comprises the three IMGT light chain CDRs of SEQ ID NOs:58-60.

Some antibodies comprise a mature heavy chain variable region having an amino acid sequence at least 90% identical to SEQ ID NO:11, and a mature light chain variable region having an amino acid sequence at least 90% identical to SEQ ID NO:12; comprise a mature heavy chain variable region having an amino acid sequence at least 90% identical to SEQ ID NO:21, and a mature light chain variable region having an amino acid sequence at least 90% identical to SEQ ID NO:22; comprise a mature heavy chain variable region having an amino acid sequence at least 90% identical to SEQ ID NO:31, and a mature light chain variable region having an amino acid sequence at least 90% identical to SEQ ID NO:32; comprise a mature heavy chain variable region having an amino acid sequence at least 90% identical to SEQ ID NO:41, and a mature light chain variable region having an amino acid sequence at least 90% identical to SEQ ID NO:42; or comprise a mature heavy chain variable region having an amino acid sequence at least 90% identical to SEQ ID NO:51, and a mature light chain variable region having an amino acid sequence at least 90% identical to SEQ ID NO:52.

In some antibodies, the mature heavy chain variable region has an amino acid sequence of SEQ ID NO:11 and the mature light chain variable region has an amino acid sequence of SEQ ID NO:12; the mature heavy chain variable region has an amino acid sequence of SEQ ID NO:21 and the mature light chain variable region has an amino acid sequence of SEQ ID NO:22; the mature heavy chain variable region has an amino acid sequence of SEQ ID NO:31 and the mature light chain variable region has an amino acid sequence of SEQ ID NO:32; the mature heavy chain variable region has an amino acid sequence of SEQ ID NO:41 and the mature light chain variable region has an amino acid sequence of SEQ ID NO:42; or the mature heavy chain variable region has an amino acid sequence of SEQ ID NO:51 and the mature light chain variable region has an amino acid sequence of SEQ ID NO:52.

In another aspect, the antibody specifically binds GARS and comprises three heavy chain CDRs and three light chain CDRs of antibody HW-8, wherein HW-8 is characterized by a mature heavy chain variable region having an amino acid sequence comprising SEQ ID NO:61 and a mature light chain variable region having an amino acid sequence comprising SEQ ID NO:62; comprises three heavy chain CDRs and three light chain CDRs of antibody HW-16, wherein HW-16 is characterized by a mature heavy chain variable region having an amino acid sequence comprising SEQ ID NO:71 and a mature light chain variable region having an amino acid sequence comprising SEQ ID NO:72; comprises three heavy chain CDRs and three light chain CDRs of antibody HW-45, wherein HW-45 is characterized by a mature heavy chain variable region having an amino acid sequence comprising SEQ ID NO:81 and a mature light chain variable region having an amino acid sequence comprising SEQ ID NO:82; comprises three heavy chain CDRs and three light chain CDRs of antibody HW-28, wherein HW-28 is characterized by a mature heavy chain variable region having an amino acid sequence comprising SEQ ID NO:91 and a mature light chain variable region having an amino acid sequence comprising SEQ ID NO:92; comprises three heavy chain CDRs and three light chain CDRs of antibody HW-56, wherein HW-56 is characterized by a mature heavy chain variable region having an amino acid sequence comprising SEQ ID NO:101 and a mature light chain variable region having an amino acid sequence comprising SEQ ID NO:102; or comprises three heavy chain CDRs and three light chain CDRs of antibody HW-81, wherein HW-81 is characterized by a mature heavy chain variable region having an amino acid sequence comprising SEQ ID NO:111 and a mature light chain variable region having an amino acid sequence comprising SEQ ID NO:112.

In some such antibodies, the CDRs are of a definition selected from the group consisting of Kabat, Chothia, Kabat/Chothia Composite, AbM, Contact, and IMGT.

In some antibodies, the mature heavy chain variable region comprises the three IMGT heavy chain CDRs of SEQ ID NOs:65-67 and the mature light chain variable region comprises the three IMGT light chain CDRs of SEQ ID NOs:68-70; the mature heavy chain variable region comprises the three IMGT heavy chain CDRs of SEQ ID NOs:75-77 and the mature light chain variable region comprises the three IMGT light chain CDRs of SEQ ID NOs:78-80; the mature heavy chain variable region comprises the three IMGT heavy chain CDRs of SEQ ID NOs:85-87 and the mature light chain variable region comprises the three IMGT light chain CDRs of SEQ ID NOs:88-90; the mature heavy chain variable region comprises the three IMGT heavy chain CDRs of SEQ ID NOs:95-97 and the mature light chain variable region comprises the three IMGT light chain CDRs of SEQ ID NOs:98-100; the mature heavy chain variable region comprises the three IMGT heavy chain CDRs of SEQ ID NOs:105-107 and the mature light chain variable region comprises the three IMGT light chain CDRs of SEQ ID NOs:108-110; or the mature heavy chain variable region comprises the three IMGT heavy chain CDRs of SEQ ID NOs:115-117 and the mature light chain variable region comprises the three IMGT light chain CDRs of SEQ ID NOs:118-120.

Some antibodies comprise a mature heavy chain variable region having an amino acid sequence at least 90% identical to SEQ ID NO:61, and a mature light chain variable region having an amino acid sequence at least 90% identical to SEQ ID NO:62; comprise a mature heavy chain variable region having an amino acid sequence at least 90% identical to SEQ ID NO:71, and a mature light chain variable region having an amino acid sequence at least 90% identical to SEQ ID NO:72; comprise a mature heavy chain variable region having an amino acid sequence at least 90% identical to SEQ ID NO:81, and a mature light chain variable region having an amino acid sequence at least 90% identical to SEQ ID NO:82; comprise a mature heavy chain variable region having an amino acid sequence at least 90% identical to SEQ ID NO:91, and a mature light chain variable region having an amino acid sequence at least 90% identical to SEQ ID NO:92; comprise a mature heavy chain variable region having an amino acid sequence at least 90% identical to SEQ ID NO:101, and a mature light chain variable region having an amino acid sequence at least 90% identical to SEQ ID NO:102; or comprise a mature heavy chain variable region having an amino acid sequence at least 90% identical to SEQ ID NO:111, and a mature light chain variable region having an amino acid sequence at least 90% identical to SEQ ID NO:112.

In some antibodies, the mature heavy chain variable region has an amino acid sequence of SEQ ID NO:61 and the mature light chain variable region has an amino acid sequence of SEQ ID NO:62; the mature heavy chain variable region has an amino acid sequence of SEQ ID NO:71 and the mature light chain variable region has an amino acid sequence of SEQ ID NO:72; the mature heavy chain variable region has an amino acid sequence of SEQ ID NO:81 and the mature light chain variable region has an amino acid sequence of SEQ ID NO:82; the mature heavy chain variable region has an amino acid sequence of SEQ ID NO:91 and the mature light chain variable region has an amino acid sequence of SEQ ID NO:92; the mature heavy chain variable region has an amino acid sequence of SEQ ID NO:101 and the mature light chain variable region has an amino acid sequence of SEQ ID NO:102; or the mature heavy chain variable region has an amino acid sequence of SEQ ID NO:111 and the mature light chain variable region has an amino acid sequence of SEQ ID NO:112.

In another aspect, the antibody specifically binds CADM1 and comprises three heavy chain CDRs and three light chain CDRs of antibody HW-97, wherein HW-97 is characterized by a mature heavy chain variable region having an amino acid sequence comprising SEQ ID NO:121 and a mature light chain variable region having an amino acid sequence comprising SEQ ID NO:122; or comprises three heavy chain CDRs and three light chain CDRs of antibody HW-101, wherein HW-101 is characterized by a mature heavy chain variable region having an amino acid sequence comprising SEQ ID NO:131 and a mature light chain variable region having an amino acid sequence comprising SEQ ID NO:132.

In some antibodies, the CDRs are of a definition selected from the group consisting of Kabat, Chothia, Kabat/Chothia Composite, AbM, Contact, and IMGT.

In some such antibodies, the mature heavy chain variable region comprises the three IMGT heavy chain CDRs of SEQ ID NOs:125-127 and the mature light chain variable region comprises the three IMGT light chain CDRs of SEQ ID NOs:128-130; or the mature heavy chain variable region comprises the three IMGT heavy chain CDRs of SEQ ID NOs:135-137 and the mature light chain variable region comprises the three IMGT light chain CDRs of SEQ ID NOs:138-140.

Some antibodies comprise a mature heavy chain variable region having an amino acid sequence at least 90% identical to SEQ ID NO:121, and a mature light chain variable region having an amino acid sequence at least 90% identical to SEQ ID NO:122; or comprise a mature heavy chain variable region having an amino acid sequence at least 90% identical to SEQ ID NO:131, and a mature light chain variable region having an amino acid sequence at least 90% identical to SEQ ID NO:132.

In some antibodies, the mature heavy chain variable region has an amino acid sequence of SEQ ID NO:121 and the mature light chain variable region has an amino acid sequence of SEQ ID NO:122; or the mature heavy chain variable region has an amino acid sequence of SEQ ID NO:131 and the mature light chain variable region has an amino acid sequence of SEQ ID NO:132.

In another aspect, the antibody specifically binds ICAM1 and comprises three heavy chain CDRs and three light chain CDRs of antibody HW-113, wherein HW-113 is characterized by a mature heavy chain variable region having an amino acid sequence comprising SEQ ID NO:141 and a mature light chain variable region having an amino acid sequence comprising SEQ ID NO:142.

In some antibodies, the CDRs are of a definition selected from the group consisting of Kabat, Chothia, Kabat/Chothia Composite, AbM, Contact, and IMGT.

In some such antibodies, the mature heavy chain variable region comprises the three IMGT heavy chain CDRs of SEQ ID NOs:145-147 and the mature light chain variable region comprises the three IMGT light chain CDRs of SEQ ID NOs:148-150.

Some antibodies comprise a mature heavy chain variable region having an amino acid sequence at least 90% identical to SEQ ID NO:141, and a mature light chain variable region having an amino acid sequence at least 90% identical to SEQ ID NO:142. In some antibodies, the mature heavy chain variable region has an amino acid sequence of SEQ ID NO:141: and the mature light chain variable region has an amino acid sequence of SEQ ID NO:142.

Some antibodies are chimeric or humanized.

Some antibodies are IgA1, IgA2, IgD, IgE, IgG1, IgG2, IgG3, IgG4, synthetic IgG, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, a non-depleting IgG, a diabody, or a bivalent antibody.

Some antibodies have the mature light chain variable region fused to a light chain constant region and the mature heavy chain variable region fused to a heavy chain constant region. The heavy chain constant region of some antibodies is a mutant form of a natural human heavy chain constant region which has reduced binding to a Fcγ receptor relative to the natural human heavy chain constant region.

Some antibodies comprise a heavy chain of SEQ ID NO:13 and a light chain of SEQ ID NO:14; comprise a heavy chain of SEQ ID NO:23 and a light chain of SEQ ID NO:24; comprise a heavy chain of SEQ ID NO:33 and a light chain of SEQ ID NO:34; comprise a heavy chain of SEQ ID NO:43 and a light chain of SEQ ID NO:44; comprise a heavy chain of SEQ ID NO:53 and a light chain of SEQ ID NO:54; comprise a heavy chain of SEQ ID NO:63 and a light chain of SEQ ID NO:64; comprise a heavy chain of SEQ ID NO:73 and a light chain of SEQ ID NO:74; comprise a heavy chain of SEQ ID NO:83 and a light chain of SEQ ID NO:84; comprise a heavy chain of SEQ ID NO:93 and a light chain of SEQ ID NO:94; comprise a heavy chain of SEQ ID NO:103 and a light chain of SEQ ID NO:104; comprise a heavy chain of SEQ ID NO:113 and a light chain of SEQ ID NO:114; comprise a heavy chain of SEQ ID NO:123 and a light chain of SEQ ID NO:124; comprise a heavy chain of SEQ ID NO:133 and a light chain of SEQ ID NO:134; or comprise a heavy chain of SEQ ID NO:143 and a light chain of SEQ ID NO:144.

The antibody can be an antibody fragment. The antibody can be a bispecific antibody. The bispecific antibody can be a T-cell engaging bispecific antibody or an NK-cell engaging bispecific antibody. The antibody can be an antibody-based binding protein.

Some antibodies are conjugated to a synthetic molecule. In some embodiments, the synthetic molecule is a cytotoxic agent, a label, a therapeutic radioisotope, or a liposome. In some embodiments, the cytotoxic agent is a small molecule weight toxin, a peptide toxin, or a protein toxin.

In another aspect, any of the antibodies disclosed herein is linked to at least one cytotoxic agent as an antibody drug conjugate. In some embodiments, the cytotoxic agent is a small molecular weight toxin, a peptide toxin, or a protein toxin. In some embodiments, the antibody is chimeric or humanized.

In another aspect, the invention provides a chimeric antigen receptor (CAR), comprising any of the antibodies disclosed herein fused to a transmembrane region and an intracellular T-cell receptor (TCR) signaling domain. In some chimeric antigen receptors, the antibody is chimeric or humanized.

In another aspect, the invention provides a pharmaceutical composition comprising a therapeutically effective amount of any of the antibodies disclosed herein and a pharmaceutically acceptable carrier.

In another aspect, the invention provides a kit comprising any of the antibodies disclosed herein.

In another aspect, the invention provides a nucleic acid encoding the heavy chain and/or light chain of any of the antibodies disclosed herein and a recombinant expression vector comprising the nucleic acid.

In yet another aspect, the invention provides a method of killing or inhibiting the growth of a cell expressing a multiple myeloma cell surface antigen in a subject, comprising administering a pharmaceutical composition disclosed herein to a subject in need thereof, thereby killing or inhibiting the growth of the cell expressing the multiple myeloma cell surface antigen in the subject. In some methods, the cell is a tumor cell.

In some methods, the pharmaceutical composition comprises an antibody drug conjugate (ADC) comprising any of the antibodies disclosed herein and a cytotoxic agent. In some methods, the cytotoxic agent is a small molecular weight toxin, a peptide toxin, or a protein toxin.

In yet another aspect, the invention provides a method of treating a hematologic malignancy in a subject, comprising administering a pharmaceutical composition disclosed herein to the subject having the hematologic malignancy, thereby treating the hematologic malignancy in the subject. In some methods, the hematologic malignancy is multiple myeloma or acute myeloid leukemia.

In some methods, the pharmaceutical composition comprises an antibody drug conjugate (ADC) comprising any of the antibodies disclosed herein and a cytotoxic agent. In some methods, the cytotoxic agent is a small molecular weight toxin, a peptide toxin, or a protein toxin.

In some methods, the antibody is fused to a transmembrane region and an intracellular T-cell receptor (TCR) signaling domain to form a chimeric antigen receptor (CAR).

A further understanding of the nature and advantages of the present invention may be realized by reference to the remaining portions of the specification and claims.

### DESCRIPTION OF THE DRAWINGS

FIGS. 1A, 1B, and 1C depict a Schematic of FBC-seq method. (1A) A Fab-phage library is selected against target (T and non-target (NT) cells in parallel, using Fab-phage biotinylation and capture (FBC) panning. (1B) After phage harvesting, NGS is conducted on the V_{H}-encoding DNA of the selected Fab-phage pool. Amplicons are generated via a nested PCR approach, which incorporates the P5 and P7 adapter sequences. After Illumina MiSeq sequencing, HCDR3 amino acid (aa) sequences are extracted using regular expression patterns identifying rabbit HCDR3 junctional sites. HCDR3 counts are then aggregated for each phage panning output, from which a count matrix is generated. P5 primer binding site (open circle pattern); P7 primer binding site (left-leaning angled line pattern); P5 cluster binding site (no fill), P7 cluster binding site (dotted pattern); sequencing library specific index (right-leaning angled line pattern); HCDR1, HCDR2, and HCDR3 (speckled pattern). TYFC is SEQ ID NO: 171. (1C) The count matrix is then subjected to DESeq2 analysis to find differentially abundant HCDR3 identifiers between the target and non-target Fab-phage repertoires. Volcano plots distinguish mAbs that preferentially bind target cells (right-leaning angled line patterned region) or non-target cells (dotted patterned region) on the basis of statistical significance (y-axis) and differential abundance (x-axis).

FIG. 2A, 2B, 2C, 2D, 2E, 2F depict FBC-seq applied to a pilot experiment with a defined target (ROR1). (2A) Schematic depicting the panning strategy. In the first round of panning (R1), a naïve chimeric rabbit/human Fab library was selected against ROR1⁻ HEK293 cells, via conventional (conv) panning. After reamplification of the R1 output, three anti-ROR1 Fab-phage preparations (409, Top43, and Top54) were diluted into the phage pool by a factor of 1×10⁻⁶. This phage pool is then subjected to a second round of panning (R2) against ROR1− or ROR1++ HEK293 cells in triplicate, with and without the FBC approach (12 replicates in total). The flow cytometry image shows the validation of HEK293-ROR1-TetOn cells stained with the anti-ROR1 Fab 324 and an anti-human Fab secondary antibody in the presence or absence of 1 μg/mL doxycycline (dox). Following NGS, DESeq-2 based differential abundance analysis (as outlined in FIGS. 1A-C) was then implemented to prioritize HCDR3 identifiers enriched in the ROR1++ Fab-phage outputs relative to the ROR1− outputs. (2B) FBC and conventional phage output titers for the ROR1− and ROR1++ triplicates. (2C) Polyclonal Fabs from R1, R2 FBC, and R2 conventional panning outputs were examined for reactivity towards ROR1− HEK293 cells by flow cytometry. For R2 conventional and FBC Fabs, representative histograms for both ROR1− and ROR1++ panning outputs are displayed. These polyclonal R2 outputs mostly contain clones that do not bind to ROR1, as expected. (2D) Relative abundance of reporter anti-ROR1 Fab-phage clones 409, Top43, and Top54 from FBC and conventional panning against ROR1++ cells. Relative abundance was determined from the mean normalized count of the three panning replicates, as informed by NGS. Volcano plots depicting HCDR3 differential abundance in ROR1− vs. ROR1++ differential selection via conventional panning (2E) and FBC panning (2F). Positive Log 2(Differential Abundance) values indicate enrichment in the ROR1++ output relative to the ROR1− output. Library identifiers are indicated with solid-filled circles. HCDR3 identifiers corresponding to ROR1 reporters are indicated with open circles. The HCDR3 identifier of endogenous anti-ROR1 Fab-phage clone 324 is indicated with an open square.

FIG. 3A, 3B, 3C, 3D depict FBC-seq applied to multiple myeloma cell lines. (3A) Schematic depicting the workflow for the FBC-seq method. In the first round of panning, the naïve chimeric rabbit/human Fab-phage library was selected against MM cell lines H929 and U266 separately without the FBC approach (not shown). These round 1 outputs were mixed and then selected in a second round against H929 and U266 cells (target cells), as well as healthy human PBMCs (non-target cells). Phage outputs were then subjected to NGS analysis followed by differential abundance analysis outlined in FIG. 1A-C. After HCDR3s enriched in H929 or U266 outputs were identified, the corresponding phagemids of prioritized HCDR3 identifiers were rescued using an around-the-horn PCR method and produced as soluble Fab fragments. These mAbs were then evaluated for reactivity to MM cell lines, human T cells, as well as HUVECs in preliminary expression profiling assays. (3B) Binding of pAbs from FBC-selected Fab-phage repertoires to H929, U266, HUVECs, and T cells. For assessment of T cell binding, pAbs were incubated with the PBMCs used as non-target cells for panning, and T cells were identified as CD3+. Volcano plots of HCDR3 identifiers selected against MM cell lines H929 (3C) and U266 (3D). HCDR3 identifiers with positive differential abundance values are enriched in the target cell (H929 or U266) panning outputs compared to the control selection condition (PBMCs). Vertical and horizontal dashed black lines represent cutoffs used for subsetting of HCDR3 identifiers for mAb recovery (differential abundance >8 and p value <1×10⁻⁴), in addition to a minimum sample abundance cutoff of 1×10⁻⁴. Datapoints marked with open circle indicate HCDR3 identifiers that were selected for mAb recovery.

FIG. 4A, 4B, 4C depict recovery of phagemids with specific HCDR3 identifiers. (4A) Around-the-horn PCR was conducted with phosphorylated primers (dotted line arrows with P in dotted-line circle), designed to hybridize to conserved J segment sequences, and HCDR3 identifier-specific reverse primers (solid line arrows), designed to hybridize to HCDR3 regions. This PCR produces linear amplicons which require self-ligation to obtain the circularized phagemid from a phagemid library. Transformation of phagemids then enables screening of clones for production of soluble Fabs with the desired specificity. (4B) Agarose gel (1%) of representative around-the-horn amplification for 12 unique HCDR3 identifiers. (4C) NGS relative abundance of HCDR3 identifiers selected for mAb retrieval in H929- or U266-panned phagemid repertoires. Hits are HCDR3 identifiers for which U266- or H929-binding mAbs were obtained with the proper HCDR3 sequence; non-hits are all HCDR3 identifiers which did not. The dashed line indicates the relative abundance cutoff of 10⁻⁴ used in the NGS prioritization filter. The mean of each group is depicted with a horizontal line.

FIG. 5A, 5B depict results of anti-myeloma IgG specificity and competition studies. (5A) Expression profiling of anti-MM IgGs. Flow cytometry-based heatmap indicating the fraction of each cell type stained by each mAb. This panel includes MM cell lines (H929, U266, MM.1R, RPMI-8226) (83), B-cell lines (MEC1, JeKo-1), other hematopoietic lineage cell lines (Jurkat, K562), a non-hematopoietic lineage cell line (HEK293P; did not include HW-8 and HW-81 as indicated by white squares), and healthy donor PBMCs stained with the panel of 15 mAbs. The mAbs are grouped into 4 clusters based on staining profiles. (5B) Epitope binning of anti-MM IgGs. Representative histograms (y-axis, count; x-axis, fluorescence intensity) depicting competition among mAbs with similar cell specificity profiles. For this, H929 cells were first blocked with unlabeled IgG1, then stained with Alexa Fluor 647-labeled IgG1.

FIG. 6A, 6A Continued, and 6B depict results from Multi-omic target identification studies. (6A and 6A Continued) Schematic of target deconvolution workflow. For each of the 4 unique antibody clusters, a representative IgG (HW-1 shown as example) was used to immunoprecipitate the cognate target from MM cell lysates. IP eluates were digested with trypsin and analyzed by LC-MS/MS. Data are presented as a heatmap of spectral counts for the detected genes ≥20; heatmap depicts spectral counts of independent duplicates for each antibody. (6A, left). Stable MM sublines with differential IgG staining were generated by FACS (6A, top right). Sublines were then subjected to transcriptomic profiling and differential abundance analysis. Volcano plots indicating the fold change (x-axis) and statistical significance (y-axis) of each mRNA are shown (6A, bottom right). Transcripts 2-fold enriched in the positive vs. negative populations with a p-adj <0.01 are marked with an open circle. Correlation of IP/MS and FACS/RNA-seq data sets for mAb HW-1 (6A Continued). (6B) Corresponding correlation of IP/MS and FACS/RNA-seq data sets for antibodies HW-45 (top), HW-97 (middle), and HW-113 (bottom).

FIG. 7A, 7A Continued, 7B, 7C, 7D depict IP/MS and FACS/RNA-seq data sets for HW-45, HW-101, and HW-113. (7A) Fc-silenced IgG1 from each mAb cluster were used to IP their targets from H929 cell lysates onto Protein G resin. The IP eluate was analyzed by reducing SDS-PAGE stained with SYPRO Ruby dye (7A) and then digested with trypsin and analyzed by LC-MS/MS. Data are presented as a heatmap of spectral counts ≥20; heatmap depicts spectral counts of independent duplicates for each antibody. (7A continued). White depicts proteins with spectral counts <20. (Note, IP/MS data for HW-1 and TH9-022 (negative control) are also shown in FIG. 6A, left). Cell sublines from MM cell lines RPMI-8226 (7B), H929 (7C), and U266 (7D) with differential expression of the targets (shown as mean fluorescence intensity values) were generated by FACS and stable target expression level following sorting is shown in the histograms (top). RPMI-8226 cells were sorted with HW-97. H929 cells were sorted twice sequentially to establish HW-113 negative and positive cell sublines. U266 cells were sorted twice with HW-45, resulting in a stable target negative cell subline only. Cellular RNA was extracted from each cell subline, subjected to transcriptomics profiling by RNA-seq, and analyzed using DESeq2 (bottom). Volcano plots indicating the fold change (x-axis) and statistical significance (y-axis) of each mRNA are shown. Transcripts marked with an open circle are 2-fold enriched in the positive vs. negative populations and have p-adj <0.01. The gene name is displayed for top notable hits.

FIG. 8A, 8B depict results from target validation studies: (8A) Antibody reactivity was validated by ELISA of selected transcriptomics and proteomics hits. Recombinant purified ECDs (50-150 ng) of each putative target protein were coated, incubated with 150 ng of the indicated primary antibody, and binding was detected using a peroxidase-conjugated anti-human Fab secondary antibody. Heatmap represents averages of technical triplicates. (8B) Target validation via siRNA knockdown. H929 cells were transfected with siRNA pools targeting the putative antibody targets ICAM1, CADM1, and PTPRG. Cells were then subjected to cognate IgG binding analysis via flow cytometry. (NT, non-targeting siRNA).

FIG. 9 depicts results from target validation by siRNA knockdown studies. Shown are heatmaps depicting flow cytometry analyses of siRNA knockdowns. The percent staining (9, top) and MFI (9, bottom) of H929 cells transfected with target-specific commercial siRNA pools against the indicated targets (rows) stained with the indicated Alexa Fluor 647-labeled and Fc-silenced IgG1 4 days after transfection (columns). Note that knockdown of cognate mAb targets did not affect the staining with commercial mAbs against common MM cell markers SLAMF7, CD38, and CD138. NT=non-targeting

FIGS. 10 A-D depict amino acid sequences of mature heavy chain variable regions (FIGS. 10A and 10B) and mature light chain variable regions (FIGS. 10C and 10D) of selected clones of the invention. The amino acid sequences for each antibody clone heavy and light chains, separated into fragments for framework regions (FR) and complementary determining regions (CDR). IMGT/V-QUEST (imgt.org) was used to identify each region; as such, the CDRs are all according to the IMGT definition. The prospective target for each clone is given, with GARS in brackets because it is not known as a cell membrane protein. Light chains are designated kappa (κ) or lambda (λ). FIG. 10A: Clone name (1^{st} column), target validated by ELISA studies (2^{nd} column), SEQ ID NO of mature heavy chain variable region (3^{rd} column), FR1-IMGT (4^{th} column), HCDR1-IMGT (5^{th} column), FR2-IMGT (6^{th} column), and HCDR2-IMGT (7^{th} column). FIG. 10B: Clone name (1^{st} column), target validated by ELISA studies (2^{nd} column), SEQ ID NO of mature heavy chain variable region (3^{rd} column), FR3-IMGT (4^{th} column), HCDR3-IMGT (5^{th} column), and FR4-IMGT (6^{th} column). FIG. 10C: Clone name (1^{st} column), target validated by ELISA studies (2^{nd} column), SEQ ID NO of mature light chain variable region (3^{rd} column), FR1-IMGT (4^{th} column), LCDR1-IMGT (5^{th} column), FR2-IMGT (6^{th} column), and LCDR2-IMGT (7^{th} column). FIG. 10D: Clone name (1^{st} column), target validated by ELISA studies (2^{nd} column), SEQ ID NO of mature light chain variable region (3^{rd} column), FR3-IMGT (4^{th} column), LCDR3-IMGT (5^{th} column), and FR4-IMGT (6^{th} column). Regions of sequences identical to those of another antibody in the figure are underlined.

FIGS. 11 and 11 Continued depict results from SPR analysis of anti-PTPRG Fabs. PTPRG-Fc was immobilized to a CM5 Biacore chip via the Fc domain, a titration of the indicated soluble Fabs was injected (lines marked 100 nM; 50 nM; 25 nM; 12.5 nM; 6.25 nM), and SPR sensorgrams were used to determine association rate constant (kₒₙ), dissociation rate constant (k_{off}), and equilibrium dissociation constant (K_{d}=k_{off}/kₒₙ) for HW-1, HW-17, HW-25, HW-42, and HW-70 as mean of triplicates (+/−standard deviation).

FIG. 12 depicts data from FIG. 5A heatmap in tabular form. Expression profiling of anti-MM IgGs. Flow cytometry data indicating the fraction of each cell type stained by each mAb. This panel includes MM cell lines (H929, U266, MM.1R, RPMI-8226) (83), B-cell lines (MEC1, JeKo-1), other hematopoietic lineage cell lines (Jurkat, K562), a non-hematopoietic lineage cell line (HEK293P; did not include HW-8 and HW-81 as indicated by empty cells), and healthy donor PBMCs stained with the panel of 15 mAbs. Cell type (column 1), antibody-bound positive cells (%) for each of antibodies TH9-0022 (column 2); HW-42 (column 3 0; HW-70 (column 4); HW-25 (column 5); HW-1 (column 6); HW-17 (column 7); HW-8 (column 8); HW-16 (column 9); HW-45 (column 10); HW-28 (column 11); HW-56 (column 12); HW-81 (column 13); HW-97 (column 14); HW-101 (column 15); HW-113 (column 16).

FIGS. 13A-K depict data from LC-MS/MS experiments, including data from FIG. 6A, left, heatmap, in tabular form. Spectral counts of independent duplicates for each antibody are shown. Target Gene ID (column 1). Spectral counts for antibody as specified: TH9-0022⁻ (columns 2-3); HW-1 (columns 4-5).

FIGS. 14A-J depict data from LC-MS/MS experiments, including data from FIG. 7A Continued heatmap, in tabular form. Spectral counts of independent duplicates for each antibody are shown. Target Gene ID (column 1). Spectral counts for antibody as specified: TH9-0022 (columns 2-3), HW-1 (columns 4-5), HW-45 (columns 6-7), HW-101 (columns 8-9), HW-113 (columns 10-11).

FIG. 15 depicts data from FIG. 8A heatmap in tabular form. Gene (column 1), ELISA Absorbance (405 nm-570 nm) for antibody as specified: None (column 2); TH9-0022 (column 3); HW-42 (column 4); HW-70 (column 5); HW-25 (column 6); HW-1 (column 7); HW-17 (column 8); HW-8 (column 9) HW-16 (column 10); HW-45 (column 11); HW-28 (column 12); HW-56 (column 13); HW-81 (column 14); HW-97 (column 15); HW-101 (column 16); HW-113 (column 17).

FIG. 16A depicts data from FIG. 9 heatmap (top panel) in tabular form. Transfection (column 1), Cells stained (%) for antibody as specified. None (column 2); TH9-0022 (column 3); HW-42 (column 4); HW-70 (column 5); HW-25 (column 6); HW-1 (column 7); HW-17 (column 8); HW-8 (column 9); HW-16 (column 10); HW-45 (column 11); HW-28 (column 12); HW-56 (column 13); HW-81 (column 14); HW-97 (column 15); HW-101 (column 16); HW-113 (column 17); SLAMF7/CS1 (column 18); CD38 (column 19); CD138 (column 20).

FIG. 16B depicts data from FIG. 9 heatmap (bottom panel) in tabular form. Transfection (column 1), mAb binding (MFI) for antibody as specified. None (column 2); TH9-0022 (column 3); HW-42 (column 4); HW-70 (column 5); HW-25 (column 6); HW-1 (column 7); HW-17 (column 8); HW-8 (column 9); HW-16 (column 10); HW-45 (column 11); HW-28 (column 12); HW-56 (column 13); HW-81 (column 14); HW-97 (column 15); HW-101 (column 16); HW-113 (column 17); SLAMF7/CS1 (column 18); CD38 (column 19); CD138 (column 20).

### BRIEF DESCRIPTION OF THE SEQUENCES

SEQ ID NO:1 sets forth the amino acid sequence of Human PTPRG UniProt No. P23470

SEQ ID NO:2 sets forth the amino acid sequence of Human ICAM-1 UniProt No. P05362

SEQ ID NO:3 sets forth the amino acid sequence of human CADM-1 UniProt No. Q93Y67

SEQ ID NO:4 sets forth the amino acid sequence of Human GARS1 UniProt-Prot No. P41250

SEQ ID NO:5 sets forth the amino acid sequence of mature heavy chain variable region of clone TH9-022

SEQ ID NO:6 sets forth the amino acid sequence of mature light chain variable region of clone TH9-022

SEQ ID NO:7 sets forth the amino acid sequence of heavy chain of clone TH9-022 chimeric rabbit-human IgG1 LA/LA/PG

SEQ ID NO:8 sets forth the amino acid sequence of light chain of clone TH9-022 chimeric rabbit-human IgG1 LA/LA/PG

SEQ ID NO:9 sets forth the Clone TH9-0022 IMGT HCDR3 amino acid sequence

SEQ ID NO:10 sets forth the Clone TH9-0022 IMGT LCDR3 amino acid sequence

SEQ ID NO:11 sets forth the amino acid sequence of mature heavy chain variable region of clone HW-42

SEQ ID NO:12 sets forth the amino acid sequence of mature light chain variable region of clone HW-42

SEQ ID NO:13 sets forth the amino acid sequence of heavy chain of clone HW-42 chimeric rabbit-human IgG1 LA/LA/PG

SEQ ID NO:14 sets forth the amino acid sequence of light chain of clone HW-42 chimeric rabbit-human IgG1 LA/LA/PG

SEQ ID NO:15 sets forth the HW-42 IMGT HCDR1 amino acid sequence

SEQ ID NO:16 sets forth the HW-42 IMGT HCDR2 amino acid sequence

SEQ ID NO:17 sets forth the HW-42 IMGT HCDR3 amino acid sequence

SEQ ID NO:18 sets forth the HW-42 IMGT LCDR1 amino acid sequence

SEQ ID NO:19 sets forth the HW-42 IMGT LCDR2 amino acid sequence

SEQ ID NO:20 sets forth the HW-42 IMGT LCDR3 amino acid sequence

SEQ ID NO:21 sets forth the amino acid sequence of mature heavy chain variable region of clone HW-70

SEQ ID NO:22 sets forth the amino acid sequence of mature light chain variable region of clone NW-70

SEQ ID NO:23 sets forth the amino acid sequence of heavy chain of clone HW-70 chimeric rabbit-human IgG1 LA/LA/PG

SEQ ID NO:24 sets forth the amino acid sequence of light chain of clone HW-70 chimeric rabbit-human IgG1 LA/LA/PG

SEQ ID NO:25 sets forth the HW-70 IMGT HCDR1 amino acid sequence

SEQ ID NO:26 sets forth the HW-70 IMGT HCDR2 amino acid sequence

SEQ ID NO:27 sets forth the HW-70 IMGT HCDR3 amino acid sequence

SEQ ID NO:28 sets forth the HW-70 IMGT LCDR1 amino acid sequence

SEQ ID NO:29 sets forth the HW-70 IMGT LCDR2 amino acid sequence

SEQ ID NO:30 sets forth the HW-70 IMGT LCDR3 amino acid sequence

SEQ ID NO:31 sets forth the amino acid sequence of mature heavy chain variable region of clone HW-25

SEQ ID NO:32 sets forth the amino acid sequence of mature light chain variable region of clone HW-25

SEQ ID NO:33 sets forth the amino acid sequence of heavy chain of clone HW-25 chimeric rabbit-human IgG1 LA/LA/PG

SEQ ID NO:34 sets forth the amino acid sequence of light chain of clone HW-25 chimeric rabbit-human IgG1 LA/LA/PG

SEQ ID NO:35 sets forth the IW-25 IMGT HCDR1 amino acid sequence

SEQ ID NO:36 sets forth the HW-25 IMGT HCDR2 amino acid sequence

SEQ ID NO:37 sets forth the HW-25 IMGT HCDR3 amino acid sequence

SEQ ID NO:38 sets forth the HW-25 IMGT LCDR1 amino acid sequence

SEQ ID NO:39 sets forth the HW-25 IMGT LCDR2 amino acid sequence

SEQ ID NO:40 sets forth the HW-25 IMGT LCDR3 amino acid sequence

SEQ ID NO:41 sets forth the amino acid sequence of mature heavy chain variable region of clone HW-1

SEQ ID NO:42 sets forth the amino acid sequence of mature light chain variable region of clone HW-1

SEQ ID NO:43 sets forth the amino acid sequence of heavy chain of clone HW-1 chimeric rabbit-human IgG1 LA/LA/PG

SEQ ID NO:44 sets forth the amino acid sequence of light chain of clone HW-1 chimeric rabbit-human IgG1 LA/LA/PG

SEQ ID NO:45 sets forth the HW-1 IMGT HCDR1 amino acid sequence

SEQ ID NO:46 sets forth the HW-1 IMGT HCDR2 amino acid sequence

SEQ ID NO:47 sets forth the HW-1 IMGT HCDR3 amino acid sequence

SEQ ID NO:48 sets forth the HW-1 IMGT LCDR1 amino acid sequence

SEQ ID NO:49 sets forth the HW-1 IMGT LCDR2 amino acid sequence

SEQ ID NO:50 sets forth the HW-1 IMGT LCDR3 amino acid sequence

SEQ ID NO:51 sets forth the amino acid sequence of mature heavy chain variable region of clone HW-17

SEQ ID NO:52 sets forth the amino acid sequence of mature light chain variable region of clone HW-17

SEQ ID NO:53 sets forth the amino acid sequence of heavy chain of clone HW-17 chimeric rabbit-human IgG1 LA/LA/PG

SEQ ID NO:54 sets forth the amino acid sequence of light chain of clone HW-17 chimeric rabbit-human IgG1 LA/LA/PG

SEQ ID NO:55 sets forth the HW-17 IMGT HCDR1 amino acid sequence

SEQ ID NO:56 sets forth the HW-17 IMGT HCDR2 amino acid sequence

SEQ ID NO:57 sets forth the HW-17 IMGT HCDR3 amino acid sequence

SEQ ID NO:58 sets forth the HW-17 IMGT LCDR1 amino acid sequence

SEQ ID NO:59 sets forth the HW-17 IMG LCDR2 amino acid sequence

SEQ ID NO:60 sets forth the HW-17 IMGT LCDR3 amino acid sequence

SEQ ID NO:61 sets forth the amino acid sequence of mature heavy chain variable region of clone HW-8

SEQ ID NO:62 sets forth the amino acid sequence of mature light chain variable region of clone HW-8

SEQ ID NO:63 sets forth the amino acid sequence of heavy chain of clone HW-8 chimeric rabbit-human IgG1 LA/LA/PG

SEQ ID NO:64 sets forth the amino acid sequence of light chain of clone HW-8 chimeric rabbit-human IgG1 LA/LA/PG

SEQ ID NO:65 sets forth the HW-8 IMGT HCDR1 amino acid sequence

SEQ ID NO:66 sets forth the HW-8 IMGT HCDR2 amino acid sequence

SEQ ID NO:67 sets forth the HW-8 IMGT HCDR3 amino acid sequence

SEQ ID NO:68 sets forth the HW-8 IMGT LCDR1 amino acid sequence

SEQ ID NO:69 sets forth the HW-8 IMGT LCDR2 amino acid sequence

SEQ ID NO:70 sets forth the HW-8 IMGT LCDR3 amino acid sequence

SEQ ID NO:71 sets forth the amino acid sequence of mature heavy chain variable region of clone HW-16

SEQ ID NO:72 sets forth the amino acid sequence of mature light chain variable region of clone HW-16

SEQ ID NO:73 sets forth the amino acid sequence of heavy chain of clone HW-16 chimeric rabbit-human IgG1 LA/LA/PG

SEQ ID NO:74 sets forth the amino acid sequence of light chain of clone HW-16 chimeric rabbit-human IgG1 LA/LA/PG

SEQ ID NO:75 sets forth the HW-16 IMGT HCDR1 amino acid sequence

SEQ ID NO:76 sets forth the HW-16 IMGT HCDR2 amino acid sequence

SEQ ID NO:77 sets forth the HW-16 IMGT HCDR3 amino acid sequence

SEQ ID NO:78 sets forth the HW-16 IMGT LCDR1 amino acid sequence

SEQ ID NO:79 sets forth the HW-16 IMGT LCDR2 amino acid sequence

SEQ ID NO:80 sets forth the HW-16 IMGT LCDR3 amino acid sequence

SEQ ID NO:81 sets forth the amino acid sequence of mature heavy chain variable region of clone HW-45

SEQ ID NO:82 sets forth the amino acid sequence of mature light chain variable region of clone HW-45

SEQ ID NO:83 sets forth the amino acid sequence of heavy chain of clone HW-45 chimeric rabbit-human IgG1 LA/LA/PG

SEQ ID NO:84 sets forth the amino acid sequence of light chain of clone HW-45 chimeric rabbit-human IgG1 LA/LA/PG

SEQ ID NO:85 sets forth the HW-45 IMGT HCDR1 amino acid sequence

SEQ ID NO:86 sets forth the HW-45 IMGT HCDR2 amino acid sequence

SEQ ID NO:87 sets forth the HW-45 IMGT HCDR3 amino acid sequence

SEQ ID NO:88 sets forth the HW-45 IMGT LCDR1 amino acid sequence

SEQ ID NO:89 sets forth the HW-45 IMGT LCDR2 amino acid sequence

SEQ ID NO:90 sets forth the HW-45 IMGT LCDR3 amino acid sequence

SEQ ID NO:91 sets forth the amino acid sequence of mature heavy chain variable region of clone HW-28

SEQ ID NO:92 sets forth the amino acid sequence of mature light chain variable region of clone HW-28

SEQ ID NO:93 sets forth the amino acid sequence of heavy chain of clone HW-28 chimeric rabbit-human IgG1 LA/LA/PG

SEQ ID NO:94 sets forth the amino acid sequence of light chain of clone HW-28 chimeric rabbit-human IgG1 LA/LA/PG

SEQ ID NO:95 sets forth the HW-28 IMGT HCDR1 amino acid sequence

SEQ ID NO:96 sets forth the HW-28 IMGT HCDR2 amino acid sequence

SEQ ID NO:97 sets forth the HW-28 IMGT HCDR3 amino acid sequence

SEQ ID NO:98 sets forth the HW-28 IMGT LCDR1 amino acid sequence

SEQ ID NO:99 sets forth the HW-28 IMGT LCDR2 amino acid sequence

SEQ ID NO:100 sets forth the HW-28 IMGT LCDR3 amino acid sequence

SEQ ID NO:101 sets forth the amino acid sequence of mature heavy chain variable region of clone HW-56

SEQ ID NO:102 sets forth the amino acid sequence of mature light chain variable region of clone HW-56

SEQ ID NO:103 sets forth the amino acid sequence of heavy chain of clone HW-56 chimeric rabbit-human IgG1 LA/LA/PG

SEQ ID NO:104 sets forth the amino acid sequence of light chain of clone HW-56 chimeric rabbit-human IgG1 LA/LA/PG

SEQ ID NO:105 sets forth the HW-56 IMGT HCDR1 amino acid sequence

SEQ ID NO:106 sets forth the HW-56 IMGT HCDR2 amino acid sequence

SEQ ID NO:107 sets forth the HW-56 IMGT HCDR3 amino acid sequence

SEQ ID NO:108 sets forth the HW-56 IMGT LCDR1 amino acid sequence

SEQ ID NO:109 sets forth the HW-56 IMGT LCDR2 amino acid sequence

SEQ ID NO:110 sets forth the HW-56 IMGT LCDR3 amino acid sequence

SEQ ID NO:111 sets forth the amino acid sequence of mature heavy chain variable region of clone HW-81

SEQ ID NO:112 sets forth the amino acid sequence of mature light chain variable region of clone HW-81

SEQ ID NO:113 sets forth the amino acid sequence of heavy chain of clone HW-81 chimeric rabbit-human IgG1 LA/LA/PG

SEQ ID NO:114 sets forth the amino acid sequence of light chain of clone HW-81 chimeric rabbit-human IgG1 LA/LA/PG

SEQ ID NO:115 sets forth the HW-81 IMGT HCDR1 amino acid sequence

SEQ ID NO:116 sets forth the HW-81 IMGT HCDR2 amino acid sequence

SEQ ID NO:117 sets forth the HW-81 IMGT HCDR3 amino acid sequence

SEQ ID NO:118 sets forth the HW-81 IMGT LCDR1 amino acid sequence

SEQ ID NO:119 sets forth the HW-81 IMGT LCDR2 amino acid sequence

SEQ ID NO:120 sets forth the HW-81 IMGT LCDR3 amino acid sequence

SEQ ID NO:121 sets forth the amino acid sequence of mature heavy chain variable region of clone HW-97

SEQ ID NO:122 sets forth the amino acid sequence of mature light chain variable region of clone HW-97

SEQ ID NO:123 sets forth the amino acid sequence of heavy chain of clone HW-97 chimeric rabbit-human IgG1 LA/LA/PG

SEQ ID NO:124 sets forth the amino acid sequence of light chain of clone HW-97 chimeric rabbit-human IgG1 LA/LA/PG

SEQ ID NO:125 sets forth the HW-97 IMGT HCDR1 amino acid sequence

SEQ ID NO:126 sets forth the HW-97 IMGT HCDR2 amino acid sequence

SEQ ID NO:127 sets forth the HW-97 IMGT HCDR3 amino acid sequence

SEQ ID NO:128 sets forth the HW-97 IMGT LCDR1 amino acid sequence

SEQ ID NO:129 sets forth the HW-97 IMGT LCDR2 amino acid sequence

SEQ ID NO:130 sets forth the HW-97 IMGT LCDR3 amino acid sequence

SEQ ID NO:131 sets forth the amino acid sequence of mature heavy chain variable region of clone HW-101

SEQ ID NO:132 sets forth the amino acid sequence of mature light chain variable region of clone HW-101

SEQ ID NO:133 sets forth the amino acid sequence of heavy chain of HW-101 chimeric rabbit-human IgG1 LA/LA/PG

SEQ ID NO:134 sets forth the amino acid sequence of light chain of clone HW-101 chimeric rabbit-human IgG1 LA/LA/PG

SEQ ID NO:135 sets forth the HW-101 IMGT HCDR1 amino acid sequence

SEQ ID NO:136 sets forth the HW-101 IMGT HCDR2 amino acid sequence

SEQ ID NO:137 sets forth the HW-101 IMGT HCDR3 amino acid sequence

SEQ ID NO:138 sets forth the HW-101 IMGT LCDR1 amino acid sequence

SEQ ID NO:139 sets forth the HW-101 IMGT LCDR2 amino acid sequence

SEQ ID NO:140 sets forth the HW-101 IMGT LCDR3 amino acid sequence

SEQ ID NO:141 sets forth the amino acid sequence of mature heavy chain variable region of clone HW-113

SEQ ID NO:142 sets forth the amino acid sequence of mature light chain variable region of clone HW-113

SEQ ID NO:143 sets forth the amino acid sequence of heavy chain of HW-113 chimeric rabbit-human IgG1 LA/LA/PG

SEQ ID NO:144 sets forth the amino acid sequence of light chain of clone HW-113 chimeric rabbit-human IgG1 LA/LA/PG

SEQ ID NO:145 sets forth the HW-113 IMGT HCDR1 amino acid sequence

SEQ ID NO:146 sets forth the HW-113 IMGT HCDR2 amino acid sequence

SEQ ID NO:147 sets forth the HW-113 IMGT HCDR3 amino acid sequence

SEQ ID NO:148 sets forth the HW-113 IMGT LCDR1 amino acid sequence

SEQ ID NO:149 sets forth the HW-113 IMGT LCDR2 amino acid sequence

SEQ ID NO:150 sets forth the HW-113 IMGT LCDR3 amino acid sequence

SEQ ID NO:151 sets forth the nucleotide sequence of IgHJ 2/4:

SEQ ID NO:152 sets forth the nucleotide sequence of IgHJ 3/5

SEQ ID NO:153 sets forth the nucleotide sequence of IgHJ 6

SEQ ID NO:154 sets forth the nucleotide sequence of IgHJ variant 1/2

SEQ ID NO:155 sets forth the nucleotide sequence of IgHJ variant 3

SEQ ID NO:156 sets forth the nucleotide sequence of P5-ch1:

SEQ ID NO:157 sets forth the nucleotide sequence of P7-PELB:

SEQ ID NO:158 sets forth the nucleotide sequence of P5-universal

SEQ ID NO:159 sets forth the nucleotide sequence of P-Index-1

SEQ ID NO:160 sets forth the nucleotide sequence of P-Index-2:

SEQ ID NO:161 sets forth the nucleotide sequence of P-Index-3

SEQ ID NO:162 sets forth the nucleotide sequence of P-Index-4:

SEQ ID NO:163 sets forth the nucleotide sequence of P-Index-5:

SEQ ID NO:164 sets forth the nucleotide sequence of P-Index-6:

SEQ ID NO:165 sets forth the nucleotide sequence of P-Index-7:

SEQ ID NO:166 sets forth the nucleotide sequence of P-Index-8:

SEQ ID NO:167 sets forth the nucleotide sequence of P-Index-9:

SEQ ID NO:168 sets forth the nucleotide sequence of P-Index-10:

SEQ ID NO:169 sets forth the nucleotide sequence of P-Index-11:

SEQ ID NO:170 sets forth the nucleotide sequence of P-Index-12:

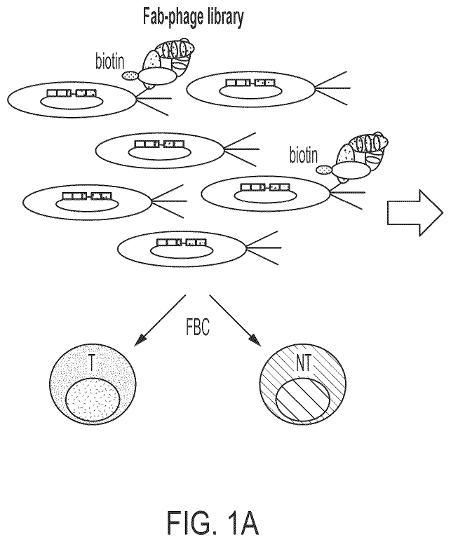

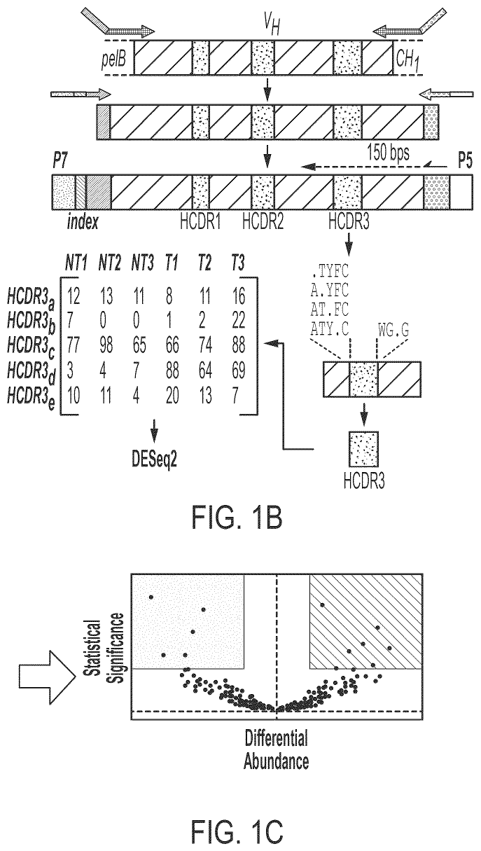

SEQ ID NO:171 sets forth the amino acid sequence in FIG. 1B

SEQ ID NO:172 sets forth the amino acid sequence of an immunoglobulin heavy chain signal peptide.

SEQ ID NO:173 sets forth the amino acid sequence of a linker.

SEQ ID NO:174 sets forth the amino acid sequence of a hinge.

### Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anand (Eds.), Anmol Publications Pvt. Ltd. (2002); and A Dictionary of Biology (Oxford Paperback Reference), Martin and Hine (Eds.). Oxford University Press (4^{th} ed., 2000). In addition, the following definitions are provided to assist the reader in the practice of the invention.

The term “antibody” also synonymously called “immunoglobulins” (Ig), or “antigen-binding fragment” refers to polypeptide chain(s) which exhibit a specific monovalent, bivalent or polyvalent binding to a given antigen, epitope or epitopes. Unless otherwise noted, antibodies or antigen-binding fragments used in the invention can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments, bispecific and other designer antibodies that are described below or well known in the art (see, e.g., Serafini, J Nucl. Med. 34:533-6, 1993), T-cell engaging bispecific antibodies (T-biAbs), NK-cell engaging bispecific antibodies (NK-biAbs), antibodies conjugated to other molecules, such as antibody drug conjugates, radioimmunoconjugates, antibody based binding proteins and chimeric antigen receptors including VH and VL regions.

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region of most IgG isotypes (subclasses) is comprised of three domains, C_{H1}, C_{H2} and C_{H3}, some IgG isotypes, like IgM or IgE comprise a fourth constant region domain, C_{H4}. Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991) or IMGT (imgt.org).

An “antigen binding site” or “antigen-binding region” of an immunoglobulin of the present invention typically contains six complementarity determining regions (CDRs) within each variable domain, and which contribute in varying degrees to the affinity of the binding site for antigen. In each variable domain there are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences and/or structural information from antibody/antigen complexes. Also included within the scope of the invention are functional antigen binding sites comprised of fewer CDRs (i.e., where binding specificity is determined by three, four or five CDRs). Less than a complete set of 6 CDRs can be sufficient for binding to some binding targets. Thus, in some instances, the CDRs of a V_{II} or a V_{L} domain alone will be sufficient. Furthermore, certain antibodies, in particular rabbit antibodies, might have non-CDR-associated binding sites for an antigen. Such binding sites are specifically included within the present definition.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which HCDR1 is a composite of Chothia and Kabat CDRs; the AbM definition used by Oxford Molecular's antibody modelling software; and, the contact definition of Martin et al (bioinfo.org.uk/abs), and IMGT definition (imgt.org/IMGTScientificChart/Numbering/IMGTnumberingCDR_VK.html) (see Table 1). Kabat provides a widely used numbering convention (Kabat numbering) in which corresponding residues between different heavy chains or between different light chains are assigned the same number. When an antibody is said to comprise CDRs by a certain definition of CDRs (e.g., IMGT) that definition specifies the minimum number of CDR residues present in the antibody (i.e., the IMGT CDRs). It does not exclude that other residues falling within another conventional CDR definition but outside the specified definition are also present.

TABLE 1
Conventional Definitions of CDRs Using Kabat Numbering

| | | | Composite | | | |
|---|---|---|---|---|---|---|
| | | | of Chothia | | | |
| | | | & | | | |
| Loop | Kabat | Chothia | Kabat | AbM | Contact | IMGT |
| L1 | L24--L34 | L24--L34 | L24--L34 | L24--L34 | L30--L36 | L27- |
| | | | | | | L32 |
| L2 | L50--L56 | L50--L56 | L50--L56 | L50--L56 | L46--L55 | L50- |
| | | | | | | L52 |
| L3 | L89--L97 | L89--L97 | L89--L97 | L89--L97 | L89--L96 | L89- |
| | | | | | | L97 |
| H1 | H31--H35B | H26-- | H26--H35B* | H26--H35B | H30--H35B | H26- |
| | | H32 . . . H34* | | | | H33 |
| H2 | H50--H65 | H52--H56 | H50--H65 | H50--H58 | H47--H58 | H51- |
| | | | | | | H56 |
| H3 | H95--H102 | H95--H102 | H95--H102 | H95--H102 | H93--H101 | H93- |
| | | | | | | H102 |

*HCDR1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia HCDR1 loop ends at H32. If only H35A is present, it ends at H33. If both H35A and H35B are present, it ends at H34.

An “antibody-based binding protein”, as used herein, may represent any protein that contains at least one antibody-derived V_{H}, V_{L}, in the context of other non-immunoglobulin, or non-antibody derived components. Such antibody-based proteins include, but are not limited to (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments (antigen-binding fragments).

“Binding affinity” is generally expressed in terms of equilibrium association or dissociation constants (K_{A} or K_{D}, respectively), which are in turn reciprocal ratios of dissociation and association rate constants (k_{off} and kₒₙ, respectively). Thus, equivalent affinities may correspond to different rate constants, so long as the ratio of the rate constants remains the same. The binding affinity of an antibody is usually be expressed as the K_{D} of a monovalent fragment (e.g. a F_{ab} fragment) of the antibody, with K_{D} values in the single-digit nanomolar range or below (subnanomolar or picomolar) being considered as very high and of therapeutic and diagnostic relevance.

As used herein, the term “binding specificity” refers to the selective affinity of one molecule for another such as the binding of antibodies to antigens (or an epitope or antigenic determinant thereof), receptors to ligands, and enzymes to substrates. Thus, all monoclonal antibodies that bind to a particular antigenic determinant of an entity (e.g., a specific epitope of a multiple myeloma cell surface antigen, including any of PTPRG, ICAM1, CADM1, or GARS) are deemed to have the same binding specificity for that entity.

The term “Antibody Drug Conjugate”, or “ADC” refers to an antibody to which a therapeutically active substance or an active pharmaceutical ingredient (API) has been covalently linked, such that the therapeutically active substance or an active pharmaceutical ingredient (API) can be targeted to the binding target of the antibody to exhibit its pharmacologic function. Linkage can be direct via a chemical bond or indirect through a linker. Some linkers are cleavable under physiological conditions, whereas other linkers are not. The therapeutically active substance or an active pharmaceutical ingredient can be a cellular toxin that is able to effect killing of the cells targeted by the ADCs, preferably malignant or cancer cells. The covalent attachment of a therapeutically active substance, an active pharmaceutical ingredient or a cellular toxin can be performed in a non-site specific manner using standard chemical linkers that couple payloads to lysine or cysteine residues, or, preferably the conjugation is performed in a site-specific manner, that allows full control of conjugation site and drug to antibody ratio (DAR) of the ADC to be generated.

The term “radioimmunoconjugate”, or “RIC” refers to an antibody to which a radionuclide has been attached, such that the radionuclide can be targeted to the binding target of the antibody to deliver cytotoxic radiation to a target cell, for example a cancer cell. In some embodiments, the radionuclide is directly attached to the antibody via a covalent bond or is attached to the antibody via a chelation complex.

The terms “T-cell engaging bispecific antibody (T-biAb)” and “NK-cell engaging bispecific antibody (NK-biAb)” refer to a bispecific antibody that simultaneously binds to an activating receptor on T cells (e.g., CD3) and NK cells (e.g., CD16), respectively, and a target-associated antigen on a target cell, activates T/NK cells, and redirects T/NK cells' cytotoxicity against the target cell.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or phage), combining agents and cells, or combining two populations of different cells. Contacting can occur in vitro, e.g., mixing an antibody and a cell or mixing a population of antibodies with a population of cells in a test tube or growth medium. Contacting can also occur in a cell or in situ, e.g., contacting two polypeptides in a cell by coexpression in the cell of recombinant polynucleotides encoding the two polypeptides, or in a cell lysate. Contacting can also occur in vivo inside a subject, e.g., by administering an agent to a subject for delivery the agent to a target cell.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Natl. Acad. Sci. USA 85:2444, 1988; by computerized implementations of these algorithms (GAP, BESTFIT, FASTA, and TFASTA in the Wisconsin Genetics Software Package, Genetics Computer Group, Madison, WI); or by manual alignment and visual inspection (see, e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nucleic Acids Res. 25:3389-3402, 1977; and Altschul et al., J. Mol. Biol. 215:403-410, 1990, respectively.

The term “subject” refers to human and non-human animals (especially non-human mammals). The term “subject” is used herein, for example, in connection with therapeutic and diagnostic methods, to refer to human or animal subjects. Animal subjects include, but are not limited to, animal models, such as, mammalian models of conditions or disorders associated a hematologic malignancy such as multiple myeloma or acute myeloid leukemia (AML). Other specific examples of non-human subjects include, e.g., cows, horses, sheep, pigs, cats, dogs, mice, rats, rabbits, guinea pigs, monkeys.

Artificial T cell receptors (also known as chimeric T cell receptors, chimeric immunoreceptors, chimeric antigen receptors (CARs) or T-bodies) are engineered receptors, which graft an arbitrary specificity onto an immune effector cell. Typically, these receptors are used to graft the specificity of a monoclonal antibody onto a T cell; with transfer of their coding sequence facilitated by retroviral or lentiviral vectors or by transposons. CAR-engineered T cells (also abbreviated CAR-T cells) are genetically engineered T cells armed with chimeric receptors whose extracellular recognition unit is comprised of an antibody-derived recognition domain and whose intracellular region is derived from lymphocyte stimulating moiety(ies). The structure of the prototypic CAR is modular, designed to accommodate various functional domains and thereby to enable choice of specificity and controlled activation of T cells. The preferred antibody-derived recognition unit is a single chain variable fragment (scFv) that combines the specificity and binding residues of both the heavy and light chain variable regions of a monoclonal antibody. The most common lymphocyte activation moieties include a T-cell costimulatory (e.g. CD28) domain in tandem with a T-cell triggering (e.g. CD3zeta) moiety. By arming effector lymphocytes (such as T cells and natural killer cells) with such chimeric receptors, the engineered cell is redirected with a predefined specificity to any desired target antigen, in a non-HLA restricted manner. CAR constructs are introduced ex vivo into T cells from peripheral lymphocytes of a given patient using retroviral or lentiviral vectors or transposons. Following infusion of the resulting CAR-engineered T cells back into the patient, they traffic, reach their target site, and upon interaction with their target cell or tissue, they undergo activation and perform their predefined effector function. Therapeutic targets for the CAR approach include cancer and HIV-infected cells, or autoimmune effector cells. CAR-engineered NK cells are conceptually and architecturally related to CAR-engineered T cells.

The terms “treat,” “treating,” “treatment,” and “therapeutically effective” used herein do not necessarily imply 100% or complete treatment. Rather, there are varying degrees of treatment recognized by one of ordinary skill in the art as having a potential benefit or therapeutic effect. In this respect, the inventive method can provide any amount of any level of treatment. Furthermore, the treatment provided by the inventive method can include the treatment of one or more conditions or symptoms of the disease being treated.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

Compositions or methods “comprising” or “including” one or more recited elements may include other elements not specifically recited. When the disclosure refers to a feature comprising specified elements, the disclosure should alternative be understood as referring to the feature consisting essentially of or consisting of the specified elements. Moreover, elements that are shown or described as being combined with other elements, can, in various embodiments, exist as stand-alone elements.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value. Unless otherwise apparent from the context, the term “about” encompasses values within ±5% or ±10% of a stated value.

The singular forms of the articles “a,” “an,” and “the” include plural references unless the context clearly dictates otherwise.

### Abbreviations

aa, amino acid; BSA, bovine serum albumin; CADM1, cell adhesion molecule 1; CAR-T, chimeric antigen receptor T cell; DMEM, Dulbecco's Modified Eagle's Medium; DPBS, Dulbecco's PBS; ECD, extracellular domain; FACS, fluorescence-activated cell sorting; FBC, Fab-phage biotinylation and capture; FBS, fetal bovine serum; FDA, Food and Drug Administration; GARS, glycyl-tRNA synthetase; HCDR3, heavy chain complementarity determining region 3; huCH1, human constant heavy chain domain 1; huCL, human constant light chain domain; ICAM1, intercellular adhesion molecule 1; IMAC, immobilized metal affinity chromatography; IP, immunoprecipitation; LCDR3, light chain complementarity determining region 3; LC-MS/MS, liquid chromatography tandem mass spectrometry; mAb, monoclonal antibody; MM, multiple myeloma; NGS, next-generation sequencing; NT, non-target; PBMCs, peripheral blood mononuclear cells; PBS, phosphate-buffered saline; PS, penicillin-streptomycin; PTM, post-translational modification; PTPRG, protein tyrosine phosphatase receptor type G; rbVH, rabbit variable heavy chain domain; rbVL, rabbit variable light chain domain; RT, room temperature; SCLC, small cell lung cancer; SPR, surface plasmon resonance; TBS, Tris-buffered saline; TCR, T cell receptor; WCP, whole-cell panning

### DETAILED DESCRIPTION

### I. Overview

In one aspect, the invention provides antibodies that specifically bind PTPRG, CADM1, ICAM1, and GARS. Exemplary antibodies were identified from highly diverse phage-display libraries of non-immunized rabbits by differential selection on multiple myeloma cells versus healthy donor peripheral blood mononuclear cells (PBMCs). By this strategy antibodies for PTPRG, CADM1, ICAM1, and GARS of high quality have been identified. Furthermore, the invention provides chimeric full-length antibodies of the rabbit variable domains fused to the constant region domains of human IgG₁ antibodies. The invention also provides humanized or chimeric full-length antibodies of the rabbit CDRs grafted into human variable region frameworks or rabbit variable region frameworks, in either case fused to the constant region domains of human IgG₁ antibodies.

The present invention provides monoclonal antibodies and related antibody-based binding proteins and antibody fragments (antigen-binding fragments) thereof that specifically recognize multiple myeloma cell surface antigens PTPRG, CADM1, ICAM1, and GARS. Exemplary antibodies of the invention are HW-42, HW-70, HW-25, HW-1, HW-17, HW-8, HW-16, HW-45, HW-28, HW-56, HW-81, HW-97, HW-101, and HW-113. Exemplary antibodies of the invention binding PTPRG are HW-42, HW-70, HW-25, HW-1, and HW-17. Exemplary antibodies of the invention binding GARS are HW-8, HW-16, HW-45, HW-28, HW-56, and HW-81. Exemplary antibodies of the invention binding CADM1 are HW-97 and HW-101. An exemplary antibody of the invention binding ICAM1 is HW-113.

The invention also provides antibody drug conjugates (ADCs), radioimmunoconjugates (RICs), T-cell engaging bispecific antibodies (T-biAbs), NK-cell engaging bispecific antibodies (NK-biAbs), chimeric antigen receptors (CARs) derived from the antibodies binding PTPRG, CADM1, ICAM1, and GARS described herein. Further provided in the invention are methods of using these antibody agents and related compositions in therapeutic and diagnostic applications for diseases and conditions with associated abnormal or elevated PTPRG, CADM1, ICAM1, and GARS expression, e.g., hematologic malignancies including multiple myeloma (MM) and acute myeloid leukemia (AML).

### II. Antibody Discovery by Phage Display

Monoclonal antibody (mAb)-based biologics are well established treatments of cancer. Antibody discovery campaigns are typically directed at a single target of interest, which inherently limits the possibility of uncovering novel antibody specificities or functionalities. Here, we present a target-unbiased approach for antibody discovery that relies on generating mAbs against native target cell surfaces via phage display. This method combines a previously reported method for improved whole-cell phage display selections with next-generation sequencing analysis to efficiently identify mAbs with the desired target cell reactivity. Applying this method to multiple myeloma cells yielded a panel of >50 mAbs with unique sequences and diverse reactivities. To uncover the identities of the cognate antigens recognized by this panel, representative mAbs from each unique reactivity cluster were used in a multi-omic target deconvolution approach. From this, four distinct targets were identified: ICAM1, PTPRG, CADM1, and GARS.

Recently, we developed a more efficient WCP approach for the selection of Fab-phage libraries termed Fab-phage biotinylation and capture (FBC) [16]. In the FBC method, phage-displayed Fab are enzymatically biotinylated, which allows for the removal of bald (Fab- and biotin-less) phage post-elution and significantly increases WCP efficiency. However, the implementation of FBC for target-agnostic WCP is limited by the low-throughput process of colony screening, which is typically limited to 10² or 10³ mAb clones, while selected libraries can have 10⁴⁻⁷ unique mAbs. Thus, low-abundance clones of interest are potentially missed. Another complication that arises in WCP is the outgrowth of immunodominant mAbs that bind irrelevant epitopes (i.e. those not specific for the target cell type). A common approach to this problem is negative selection or depletion on non-target cells or vesicles, but this may eliminate mAbs against targets with low basal expression on the non-target cells, even if it is highly over-expressed on the target cells [5]. New developments in next-generation sequencing (NGS) technologies enable a more comprehensive assessment of output antibody diversity, and have the potential to address these limitations of WCP [17-19]. NGS also allows for the removal of sequence redundancy and therefore reduces the degree of back-end screening required. Although others have reported using NGS for analyzing WCP outputs [20-22], none were done using bald phage depletion. Combining these two methods has the potential to significantly enhance antibody discovery workflows by reducing selection noise and increasing the functional coverage of selected antibody libraries.

Another problem common to all target-agnostic drug discovery efforts is the identification of the cognate targets. In the case of antibodies, immunoprecipitation (IP) is the traditional method of choice, yet it does not work universally and can require extensive optimization, prohibiting the parallel deorphanization of multiple candidate mAbs. Native protein arrays, in which individual membrane proteins are overexpressed on the cell surface, provide useful information for on- and off-target interactions but are expensive and limited to one or two cell types. Endogenous expression of membrane proteins can at times impede successful target deconvolution [23, 24].

Addressing these challenges, the overall objective of this work was to develop a streamlined method for antibody drug and target discovery using WCP. Accomplishing a first aim, we arrived at a method, FBC-seq, that hinges on the differential selection of Fab-phage libraries on target versus non-target cell types and determines the enrichment of mAbs captured by the target cell population using NGS. The method was validated in a binary setting by selecting Fab-phage on an isogenic ROR1− and ROR1+ cell pair and computationally identifying anti-ROR1 clones enriched in the target cell selection. To explore the utility for target discovery in MM, the FBC approach was employed to differentially select on MM cells versus healthy donor peripheral blood mononuclear cells (PBMCs). The second aim then became the development of methods to robustly identify diverse antigens of these orphan mAbs, and for this a dual proteomics and comparative transcriptomics approach proved highly effective.

In this work, a target-agnostic phage display method was developed and applied to identify mAbs binding MM cell surface proteins. To overcome the challenge of bald phage background propagation during WCP, the FBC approach was employed to select a naïve chimeric rabbit/human Fab-phage library. Since many mAbs would naturally target receptors common to the hematopoietic lineage, panning was done in parallel on healthy PBMCs and MM cells, followed by NGS analysis of the panning output to computationally identify antibody clones that bind to MM-enriched antigens. Over 150 unique HCDR3s were found, 55 Fabs were confirmed to have MM cell reactivity, and 14 clones of interest were selected for target deconvolution as purified IgG1s. To efficiently and robustly identify diverse targets, a combination of IP/MS-based proteomics and FACS/RNA-seq-based transcriptomics was employed. This multi-omic target deconvolution approach yielded a single candidate for each of 4 mAb clusters that was validated at the protein level. The antigens included 1 clinically investigated MM target, ICAM1, and 3 potentially novel targets for MM therapy PTPRG, CADM1, and GARS. ICAM1 has been clinically investigated as an antibody target for treating MM, providing compelling evidence that the differential panning selection strategy was successful [8, 33]. CADM1, previously unreported in MM, represents a potential novel target for this indication. While it appears to be present at the protein level in numerous healthy tissues (World Wide Web proteinatlas.org/ENSG00000182985-CADM1/tissue) [34], a CADM1 isoform with restricted expression in small cell lung cancer (SCLC) and testis has been identified [35]. PTPRG, which compared to CADM1 reveals a more restricted expression at the protein level in healthy tissues (World Wide Web proteinatlas.org/ENSG00000144724-PTPRG/tissue), was reported to be overexpressed and mutated in MM, and recent reports indicate it is important for MM cell growth and survival [36]. However, as of January 2023, there are no reports of therapeutic mAbs targeting PTPRG in preclinical or clinical studies. The identification of GARS, which is an aminoacyl-tRNA synthetase that does not have a cell membrane gene ontology (GO) annotation but is known to have essential non-translational functions and is secreted by cancer cells [37], highlights the importance of an unbiased approach to antibody drug and target discovery. GARS has been reported to interact with cadherins CDH6 and CDH18 on the cell surface with high affinity (K_{d}=1 to 3 nM) [38], yet these known receptors were not identified as significant hits in either the IP/MS or FACS/RNA-seq analysis. Although cluster C-45 appears to be polyreactive, specificity and affinity maturation could be employed to achieve monoreactivity for GARS or a GARS complex [39-41], yet cell surface-localized GARS would have to be validated in the context of MM. Since siRNA knockdown of the validated cell surface targets did not change expression of other common MM cell surface markers, antibodies against multiple targets could be combined to increase specificity or avidity, such as logic-gated chimeric antigen receptor T cells (CAR-Ts) [42, 43]. In future studies, further investigation of the identified targets including the staining of primary MM cells will be important in determining the therapeutic or diagnostic utility of these mAbs.

The findings of this work can guide further improvements on WCP selections. A notable observation is an apparent bias towards selecting antibodies against targets with large extracellular domains (ECDs). The ECDs of PTPRG, ICAM1, and CADM1 comprise 716, 452, and 329 amino acids (aa), respectively, and the ECDs of the potential GARS receptors, CDH6 and CDH18, comprise 562 and 555 aa, respectively. Both ICAM1 [44] and CADM1 [45] have been identified previously using target-agnostic WCP, and one can speculate that their common role in cell adhesion—they project out from the cell and have evolved to display thermodynamically favored binding sites for other receptors—may explain their recurrent identification using these methods [46]. In another target-agnostic WCP campaign on primary chronic lymphocytic leukemia cells, we identified Siglec-6 as the target of a dominating mAb cluster and mapped its epitope to an N-terminal (membrane-distal) region in its 321 aa ECD [47]. Thus, Fab-phage library selections on whole cells may be biased or restricted to epitopes that extend far from the cell membrane, out from the glycocalyx, 100 Å or more from the cell membrane. Perhaps increasing the incubation time or trimming back the glycocalyx may improve mAb accessibility to more membrane-proximal epitopes [48, 49]. Overall, the selected targets have low expression on PBMCs, based on our staining data and as reported by databases such as the Human Protein Atlas. However, since PBMCs were the only target-negative cell type used, the targets can be found in other tissues and cell types including endothelial cells and fibroblasts. These expression patterns may be manageable, given that one of the targets, ICAM1, appears to be broadly expressed, while the clinically investigated mAbs against it appear to be safe [33]. Nonetheless, future implementations of the FBC-seq methodology could include the use of primary samples, and multiple non-target cell types to increase target cell specificity. Notably, a commonly used negative selection approach in which phage are first incubated with non-target cells before incubating the depleted phage with target cells risks the loss of rare clones and may also eliminate binders against targets with low basal expression on non-target cells, even if they are highly over-expressed on the target cells. In contrast, our approach is conducting parallel positive selections on non-target and target cells and comparing the outcome by NGS. As such, it mitigates the risk of losing mAbs of interest.

In addition to improving the panning method, improvements may also be made to the NGS analysis. A minimalist NGS approach was pursued for FBC-seq, using the HCDR3 as the clonal identifier since this is typically the paratope and the most diverse region [17, 50-53, 81], and accurately reading the entire V_{L} and V_{II} sequences is technically challenging [54, 55, 80]. Another challenge when utilizing NGS for antibody discovery is the retrieval of mAbs from output display repertoires. We elected to use a PCR-based economical solution that obviates the need for immunoglobulin gene synthesis [25, 26, 29, 56, 82]. This strategy proved efficient at HCDR3 clone retrieval, but more than half of recovered clones showed no detectable binding to MM cells. Herein lies the drawback of the HCDR3-mediated recovery approach—the clones recovered could have different sequences upstream in V_{H} or in V_{L}, yet this method is blind to those other sites of variation. Advancements in NGS read length and error rates are expected to eventually facilitate analysis of the entire Fv portion of the antibody at high depth.

Beyond WCP, application of these methods is a generalizable way to improve the depth and efficiency of mAb recovery from display repertoires. One particularly appealing application is in the generation of mAbs against conformationally sensitive integral membrane proteins such as GPCRs or ion channels, which are notoriously challenging to handle in a purified form [57]. While these protein classes represent intriguing targets for antibody-based drugs due to the diverse physiological roles they mediate [58], raising mAbs of high affinity and specificity against them remains a major hurdle [59]. Our method may also find application in the emerging field of T cell receptor (TCR)-mimicking mAbs that recognize subtle differences among peptides presented by major histocompatibility complexes and as such require stringent differential selection and screening workflows [60].

The use of complementary IP/MS and FACS/RNA-seq strategies for target identification proved effective at filtering out noise inherent to each individual method. The IP/MS assay had high background due to proteins that are highly abundant or co-immunoprecipitated, while the FACS/RNA-seq experiment identified all mRNAs differentially expressed in cells that express higher levels of the cell surface antigen, some of which may have a functional relationship with the target. The distinctive sources of noise in each of these experiments are eliminated when correlating the two datasets, as there were no non-antigens that were dually identified in both approaches. Custom membrane protein array technology [23], which identified Siglec-6 in a previous target-agnostic WCP campaign [32], was attempted for several mAb cluster-representing IgG1s, and despite PTPRG, ICAM1, and CADM1 being present in the collection, only CADM1 was identified (data not shown). The failure of this approach may be due to technical limitations including the use of chemical fixation reagents and a non-hematopoietic host cell type (HEK293), either of which could result in epitope loss.

The FBC-seq method presented here allows for the robust identification of multiple potential therapeutic antibodies and cell surface antigens in parallel. A key feature of this approach is the target-agnostic nature of Fab-phage library-driven concerted antibody and antigen discovery which complements target-defined, antigen-driven antibody discovery initiated by transcriptomic and proteomic data. Since the focus of this study was validation of the FBC-seq method, antibody binding and specificity were prioritized. However, a function-first screen of the bioinformatically-identified mAbs could more directly inform their therapeutic utility. For example, conversion of the hits to, e.g., Fc-active IgG1s capable of mediating effector functions, antibody-drug conjugates, T-cell recruiting bispecific antibodies, or CAR-Ts could facilitate simple cytotoxicity screening on target cells. Importantly, FBC-seq results in mAbs that bind selectively to native and accessible epitopes on target cells, enhancing the depth and efficiency of antibody drug and target discovery.

### III. Protein Targets

PTPRG is a member of the Protein Tyrosine Phosphatase (PTP) family. The mature protein comprises 1426 amino acids of which approximately half form its extracellular domain (ECD) followed by a single transmembrane region and two intracellular phosphatase domains. The ECD contains a carbonic anhydrase (CAH)-like domain and a fibronectin type III domain. An exemplary PTPRG amino acid sequence is UniProt P23470-1, provided as SEQ ID NO:1.

Reference to PTPRG includes known natural variations which are listed in the Swiss-Prot database and permutations thereof, as well as mutations associated with pathologies including MM and other cancers. Additionally, reference to PTPRG includes PTPRG with known post-translational modifications. Examples of known post-translational modifications are listed in the UniProtKB/Swiss-Prot database. Unless otherwise apparent from context, reference to PTPRG, or its fragments includes the natural human amino acid sequences including isoforms, mutants, and allelic variants thereof. Unless otherwise apparent from the context, reference to PTPRG means a natural human form of PTPRG including all isoforms, including soluble forms, irrespective of whether posttranslational modification (e.g., phosphorylation, glycation, or acetylation) is present.

Intercellular adhesion molecule (ICAM) proteins are ligands for the leukocyte adhesion protein LFA-1 (integrin alpha-L/beta-2). During leukocyte trans-endothelial migration, ICAM1 (also known as CD54) engagement promotes the assembly of endothelial apical cups through ARHGEF26/SGEF and RHOG activation. ICAM-1 comprises an extracellular domain, a transmembrane domain, and a cytoplasmic domain. An exemplary ICAM1 amino acid sequence is UniProt P05362, provided as SEQ ID NO:2.

Reference to ICAM1 includes known natural variations which are listed in the Swiss-Prot database and permutations thereof, as well as mutations associated with pathologies including MM and other cancers. Additionally, reference to ICAM1 includes ICAM1 with known post-translational modifications. Examples of known post-translational modifications are listed in the UniProtKB/Swiss-Prot database. Unless otherwise apparent from context, reference to ICAM1, or its fragments includes the natural human amino acid sequences including isoforms, mutants, and allelic variants thereof. Unless otherwise apparent from the context, reference to ICAM1 means a natural human form of ICAM1 including all isoforms, including soluble forms, irrespective of whether posttranslational modification (e.g., phosphorylation, glycation, or acetylation) is present.

CADM1 (also known as IGSF4A) mediates homophilic cell-cell adhesion in a Ca²⁺-independent manner and mediates heterophilic cell-cell adhesion with CADM3 and NECTIN3 in a Ca²⁺-independent manner. CADM1 comprises an extracellular domain, a transmembrane domain, and a cytoplasmic domain. CADM1 appears to be present in several healthy tissues The Human Protein Atlas (proteinatlas.org/ENSG00000182985-CADM1/tissue) states “Membranous expression in most glandular cells, high abundance in CNS, testis, islets of Langerhans and basolateral membranes in renal tubules.” An exemplary CADM1 amino acid sequence is UniProt Q9BY67-1, provided as SEQ ID NO:3.

Reference to CADM1 includes known natural variations which are listed in the Swiss-Prot database and permutations thereof, as well as mutations associated with pathologies including MM and other cancers. Additionally, reference to CADM1 includes CADM1 with known post-translational modifications. Examples of known post-translational modifications are listed in the UniProtKB/Swiss-Prot database. Unless otherwise apparent from context, reference to CADM1, or its fragments includes the natural human amino acid sequences including isoforms, mutants, and allelic variants thereof. Unless otherwise apparent from the context, reference to CADM1 means a natural human form of CADM1 including all isoforms, including soluble forms, irrespective of whether posttranslational modification (e.g., phosphorylation, glycation, or acetylation) is present.

GARS (also known as GARS1, Glycine-tRNA ligase, and glycyl-tRNA synthetase): GARS1's canonical function as glycyl-tRNA synthetase is translation in the cytoplasm. However, GARS1 is also known to be secreted by cells including cancer cells and serves noncanonical functions (Guo, M. & Paul Schimmel, P., Nat. Chem. Biol. 9, 145-153, 2013. A recent publication reports that GARS1 interacts with cancer cell surface CDH6 (cadherin 6) via its C-terminal tRNA-binding domain (Park, M. C. et al., Cancer Lett. 539:215698 2022). An exemplary GARS amino acid sequence is UniProt P41250-1, provided as SEQ ID NO:4.

Reference to GARS includes known natural variations which are listed in the Swiss-Prot database and permutations thereof, as well as mutations associated with pathologies including MM and other cancers. Additionally, reference to GARS includes GARS with known post-translational modifications. Examples of known post-translational modifications are listed in the UniProtKB/Swiss-Prot database. Unless otherwise apparent from context, reference to GARS, or its fragments includes the natural human amino acid sequences including isoforms, mutants, and allelic variants thereof. Unless otherwise apparent from the context, reference to GARS means a natural human form of GARS including all isoforms, including soluble forms, irrespective of whether posttranslational modification (e.g., phosphorylation, glycation, or acetylation) is present.

### IV. Antibodies Specifically Binding to a Multiple Myeloma Cell Surface Antigen for Example PTPRG, ICAM1, CADM1, or GARS

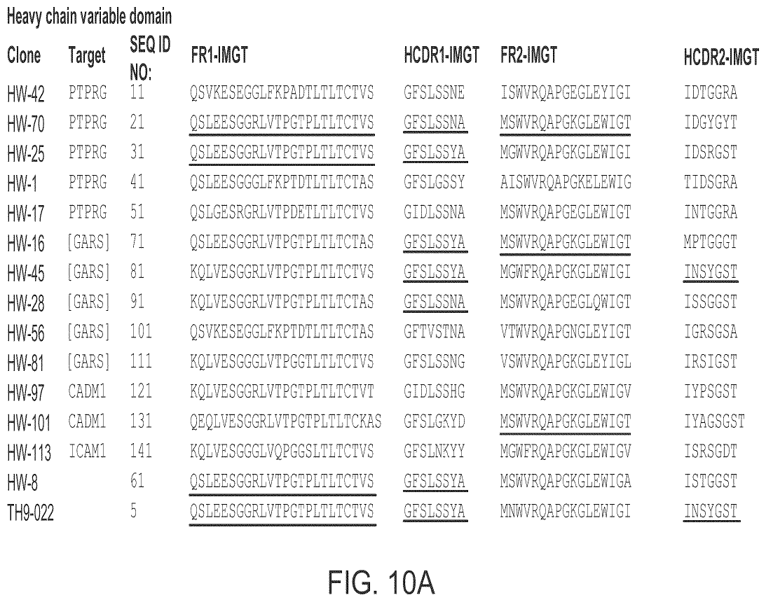

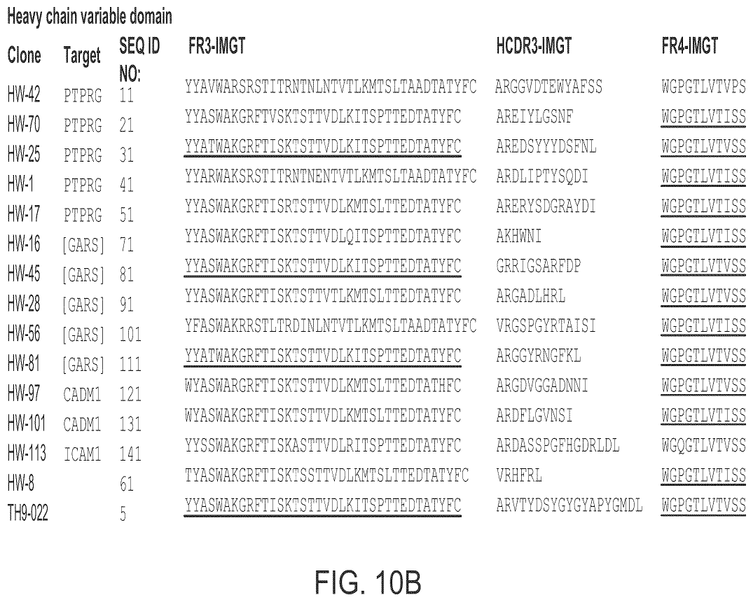

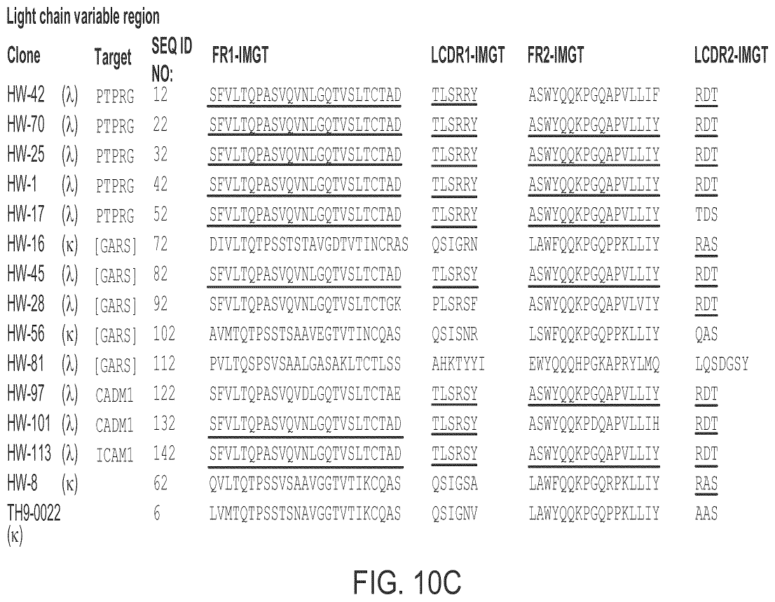

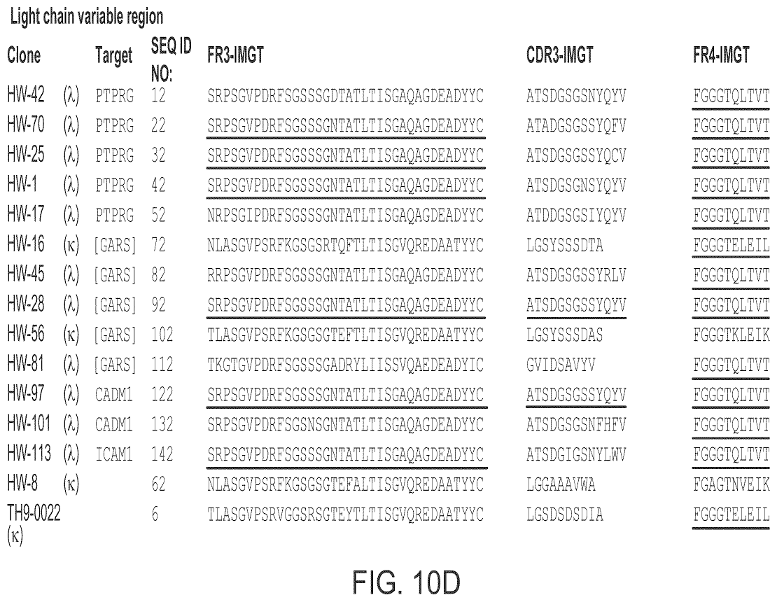

In one aspect, the invention provides antibodies, antibody-based binding proteins, antibody fragments (also termed “antigen-binding fragments”) thereof, ADCs, RICs, T-biAbs, NK-biAbs, or CARs that specifically bind to human PTPRG, ICAM1, CADM1, or GARS with the same binding specificity as that of anti-PTPRG, ICAM1, CADM1, or GARS antibodies exemplified herein (FIG. 10). Antibodies of the invention include intact antibodies (e.g., IgG1 antibodies exemplified herein), antibody fragments (e.g., Fab antibodies exemplified herein), antibody-based binding proteins, antibody fragments thereof, ADCs radioimmunoconjugates (RICs), T- or NK-cell engaging bispecific antibodies (T-biAbs or NK-biAbs), or CARs which contain the antigen-binding portions of an intact antibody that retain capacity to bind the cognate antigen, PTPRG, ICAM1, CADM1, or GARS. Examples of such antibody fragments include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} or V_{L} domain (see, e.g., Ward et al., Nature 341:544-546, 1989); and (vii) an isolated complementarity determining region (CDR) as a linear or cyclic peptide. Examples of antibody-based binding proteins are polypeptides in which the binding domains of the antibodies are combined with other polypeptides or polypeptide domains, e.g. alternative molecular scaffolds, Fc-regions, other functional or binding domains of other polypeptides or antibodies resulting in molecules with addition binding properties, e.g. bi- or multispecific proteins or antibodies. Such polypeptides can create an arrangement of binding or functional domains normally not found in naturally occurring antibodies or antibody fragments.

Antibodies of the invention also encompass antibody fragments, like single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment which are coded for by separate genes, a scFv has the two domains joined (e.g., via recombinant methods) by a synthetic linker. This enables them to be made as a single protein chain in which the V_{L} and V_{H} regions pair to form monovalent molecules.

Antibodies of the present invention also encompass single domain antigen-binding units, which have a camelid scaffold. Animals in the camelid family include camels, llamas, and alpacas. Camelids produce functional antibodies devoid of light chains. The heavy chain variable (V_{H}) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

The various antibodies, antibody-based binding proteins, and antibody fragments thereof described herein can be produced by enzymatic or chemical modification of the intact antibodies, or synthesized de novo using recombinant DNA methodologies, or identified using phage display libraries. Methods for generating these antibodies, antibody-based binding proteins, and antibody fragments thereof are all well known in the art. For example, single chain antibodies can be identified using phage display libraries or ribosome display libraries, gene shuffled libraries (see, e.g., McCafferty et al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plückthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nat. Struct. Biol. 11:500-515, 2002; Ghahroudi et al., FEBS Letters 414:521-526, 1997; and Bond et al., J. Mol. Biol. 332:643-55, 2003. Other types of antigen-binding fragments (e.g., Fab, F(ab′)₂ or Fd fragments) can also be readily produced with routinely practiced immunology methods. See, e.g., Harlow & Lane, Using Antibodies, A Laboratory Manual, Cold Spring Harbor Laboratory Press, Cold Spring Harbor, New York, 1998.

Other antibodies can be obtained by mutagenesis of cDNA encoding the heavy and light chains of an exemplary antibody, such as HW-42, HW-70, HW-25, HW-1, HW-17, HW-8, HW-16, HW-45, HW-28, HW-56, HW-81, HW-97, HW-101, or HW-113. Monoclonal antibodies that are at least 70%, 80%, 90%, 95%, 96%, 97%, 98%, or 99% identical to HW-42, HW-70, HW-25, HW-1, HW-17, HW-8, HW-16, HW-45, HW-28, HW-56, HW-81, HW-97, HW-101, or HW-113 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Monoclonal antibodies having at least one or all six CDR(s) as defined by any conventional definition, e.g., Kabat or IMGT, that are 90%, 95%, 99% or 100% identical to corresponding CDRs of HW-42, HW-70, HW-25, HW-1, HW-17, HW-8, HW-16, HW-45, HW-28, HW-56, HW-81, HW-97, HW-101, or HW-113 are also included.

In some embodiments, the invention provides antibodies, antibody-based binding proteins or antibody fragments thereof that are conservatively modified variants of the anti-PTPRG, anti-ICAM1, anti-CADM1, or anti-GARS antibodies exemplified herein. Typically, the variable regions of these variants have an amino acid sequence that is identical to one of these exemplified sequences except for conservative substitutions at one or more amino acid residues. The invention also provides antibodies having some or all (e.g., 1, 2, 3, 4, 5, and 6) CDRs entirely or substantially from HW-42, HW-70, HW-25, HW-1, HW-17, HW-8, HW-16, HW-45, HW-28, HW-56, HW-81, HW-97, HW-101, or HW-113. Such antibodies can include a heavy chain variable region that has at least two, and usually all three, CDRs entirely or substantially from the heavy chain variable region of HW-42, HW-70, HW-25, HW-1, HW-17, HW-8, HW-16, HW-45, HW-28, HW-56, HW-81, HW-97, HW-101, or HW-113 and/or a light chain variable region having at least two, and usually all three, CDRs entirely or substantially from the light chain variable region of HW-42, HW-70, HW-25, HW-1, HW-17, HW-8, HW-16, HW-45, HW-28, HW-56, HW-81, HW-97, HW-101, or HW-113. The antibodies can include both heavy and light chains. A CDR is substantially from a corresponding HW-42, HW-70, HW-25, HW-1, HW-17, HW-8, HW-16, HW-45, HW-28, HW-56, HW-81, HW-97, HW-101, or HW-113 CDR when it contains no more than 4, 3, 2, or 1 substitutions, insertions, or deletions, except that HCDR2 (when defined by Kabat) can have no more than 6, 5, 4, 3, 2, or 1 substitutions, insertions, or deletions. Such antibodies can have at least 70%, 80%, 90%, 95%, 96%, 97%, 98%, or 99% identity to HW-42, HW-70, HW-25, HW-1, HW-17, HW-8, HW-16, HW-45, HW-28, HW-56, HW-81, HW-97, HW-101, or HW-113 in the amino acid sequence of the mature heavy and/or light chain variable regions and maintain their functional properties, and/or differ from HW-42, HW-70, HW-25, HW-1, HW-17, HW-8, HW-16, HW-45, HW-28, HW-56, HW-81, HW-97, HW-101, or HW-113 by a small number of functionally inconsequential amino acid substitutions (e.g., conservative substitutions), deletions, or insertions.

In some embodiments, the antibodies of the invention comprise heavy chain CDR1, CDR2 and CDR3 sequences and light chain CDR1, CDR2 and CDR3 sequences substantially identical to those of antibodies HW-42, HW-70, HW-25, HW-1, HW-17, HW-8, HW-16, HW-45, HW-28, HW-56, HW-81, HW-97, HW-101, and HW-113. Some antibodies comprise heavy chain CDR1, CDR2 and CDR3 sequences and light chain CDR1, CDR2 and CDR3 sequences identical to those of antibodies HW-42, HW-70, HW-25, HW-1, HW-17, HW-8, HW-16, HW-45, HW-28, HW-56, HW-81, HW-97, HW-101, and HW-113. In some embodiments, the antibodies of the invention have their heavy chain CDR1, CDR2 and CDR3 sequences and their light chain CDR1, CDR2 and CDR3 sequences that are substantially identical to that of the antibodies shown in FIG. 10. The light chain and heavy chain IMGT CDR sequences of the exemplified antibodies are all indicated in the figure. In some of these embodiments, the antibodies have (1) heavy chain CDR1-3 sequences that are substantially identical to SEQ ID NOs:15-17, respectively; and light chain CDR1-3 sequences that are substantially identical to SEQ ID NOs:18-20, respectively; (2) heavy chain CDR1-3 sequences that are substantially identical to SEQ ID NOs:25-27, respectively; and light chain CDR1-3 sequences that are substantially identical to SEQ ID NOs:28-30, respectively; (3) heavy chain CDR1-3 sequences that are substantially identical to SEQ ID NOs:35-37, respectively; and light chain CDR1-3 sequences that are substantially identical to SEQ ID NOs:38-40, respectively; (4) heavy chain CDR1-3 sequences that are substantially identical to SEQ ID NOs:45-47, respectively; and light chain CDR1-3 sequences that are substantially identical to SEQ ID NOs:48-50, respectively; (5) heavy chain CDR1-3 sequences that are substantially identical to SEQ ID NOs:55-57, respectively; and light chain CDR1-3 sequences that are substantially identical to SEQ ID NOs:58-60, respectively; (6) heavy chain CDR1-3 sequences that are substantially identical to SEQ ID NOs:65-67, respectively; and light chain CDR1-3 sequences that are substantially identical to SEQ ID NOs:68-70, respectively; (7) heavy chain CDR1-3 sequences that are substantially identical to SEQ ID NOs:75-77, respectively; and light chain CDR1-3 sequences that are substantially identical to SEQ ID NOs:78-80, respectively; (8) heavy chain CDR1-3 sequences that are substantially identical to SEQ ID NOs:85-87, respectively; and light chain CDR1-3 sequences that are substantially identical to SEQ ID NOs88-90, respectively; (9) heavy chain CDR1-3 sequences that are substantially identical to SEQ ID NOs:95-97, respectively; and light chain CDR1-3 sequences that are substantially identical to SEQ ID NOs:98-100, respectively; (10) heavy chain CDR1-3 sequences that are substantially identical to SEQ ID NOs:105-107, respectively; and light chain CDR1-3 sequences that are substantially identical to SEQ ID NOs:108-110, respectively; (11) heavy chain CDR1-3 sequences that are substantially identical to SEQ ID NOs:115-117, respectively; and light chain CDR1-3 sequences that are substantially identical to SEQ ID NOs118-120, respectively; (12) heavy chain CDR1-3 sequences that are substantially identical to SEQ ID NOs:125-127, respectively; and light chain CDR1-3 sequences that are substantially identical to SEQ ID NOs:128-130, respectively; (13) heavy chain CDR1-3 sequences that are substantially identical to SEQ ID NOs:135-137, respectively; and light chain CDR1-3 sequences that are substantially identical to SEQ ID NOs:138-140, respectively; or heavy chain CDR1-3 sequences that are substantially identical to SEQ ID NOs:145-147, respectively; and light chain CDR1-3 sequences that are substantially identical to SEQ ID NOs:148-150, respectively.

In some embodiments, the antibody of the invention comprises the heavy chain CDR1-CDR3 and light chain CDR1-CDR3 sequences that are respectively identical to the sequences shown in (1) SEQ ID NOs:15-17 and SEQ ID NOs:18-20 (antibody HW-42); (2) SEQ ID NOs:25-27 and SEQ ID NOs:28-30 (antibody HW-70); (3) SEQ ID NOs:35-37 and SEQ ID NOs:38-40 (antibody HW-25); (4) SEQ ID NOs:45-47 and SEQ ID NOs:48-50 (antibody HW-1) (5) SEQ ID NOs:55-57 and SEQ ID NOs:58-60 (antibody HW-17); (6) SEQ ID NOs:65-67 and SEQ ID NOs:68-70 (antibody HW-8); (7) SEQ ID NOs:75-77 and SEQ ID NOs:78-80 (antibody HW-16); (8) SEQ ID NOs:85-87 and SEQ ID NOs:88-90 (antibody HW-45); (9) SEQ ID NOs:95-97 and SEQ ID NOs:98-100 (antibody HW-28); (10) SEQ ID NOs:105-107 and SEQ ID NOs:108-110 (antibody HW-56); (11) SEQ ID NOs:115-117 and SEQ ID NOs:118-120 (antibody HW-81); (12) SEQ ID NOs:125-127 and SEQ ID NOs:128-130 (antibody HW-97); (13) SEQ ID NOs:135-137 and SEQ ID NOs:138-140 (antibody HW-101); or (14) SEQ ID NOs:145-147 and SEQ ID NOs:148-150 (antibody HW-113).

In other embodiments, the antibody of the invention that specifically binds to human PTPRG comprises a mature heavy chain variable region having an amino acid sequence substantially identical to SEQ ID NO:11 and a mature light chain variable region having an amino acid sequence substantially identical to SEQ ID NO:12; a mature heavy chain variable region having an amino acid sequence substantially identical to SEQ ID NO:21 and a mature light chain variable region having an amino acid sequence substantially identical to SEQ ID NO:22; a mature heavy chain variable region having an amino acid sequence substantially identical to SEQ ID NO:31 and a mature light chain variable region having an amino acid sequence substantially identical to SEQ ID NO:32; a mature heavy chain variable region having an amino acid sequence substantially identical to SEQ ID NO:41 and a mature light chain variable region having an amino acid sequence substantially identical to SEQ ID NO:42; or a mature heavy chain variable region having an amino acid sequence substantially identical to SEQ ID NO:51 and a mature light chain variable region having an amino acid sequence substantially identical to SEQ ID NO:52. In some embodiments, the antibody of the invention comprises a mature heavy chain variable region having an amino acid sequence at least 90% identical to SEQ ID NO:11 and a mature light chain variable region having an amino acid sequence at least 90% identical to SEQ ID NO:12; a mature heavy chain variable region having an amino acid sequence at least 90% identical to SEQ ID NO:21 and a mature light chain variable region having an amino acid sequence at least 90% identical to SEQ ID NO:22; a mature heavy chain variable region having an amino acid sequence at least 90% identical to SEQ ID NO:31 and a mature light chain variable region having an amino acid sequence at least 90% identical to SEQ ID NO:32; a mature heavy chain variable region having an amino acid sequence at least 90% identical to SEQ ID NO:41 and a mature light chain variable region having an amino acid sequence at least 90% identical to SEQ ID NO:42; or a mature heavy chain variable region having an amino acid sequence at least 90% identical to SEQ ID NO:51 and a mature light chain variable region having an amino acid sequence at least 90% identical to SEQ ID NO:52. In some embodiments, the percentage identity can be at least 91%, at least 92%, at least 93%, at least 94%, at least 95%, at least 96%, at least 97%, at least 98%, or at least 99%, or 100%. In some antibodies of the invention, the mature heavy chain variable region has an amino acid sequence of SEQ ID NO:11 and the mature light chain variable region has an amino acid sequence of SEQ ID NO:12; the mature heavy chain variable region has an amino acid sequence of SEQ ID NO:21 and the mature light chain variable region has an amino acid sequence of SEQ ID NO:22; the mature heavy chain variable region has an amino acid sequence of SEQ ID NO:31 and the mature light chain variable region has an amino acid sequence of SEQ ID NO:32; the mature heavy chain variable region has an amino acid sequence of SEQ ID NO:41 and the mature light chain variable region has an amino acid sequence of SEQ ID NO:42; or the mature heavy chain variable region has an amino acid sequence of SEQ ID NO:51 and the mature light chain variable region has an amino acid sequence of SEQ ID NO:52.

In other embodiments, the antibody of the invention that specifically binds to human GARS comprises a mature heavy chain variable region having an amino acid sequence substantially identical to SEQ ID NO:61 and a mature light chain variable region having an amino acid sequence substantially identical to SEQ ID NO:62; a mature heavy chain variable region having an amino acid sequence substantially identical to SEQ ID NO:71 and a mature light chain variable region having an amino acid sequence substantially identical to SEQ ID NO:72; a mature heavy chain variable region having an amino acid sequence substantially identical to SEQ ID NO:81 and a mature light chain variable region having an amino acid sequence substantially identical to SEQ ID NO:82; a mature heavy chain variable region having an amino acid sequence substantially identical to SEQ ID NO:91 and a mature light chain variable region having an amino acid sequence substantially identical to SEQ ID NO:92; a mature heavy chain variable region having an amino acid sequence substantially identical to SEQ ID NO:101 and a mature light chain variable region having an amino acid sequence substantially identical to SEQ ID NO:102; or a mature heavy chain variable region having an amino acid sequence substantially identical to SEQ ID NO:111 and a mature light chain variable region having an amino acid sequence substantially identical to SEQ ID NO:112. In some embodiments, the antibody of the invention comprises a mature heavy chain variable region having an amino acid sequence at least 90% identical to SEQ ID NO:61 and a mature light chain variable region having an amino acid sequence at least 90% identical to SEQ ID NO:62; a mature heavy chain variable region having an amino acid sequence at least 90% identical to SEQ ID NO:71 and a mature light chain variable region having an amino acid sequence at least 90% identical to SEQ ID NO:72; a mature heavy chain variable region having an amino acid sequence at least 90% identical to SEQ ID NO:81 and a mature light chain variable region having an amino acid sequence at least 90% identical to SEQ ID NO:82; a mature heavy chain variable region having an amino acid sequence at least 90% identical to SEQ ID NO:91 and a mature light chain variable region having an amino acid sequence at least 90% identical to SEQ ID NO:92; a mature heavy chain variable region having an amino acid sequence at least 90% identical to SEQ ID NO:101 and a mature light chain variable region having an amino acid sequence at least 90% identical to SEQ ID NO:102; or a mature heavy chain variable region having an amino acid sequence at least 90% identical to SEQ ID NO:111 and a mature light chain variable region having an amino acid sequence at least 90% identical to SEQ ID NO:112. In some embodiments, the percentage identity can be at least 91%, at least 92%, at least 93%, at least 94%, at least 95%, at least 96%, at least 97%, at least 98%, or at least 99%, or 100%. In some antibodies of the invention, the mature heavy chain variable region has an amino acid sequence of SEQ ID NO:61 and the mature light chain variable region has an amino acid sequence of SEQ ID NO:62; the mature heavy chain variable region has an amino acid sequence of SEQ ID NO:71 and the mature light chain variable region has an amino acid sequence of SEQ ID NO:72; the mature heavy chain variable region has an amino acid sequence of SEQ ID NO:81 and the mature light chain variable region has an amino acid sequence of SEQ ID NO:82; the mature heavy chain variable region has an amino acid sequence of SEQ ID NO:91 and the mature light chain variable region has an amino acid sequence of SEQ ID NO:92; the mature heavy chain variable region has an amino acid sequence of SEQ ID NO:101 and the mature light chain variable region has an amino acid sequence of SEQ ID NO:102; or the mature heavy chain variable region has an amino acid sequence of SEQ ID NO:111 and the mature light chain variable region has an amino acid sequence of SEQ ID NO:112.

In other embodiments, the antibody of the invention that specifically binds to human CADM1 comprises a mature heavy chain variable region having an amino acid sequence substantially identical to SEQ ID NO:121 and a mature light chain variable region having an amino acid sequence substantially identical to SEQ ID NO:122; or a mature heavy chain variable region having an amino acid sequence substantially identical to SEQ ID NO:131 and a mature light chain variable region having an amino acid sequence substantially identical to SEQ ID NO:132. In some embodiments, the antibody of the invention comprises a mature heavy chain variable region having an amino acid sequence at least 90% identical to SEQ ID NO:121 and a mature light chain variable region having an amino acid sequence at least 90% identical to SEQ ID NO:122; or a mature heavy chain variable region having an amino acid sequence at least 90% identical to SEQ ID NO:131 and a mature light chain variable region having an amino acid sequence at least 90% identical to SEQ ID NO:132. In some embodiments, the percentage identity can be at least 91%, at least 92%, at least 93%, at least 94%, at least 95%, at least 96%, at least 97%, at least 98%, or at least 99%, or even 100%. In some antibodies of the invention, the mature heavy chain variable region has an amino acid sequence of SEQ ID NO:121 and the mature light chain variable region has an amino acid sequence of SEQ ID NO:122, or the mature heavy chain variable region has an amino acid sequence of SEQ ID NO:131 and the mature light chain variable region has an amino acid sequence of SEQ ID NO:132.

In other embodiments, the antibody of the invention that specifically binds to human ICAM1 comprises a mature heavy chain variable region having an amino acid sequence substantially identical to SEQ ID NO:141 and a mature light chain variable region having an amino acid sequence substantially identical to SEQ ID NO:142. In some embodiments, the antibody, antibody fragment, antibody-based binding protein, ADC or CAR of the invention comprises a mature heavy chain variable region having an amino acid sequence at least 90% identical to SEQ ID NO:141 and a mature light chain variable region having an amino acid sequence at least 90% identical to SEQ ID NO:142. In some embodiments, the percentage identity can be at least 91%, at least 92%, at least 93%, at least 94%, at least 95%, at least 96%, at least 97%, at least 98%, or at least 99%, or even 100%. In some antibodies of the invention, the mature heavy chain variable region has an amino acid sequence of SEQ ID NO:141; and the mature light chain variable region has an amino acid sequence of SEQ ID NO:142.

In the various embodiments, percent (%) identity of peptide sequences can be calculated, for example, as 100×[(identical positions)/min(TGA, TGB)], where TGA and TGB are the sum of the number of residues and internal gap positions in peptide sequences A and B in the alignment that minimizes TGA and TGB. See, e.g., Russell et al, J. Mol. Biol., 244: 332-350 (1994).

The antibody, antibody fragment, antibody-based binding protein, ADC or CAR of the invention of the invention can be any antibody including a full length antibody or an antibody fragment that specifically recognizes or binds to a multiple myeloma cell surface antigen, for example PTPRG, ICAM1, CADM1, or GARS. For example, the antibody, antibody fragment or antibody-based binding protein can be polyclonal, monoclonal, recombinant, chimeric, or humanized. Furthermore, the antibody can be of any isotype including without limitation IgA, IgD, IgE, IgG, or IgM. Thus, for example, the antibody can be any IgA such as IgA1 or IgA2, or any IgG such as IgGl, IgG2, IgG3, IgG4, or synthetic IgG. The antibody can also be any antibody fragment or antibody-based binding protein having specificity for the extracellular domain of human ROR2, such as F(ab)2, Fv, scFv, IgGACH2, F(ab′)2, scFv2CH3, Fab, VL, VH, scFv4, scFv3, scFv2, dsFv, Fv, scFv-Fc, (scFv)2, a diabody, and a bivalent antibody. The antibody can be any modified or synthetic antibody, including, but not limited to, non-depleting IgG antibodies, CARs, or other Fc or Fab variants of antibodies.

In addition to a heavy chain as described above, the antibody, antibody-based binding proteins or antibody fragments thereof of the invention can further comprise a light chain selected from a Fab library using sequential naive chain shuffling. Likewise, in addition to a light chain as described above, the antibody of the invention can further comprise a heavy chain selected from a Fab library using sequential naive chain shuffling.

The antibody, antibody-based binding protein or antibody fragment thereof of the invention can be produced by any suitable technique, for example, using any suitable eukaryotic or non-eukaryotic expression system. In certain embodiments, the antibody, antibody-based binding protein or antibody fragment thereof is produced using a mammalian expression system. Some specific techniques for generating the antibodies or antigen-binding fragments of the invention are exemplified herein. In some embodiments, the antibodies or antigen-binding fragments of the invention can be produced using a suitable non-eukaryotic expression system such as a bacterial expression system. Bacterial expression systems can be used to produce fragments such as a F(ab)2, Fv, scFv, IgGACH2, F(ab′)2, scFv2CH3, Fab, VL, VH, scFv4, scFv3, scFv2, dsFv, Fv, scFv-Fc, (scFv)2, and diabodies. Techniques for altering DNA coding sequences to produce such fragments are known in the art.

The antibodies, antibody-based binding proteins or antibody fragments thereof of the invention can be conjugated to a synthetic molecule using any type of suitable conjugation. Recombinant engineering and incorporated selenocysteine (e.g., as described in U.S. Pat. No. 8,916,159 issued on Dec. 23, 2014) can be used to conjugate a synthetic molecule. Other methods of conjugation can include covalent coupling to native or engineered lysine side-chain amines or cysteine side-chain thiols. See, e.g., Wu et al., Nat. Biotechnol, 23: 1 137-1 146 (2005).

In an embodiment, the antibodies, antibody-based binding proteins or antibody fragments thereof of the invention conjugated to a synthetic molecule (called “ADC” for antibody drug conjugate with the synthetic molecule being a toxin) are obtained by means of site-specific chemical or enzymatic antibody conjugation.

The synthetic molecule can be any molecule such as one targeting a tumor. In some embodiments, the synthetic molecule for conjugation to the antibody is a protein (e.g., an antibody) or an RNA or DNA aptamer. In one embodiment, the antibodies, antibody-based binding proteins or antibody fragments thereof of the invention conjugated to a synthetic molecule have the general formula A-(L-P)ₙ, in which: A is an antibody, antibody-based binding protein or antibody fragment thereof as described herein, L is one or more linkers, P is one or more payloads selected from the group consisting of a label and a cytotoxic or cytostatic agent, and in which n is an integer between ≥1 and ≤10. In this embodiment, the linker may comprise, or consists of, at least one selected from the group consisting of: an oligopeptide linker (including cleavable and non-cleavable oligopeptide linkers), a hydrazine linker, a thiourea linker, a self-immolative linker, a succinimidyl trans-4-(maleimidylmethyl)cyclohexane-1-carboxylate (SMCC) linker, a maleimide linker, a disulfide linker, a thioether linker, and/or a maleimide linker.

The skilled person understands that further linkers may be suitable. Such linkers may be non-cleavable or may be cleaved by changes in pH, redox potential or specific intracellular enzymes. Cleavable oligopeptide linkers include protease- or matrix metalloprotease-cleavable linkers. It is understood that the linker may comprise combinations of the above. For example, the linker may be a valine-citrulline PAB linker.

In various embodiments, suitable synthetic molecules (“payloads”) for conjugation to the antibody include, e.g., therapeutic agents such as cytotoxic, cytostatic, or antiangiogenic agents, radioisotopes, and liposomes. A cytotoxic agent can be a plant, fungal, or bacterial molecule. In some embodiments, the cytotoxic agent for conjugation to the antibody of the invention is a small molecular weight toxin (MW<2'000 Dalton, preferably MW<1'000 Dalton), a peptide toxin, or a protein toxin. Many specific examples of these toxins are well known in the art. See, e.g., Dyba et al., Curr. Pharm. Des. 10:2311-34, 2004; Kuyucak et al., Future Med. Chem. 6:1645-58, 2014; Beraud et al., Inflamm. Allergy Drug Targets. 10:322-42, 2011; and Middlebrook et al., Microbiol. Rev. 48:199-221, 1984. In some embodiments, a therapeutic agent is conjugated to the antibody. For example, the therapeutic agent can be a maytansinoid (e.g., maytansinol or DM1 maytansinoid), a taxane, a calicheamicin, a cemadotin, a monomethylauristatin (e.g., monomethylauristatin E or monomethylauristatin F), a pyrrolobenzodiazepine (PBD), an anthracycline, such as a derivative of the highly potent anthracycline PNU-159682. Therapeutic agents also include vincristine and prednisone. In various embodiments, the therapeutic agent that may be employed in the invention can be an antimetabolite (e.g., an antifolate such as methotrexate, a fluoropyrimidine such as 5-fluorouracil, cytosine arabinoside, or an analogue of purine or adenosine); an intercalating agent an intercalating agent (for example, an anthracycline such as doxorubicin, nemorubicine, or preferably a derivative of PNU-159682), daunomycin, epirubicin, idarubicin, mitomycin-C, dactinomycin, or mithramycin, or other intercalating agents such as pyrrolobenzodiazepine); a DNA-reactive agent such as calicheamicins, tiancimycins, and other enediynes; a platinum derivative (e.g., cisplatin or carboplatin); an alkylating agent (e.g., nitrogen mustard, melphalan, chlorambucil, busulphan, cyclophosphamide, ifosfamide nitrosoureas or thiotepa); an RNA polymerase inhibitor such as α-amanitin; an antimitotic agent (e.g., a vinca alkaloid such as vincristine, or a taxoid such as paclitaxel or docetaxel); a topoisomerase inhibitor (for example, etoposide, teniposide, amsacrine, topotecan); a cell cycle inhibitor (for example, a flavopyridol); or a microtubule agent (e.g., an epothilone, a tubulysine, a pre-tubulysine, a discodermolide analog, or an eleutherobin analog). A therapeutic agent can be a proteosome inhibitor or a topoisomerase inhibitor such as bortezomib, amsacrine, etoposide, etoposide phosphate, teniposide, or doxorubicin. Therapeutic radioisotopes include iodine (¹³¹I), yttrium (⁹⁰Y), lutetium (¹⁷⁷Lu), actinium (²²⁵Ac), praseodymium, astatine (At), rhenium (Re), bismuth (Bi or Bi), and rhodium (Rh). Antiangiogenic agents include linomide, bevacuzimab, angiostatin, and razoxane.

In some embodiments, the synthetic molecule can be conjugated to any antibody, antibody-based binding protein, or antibody-fragment. In some embodiments, the synthetic molecule can be a label. Labels can be useful in diagnostic applications and can include, for example, contrast agents. A contrast agent can be a radioisotope label such as iodine (¹³¹I or ¹²⁵I), indium (¹¹¹In), technetium (⁹⁹Tc), phosphorus (³²P), carbon (¹⁴C), tritium (³H), other radioisotope (e.g., a radioactive ion), or a therapeutic radioisotope such as one of the therapeutic radioisotopes listed above. Additionally, contrast agents can include radiopaque materials, magnetic resonance imaging (MRI) agents, ultrasound imaging agents, and any other contrast agents suitable for detection by a device that images an animal body. A synthetic molecule can also be a fluorescent label, a biologically active enzyme label, a luminescent label, or a chromophore label.

In some other embodiments, the synthetic molecule can be a liposome, as described in Bendas, BioDrugs, 15: 215-224, 2001. In such embodiments, the antibody can be conjugated to a colloidal particle, e.g., a liposome, and used for controlled delivery of an agent to diseased cells. In preparing an antibody conjugated to a liposome, e.g., an immunoliposome, an agent such as a chemotherapeutic or other drug can be entrapped in the liposome for delivery to a target cell. In some embodiments, the antibodies, antibody-based binding proteins or antibody fragments thereof of the invention can also have specificity for one or more antigens in addition to PTPRG, ICAM1, CADM1, or GARS. For example, the antibody of the invention can be engineered (e.g., as a bivalent diabody or a conjugated Fab dimer or trimer) to have specificity for PTPRG, ICAM1, CADM1, or GARS and another tumor antigen, e.g., an antigen associated with a hematologic malignancy, for example multiple myeloma or acute myeloid leukemia (AML). The antibody can be engineered to have specificity for PTPRG, ICAM1, CADM1, or GARS and an antigen that promotes activation or targeting of cytotoxic effector cells.

### V. Polynucleotides, Vectors and Host Cells for Producing Anti-PTPRG, Anti-ICAM1, Anti-CADM1, or Anti-GARS Antibodies

The invention provides substantially purified polynucleotides (DNA or RNA) that are identical or complementary to sequences encoding polypeptides comprising segments or domains of the antibody, antibody-based binding protein or antibody fragment thereof chains described herein. In some embodiments, the polynucleotides of the invention encode the heavy chain or light chain domains sequences shown in FIG. 10. When expressed from appropriate expression vectors, polypeptides encoded by these polynucleotides are capable of exhibiting PTPRG, ICAM1, CADM1, or GARS antigen binding capacity.

Also provided in the invention are polynucleotides which encode at least one CDR region and usually all three CDR regions from the heavy or light chain of the antibodies described herein. Some other polynucleotides encode all or substantially all of the variable region sequence of the heavy chain and/or the light chain of the exemplified antibodies. For example, some of these polynucleotides encode the amino acid sequence of the heavy chain variable region shown in any one SEQ ID NOs:11, 21, 31, 41, 51, 61, 71, 81, 91, 101, 111, 121, 131, and 141, and/or the amino acid sequence of the light chain variable region shown in any one SEQ ID NOs:12, 22, 32, 42, 52, 62, 72, 82, 92, 102, 112, 122, 132, and 142. Because of the degeneracy of the code, a variety of nucleic acid sequences will encode each of the immunoglobulin amino acid sequences.

The polynucleotides of the invention can encode only the variable region sequences of the exemplified antibodies. They can also encode both a variable region and a constant region of the antibody. Some of polynucleotide sequences of the invention nucleic acids encode a mature heavy chain variable region sequence that is substantially identical (e.g., at least 80%, 90%, 95% or 99%) to the mature heavy chain variable region sequence shown in any one SEQ ID NOs: 11, 21, 31, 41, 51, 61, 71, 81, 91, 101, 111, 121, 131, and 141. Some other polynucleotide sequences encode a mature light chain variable region sequence that is substantially identical to the mature light chain variable region sequence shown in any one SEQ ID NOs: 12, 22, 32, 42, 52, 62, 72, 82, 92, 102, 112, 122, 132, and 142. Some of the polynucleotide sequences encode a polypeptide that comprises variable regions of both the heavy chain and the light chain of one of the exemplified antibodies. Some other polynucleotides encode two polypeptide segments that respectively are substantially identical to the variable regions of the heavy chain or the light chain of one of the exemplified antibodies.

The polynucleotide sequences can be produced by de novo solid-phase DNA synthesis or by PCR mutagenesis of an existing sequence (e.g., sequences as described in the Examples below) encoding an exemplified functional antibody. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991.

Also provided in the invention are expression vectors and host cells for producing the functional antibodies described herein. Specific examples of plasmid and transposon based vectors for expressing the antibodies are described in the Examples below. Various other expression vectors can also be employed to express the polynucleotides encoding the functional antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the antibody polynucleotides and polypeptides in mammalian (e.g., human) cells include pCEP4, pREP4, pThioHis A, B & C, pcDNA3.1/His, pEBVHis A, B & C (Invitrogen, San Diego, CA), MPSV vectors, and numerous other vectors known in the art for expressing other proteins. Other useful nonviral vectors comprise expression cassettes that can be mobilized with Sleeping Beauty, PiggyBack and other transposon systems. Useful viral vectors include vectors based on lentiviruses or other retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding a functional antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of a functional antibody chain or fragment. These elements typically include an ATG initiation codon and adjacent ribosome binding site (Kozak consensus sequence) or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted functional antibody sequences. More often, the inserted functional antibody sequences are linked to a signal sequences before inclusion in the vector. Vectors to be used to receive sequences encoding the functional antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human, and preferably of human IgG1 antibodies. Exemplary chimeric full-length antibodies of the rabbit variable domains fused to the constant region domains of human IgG₁ antibodies comprise a heavy chain of SEQ ID NO:13 and a light chain of SEQ ID NO:14 (antibody HW-42); a heavy chain of SEQ ID NO:23 and a light chain of SEQ ID NO:24 (antibody HW-70); a heavy chain of SEQ ID NO:33 and a light chain of SEQ ID NO:34 (antibody HW-25); a heavy chain of SEQ ID NO:43 and a light chain of SEQ ID NO:44 (antibody HW-1); a heavy chain of SEQ ID NO:53 and a light chain of SEQ ID NO:54 (antibody HW-17); a heavy chain of SEQ ID NO:63 and a light chain of SEQ ID NO:64 (antibody HW-8); a heavy chain of SEQ ID NO:73 and a light chain of SEQ ID NO:74 (antibody HW-16); a heavy chain of SEQ ID NO:83 and a light chain of SEQ ID NO:84 (antibody HW-45); a heavy chain of SEQ ID NO:93 and a light chain of SEQ ID NO:94 (antibody HW-28); a heavy chain of SEQ ID NO:103 and a light chain of SEQ ID NO:104 (antibody HW-56); a heavy chain of SEQ ID NO:113 and a light chain of SEQ ID NO:114 (antibody HW-81); a heavy chain of SEQ ID NO:123 and a light chain of SEQ ID NO:124 (antibody HW-97); a heavy chain of SEQ ID NO:133 and a light chain of SEQ ID NO:134 (antibody HW-101); and a heavy chain of SEQ ID NO:143 and a light chain of SEQ ID NO:144 (antibody HW-113).

The host cells for harboring and expressing the functional antibody chains can be either prokaryotic or eukaryotic. In some preferred embodiments, mammalian host cells are used to express and to produce the antibody polypeptides of the present invention. For example, they can be either a hybridoma cell line expressing endogenous immunoglobulin genes or a mammalian cell line harboring an exogenous expression vector. These include any normal mortal or normal or abnormal immortal animal or human cell. In addition to the cell lines exemplified herein, a number of other suitable host cell lines capable of secreting intact immunoglobulins are also known in the art. These include, e.g., the CHO cell lines, various HEK 293 cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer, and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, EF1α and human UbC promoters exemplified herein, the metallothionein promoter, the constitutive adenovirus major late promoter, the dexamethasone-inducible MMTV promoter, the SV40 promoter, the MRP pol III promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transformation is commonly utilized for prokaryotic cells, whereas calcium phosphate treatment or electroporation may be used for other cellular hosts (see generally Brent et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate for the cell type.

The invention further provides eukaryotic or non-eukaryotic cells (e.g., T lymphocytes) that have been recombinantly engineered to produce the antibodies, antibody-based binding proteins or antibody fragments thereof of the invention. The eukaryotic or non-eukaryotic cells can be used as an expression system to produce the antibody of the invention. In some embodiments, the invention provides PTPRG, ICAM1, CADM1, or GARS targeted immune cells that are engineered to recombinantly express an PTPRG, ICAM1, CADM1, or GARS specific antibody of the invention. For example, the invention provides a T cell or NK cell engineered to express an antibody of the invention (e.g., an scFv, scFv-Fc, or (scFv)2), which is linked to a synthetic molecule containing one or more of the following domains: a spacer or hinge region (e.g., a CD28 sequence or a IgG4 hinge-Fc sequence), a transmembrane region (e.g., a transmembrane canonical domain), and an intracellular T-cell receptor (TCR) signaling domain, thereby forming a chimeric antigen receptor (CAR) or T-body. Intracellular TCR signaling domains that can be included in a CAR (or T-body) include, but are not limited to, CD3ζ, FcR-γ, and Syk-PT signaling domains as well as the CD28, 4-1BB, and CD134 co-signaling domains. Methods for constructing T cells and NK cells expressing a CAR are known in the art. See, e.g., Marcu-Malina et al., Expert Opinion on Biological Therapy, Vol. 9, No. 5 (posted online on Apr. 16, 2009) and Lu H, Zhao X, Li Z, Hu Y, Wang H. Front Oncol. 2021 Aug. 6; 11:720501.

### VI. Therapeutic and Diagnostic Applications

In one aspect, the invention provides methods for inhibiting cells that express PTPRG, ICAM1, CADM1, or GARS (PTPRG, ICAM1, CADM1, or GARS cells, respectively) by contacting the cells with an antibody, antibody-based binding protein or antibody fragment thereof of the invention, or an antibody drug conjugate (ADC) or an engineered cell harboring a chimeric antigen receptor (CAR) described herein. The antibody, antibody-based binding protein or antibody fragment thereof can be a naked (unconjugated) molecule or an antibody molecule conjugated to a synthetic molecule, e.g., a cytotoxic, cytostatic, or antiangiogenic agent, a radioisotope, or even to a liposome. The method can be used to inhibit PTPRG, ICAM1, CADM1, or GARS cells in vitro or in a subject (i.e., in vivo). The contacted PTPRG, ICAM1, CADM1, or GARS cells can be in, for example, a cell culture or animal model of a disorder associated with elevated levels of PTPRG, ICAM1, CADM1, or GARS. The methods are useful, for example, to measure and/or rank (relative to another antibody) the antibody's inhibitory activity for a specific PTPRG, ICAM1, CADM1, or GARS cell type. Inhibiting PTPRG, ICAM1, CADM1, or GARS cells can include blocking or reducing the activity or growth of PTPRG, ICAM1, CADM1, or GARS cells. Inhibiting can also include the killing of PTPRG, ICAM1, CADM1, or GARS cells. Inhibitory activity can also be mediated by recruitment of immune system effectors that attack PTPRG, ICAM1, CADM1, or GARS, e.g., by activating constituents of the antibody-dependent cell-mediated cytotoxicity (ADCC) or complement systems.

In some related embodiments, the invention provides methods for treating a subject that has, is suspected to have, or is at risk of developing a disorder associated with elevated levels of PTPRG, ICAM1, CADM1, or GARS. Generally, the methods include administering a pharmaceutical composition that contains a therapeutically effective amount of an isolated antibody, antibody-based binding protein, antibody fragment thereof, ADC or CAR of the invention to the subject. The antibody can be any anti-PTPRG, ICAM1, CADM1, or GARS antibody of the invention as described herein. Thus, the antibody can be chimeric, humanized, synthetic, F(ab)2, Fv, scFv, IgGACH2, F(ab′)2, scFv2CH3, Fab, VL, VH, scFv4, scFv3, scFv2, dsFv, Fv, or (scFv)2. In some embodiments, the method includes administering an IgG, an scFv, a dsFv, a F(ab′)2, a diabody, or a bivalent antibody. The administered antibody or antigen-binding fragment can be conjugated to a synthetic molecule described above, e.g., a cytotoxic, cytostatic, or antiangiogenic agent, a therapeutic radioisotope, or a liposome. An exemplary cytotoxic agent is Pseudomonas exotoxin A (PE38). Disorders that can be treated include a hematologic malignancy, for example MM or AML and other disorders with elevated PTPRG, ICAM1, CADM1, or GARS expression.

In some embodiments, the invention provides methods for treating a subject that has, is suspected to have, or is at risk of developing a disorder associated with expression of PTPRG, ICAM1, CADM1, or GARS by adoptive transfer of the genetically engineered T-cells described herein, which express an antibody or antigen-binding fragment of the invention as a chimeric antigen receptor (CAR) that selectively binds PTPRG, ICAM1, CADM1, or GARS. Recombinant technology can be used to introduce CAR-encoding genetic material into any suitable T cells, e.g., autologous T cells from the subject to be treated, off-the-shelf allogeneic T cells, or autologous or off-the-shelf allogeneic NK cells. The T or NK cells carrying the genetic material can be expanded (e.g., in the presence of cytokines). The genetically engineered T or NK cells are transferred, typically by infusion, to the patient. The transferred T or NK cells of the invention can then mount an immune response against PTPRG, ICAM1, CADM1, or GARS expressing cells in the subject. The adoptive transfer method can be used, for example, to treat subjects that have or are suspected to have a hematologic malignancy, for example MM or AML.

In some embodiments, the foregoing methods of treatment can further include co-administering a second therapeutic agent for treating the disorder associated with elevated PTPRG, ICAM1, CADM1, or GARS. For example, when the disorder to be treated involves an PTPRG, ICAM1, CADM1, or GARS-expressing hematologic malignancy, the method can further include co-administration of a cytotoxic, cytostatic, or antiangiogenic or immune-stimulatory agent (e.g. immune-checkpoint inhibitor antibodies, for instance, but not limited to, those binding to PD1, PDL1, CTLA4, OX40, TIM3, GITR, LAG3 and the like) suitable for treating the hematologic malignancy. If the hematologic malignancy is a B-cell malignancy, the method can further include, for example, co-administration of rituximab, alemtuzumab, ofatumumab, ocrelizumab, a BTK or BCL2 inhibitor, or a CHOP chemotherapeutic regimen. If the hematologic malignancy is MM, the method can further include, for example, co-administration of chemotherapy, bortezomib, daratumumab, isatuximab, elotuzumab, lenalidomide, idecabtagene vicleucel, or ciltacabtagene autoleucel. If the hematologic malignancy is AML, the method can further include, for example, co-administration of chemotherapy, venetoclax, or gemtuzumab ozogamicin.

In some other embodiments, the invention provides method for detecting in a biological sample an altered level of PTPRG, ICAM1, CADM1, or GARS (e.g., cell surface PTPRG, ICAM1, CADM1, or GARS), for example, relative to a control, either by FACS, immunohistochemistry (IHC) or Western Blotting. Generally, the method includes contacting a biological sample with an antibody, antibody-based binding protein, antibody fragment thereof of the invention and determining the amount of antibody that selectively binds to material (e.g., cells) in the sample to thereby determine the level of PTPRG, ICAM1, CADM1, or GARS in the biological sample. A biological sample can be from a cell culture or from a test subject, e.g., a plasma or a tissue sample from a subject that has, is suspected to have, or is at risk of developing a disease or condition associated with elevated PTPRG, ICAM1, CADM1, or GARS in a subject. A control level desirably corresponds to the PTPRG, ICAM1, CADM1, or GARS level detected using the same antibody in a corresponding sample(s) from one or more control cultures or disease-free subjects. Methods of using the antibody of the invention to determine PTPRG, ICAM1, CADM1, or GARS levels can include any immunoassay such as immuno- (Western) blotting, enzyme-linked immunosorbent assay (ELISA), Immunohistochemistry (IHC) and flow cytometry, e.g., fluorescence-activated cell sorting (FACS) analysis.

The methods of detection can be used to screen for the presence of a disorder associated with elevated PTPRG, ICAM1, CADM1, or GARS. The methods include obtaining a sample from a test subject in need of screening, e.g., a subject that has, is suspected to have, or is at risk of developing a disorder associated with elevated PTPRG, ICAM1, CADM1, or GARS. The level of PTPRG, ICAM1, CADM1, or GARS (e.g., the amount or concentration) in the sample is measured using an antibody, antibody-based binding protein, antibody fragment thereof of the invention, and the level in the sample is compared to a control level of PTPRG, ICAM1, CADM1, or GARS. The control level represents, for example, the mean level (e.g., the amount or concentration) in sample(s) from one or, preferably, multiple control group subjects that do not have a disorder associated with elevated PTPRG, ICAM1, CADM1, or GARS. Alternatively, the control level can correspond to the level or mean level of PTPRG, ICAM1, CADM1, or GARS in one or more samples taken from the test subject at one or more prior times, such as when the test subject did not have or did not exhibit, a condition associated with elevated PTPRG, ICAM1, CADM1, or GARS. A significantly higher level of PTPRG, ICAM1, CADM1, or GARS in the biological sample relative to the control level is indicative of a disorder associated with elevated PTPRG, ICAM1, CADM1, or GARS in the subject.

Additionally, the methods of detection can be used to monitor the progress of a disorder associated with elevated PTPRG, ICAM1, CADM1, or GARS. The method includes obtaining a sample from a subject in need of screening, e.g., a subject having been diagnosed or suspected to have a disorder associated with elevated PTPRG, ICAM1, CADM1, or GARS. The level of PTPRG, ICAM1, CADM1, or GARS in the sample is measured using an antibody, antibody-based binding protein, antibody fragment thereof of the invention, and the level in the sample is compared to a control level corresponding to the level or mean level of PTPRG, ICAM1, CADM1, or GARS in one or more samples taken from the test subject at one or more prior times. Levels of PTPRG, ICAM1, CADM1, or GARS that are significantly elevated or decreased relative to control indicate that the subject's disorder is deteriorating or improving, respectively. The foregoing methods of detection can be used to screen for the presence or to monitor the progress of disorders including, for example, a hematologic malignancy, for example MM or AML.

In some embodiments, the invention provides methods for screening a subject for an altered level of PTPRG, ICAM1, CADM1, or GARS. Generally, the methods entail administering to the subject an antibody, antibody-based binding protein, antibody fragment thereof of the invention that is conjugated to a label (e.g., a contrast agent), imaging the subject in a manner suitable for detecting the label, and determining whether a region in the subject has an altered density or concentration of label as compared to the background level of label in proximal tissue. Alternatively, the methods include determining whether there is an altered density or concentration of label in a region as compared to the density or concentration of label previously detected in the same region of the subject. Methods of imaging a subject can include x-ray imaging, x-ray computed tomography (CT) imaging (e.g., CT angiography (CTA) imaging), magnetic resonance (MR) imaging, magnetic resonance angiography (MRA), nuclear medicine, ultrasound (US) imaging, optical imaging, elastography, infrared imaging, microwave imaging, and the like, as appropriate for detecting the label conjugated to the antibody. In a preferred embodiment, the subject has, is suspected to have, or is at risk of developing an PTPRG, ICAM1, CADM1, or GARS-expressing tumor, such as a hematologic malignancy, for example MM or AML, and the method is used to screen for or detect the presence of the tumor. In another embodiment, the method can be used to monitor the size or density of a PTPRG, ICAM1, CADM1, or GARS-expressing tumor over time, e.g., during a course of treatment.

### VII. Pharmaceutical Compositions and Combinations

In another aspect, the invention provides pharmaceutical compositions that contain an antibody, an antibody fragment, an antibody-based binding protein, or an ADC as described herein and a pharmaceutically acceptable carrier. Pharmaceutical compositions can be prepared from any of the antibodies or related compounds described herein. Exemplary compositions include one or more of an antibody comprising a mature heavy chain variable region having an amino acid sequence of SEQ ID NO:11 and a mature light chain variable region having an amino acid sequence of SEQ ID NO:12; a mature heavy chain variable region having an amino acid sequence of SEQ ID NO:21 and a mature light chain variable region having an amino acid sequence of SEQ ID NO:22; a mature heavy chain variable region having an amino acid sequence of SEQ ID NO:31 and a mature light chain variable region having an amino acid sequence of SEQ ID NO:32; a mature heavy chain variable region having an amino acid sequence of SEQ ID NO:41 and a mature light chain variable region having an amino acid sequence of SEQ ID NO:42; a mature heavy chain variable region having an amino acid sequence of SEQ ID NO:51 and a mature light chain variable region having an amino acid sequence of SEQ ID NO:52, a mature heavy chain variable region having an amino acid sequence of SEQ ID NO:61 and a mature light chain variable region having an amino acid sequence of SEQ ID NO:62; a mature heavy chain variable region having an amino acid sequence of SEQ ID NO:71 and a mature light chain variable region having an amino acid sequence of SEQ ID NO:72; a mature heavy chain variable region having an amino acid sequence of SEQ ID NO:81 and a mature light chain variable region having an amino acid sequence of SEQ ID NO:82; a mature heavy chain variable region having an amino acid sequence of SEQ ID NO:91 and a mature light chain variable region having an amino acid sequence of SEQ ID NO:92; a mature heavy chain variable region having an amino acid sequence of SEQ ID NO:101 and a mature light chain variable region having an amino acid sequence of SEQ ID NO:102; a mature heavy chain variable region having an amino acid sequence of SEQ ID NO:111 and a mature light chain variable region having an amino acid sequence of SEQ ID NO:112, a mature heavy chain variable region having an amino acid sequence of SEQ ID NO:121 and a mature light chain variable region having an amino acid sequence of SEQ ID NO:122; a mature heavy chain variable region having an amino acid sequence of SEQ ID NO:131 and a mature light chain variable region having an amino acid sequence of SEQ ID NO:132, a mature heavy chain variable region having an amino acid sequence of SEQ ID NO:141 and a mature light chain variable region having an amino acid sequence of SEQ ID NO:142.

Other exemplary compositions of the invention can contain an antibody having one, two, three, four, five, or six CDRs selected from the group consisting of SEQ ID NOs:15-17 and SEQ ID NOs:18-20. Other exemplary compositions of the invention can contain an antibody having one, two, three, four, five, or six CDRs selected from the group consisting of SEQ ID NOs:25-27 and SEQ ID NOs:28-30. Other exemplary compositions of the invention can contain an antibody having one, two, three, four, five, or six CDRs selected from the group consisting of SEQ ID NOs:35-37 and SEQ ID NOs:38-40. Other exemplary compositions of the invention can contain an antibody having one, two, three, four, five, or six CDRs selected from the group consisting of SEQ ID NOs:45-47 and SEQ ID NOs:48-50. Other exemplary compositions of the invention can contain an antibody having one, two, three, four, five, or six CDRs selected from the group consisting of SEQ ID NOs:55-57 and SEQ ID NOs:58-60. Other exemplary compositions of the invention can contain an antibody having one, two, three, four, five, or six CDRs selected from the group consisting of SEQ ID NOs:65-67 and SEQ ID NOs:68-70. Other exemplary compositions of the invention can contain an antibody having one, two, three, four, five, or six CDRs selected from the group consisting of SEQ ID NOs:75-77 and SEQ ID NOs:78-80. Other exemplary compositions of the invention can contain an antibody having one, two, three, four, five, or six CDRs selected from the group consisting of SEQ ID NOs:85-87 and SEQ ID NOs:88-90. Other exemplary compositions of the invention can contain an antibody having one, two, three, four, five, or six CDRs selected from the group consisting of SEQ ID NOs:95-97 and SEQ ID NOs:98-100. Other exemplary compositions of the invention can contain an antibody having one, two, three, four, five, or six CDRs selected from the group consisting of SEQ ID NOs:105-107 and SEQ ID NOs:108-110. Other exemplary compositions of the invention can contain an antibody having one, two, three, four, five, or six CDRs selected from the group consisting of SEQ ID NOs:115-117 and SEQ ID NOs:118-120. Other exemplary compositions of the invention can contain an antibody having one, two, three, four, five, or six CDRs selected from the group consisting of SEQ ID NOs:125-127 and SEQ ID NOs:128-130. Other exemplary compositions of the invention can contain an antibody having one, two, three, four, five, or six CDRs selected from the group consisting of SEQ ID NOs:135-137 and SEQ ID NOs:138-140. Other exemplary compositions of the invention can contain an antibody having one, two, three, four, five, or six CDRs selected from the group consisting of (14) SEQ ID NOs:145-147 and SEQ ID NOs:148-150. In some embodiments, however, the antibody includes three CDR sequences of the exemplified light or heavy chains shown in FIG. 10. These include the heavy chain CDR1, CDR2 and CDR3 sequences and light chain CDR1, CDR2 and CDR3 sequences respectively shown in (1) SEQ ID NOs:15-17 and SEQ ID NOs:18-20 (antibody HW-42); (2) SEQ ID NOs:25-27 and SEQ ID NOs:28-30 (antibody HW-70); (3) SEQ ID NOs:35-37 and SEQ ID NOs:38-40 (antibody HW-25); (4) SEQ ID NOs:45-47 and SEQ ID NOs:48-50 (antibody HW-1) (5) SEQ ID NOs:55-57 and SEQ ID NOs:58-60 (antibody HW-17); (6) SEQ ID NOs:65-67 and SEQ ID NOs:68-70 (antibody HW-8); (7) SEQ ID NOs:75-77 and SEQ ID NOs:78-80 (antibody HW-16); (8) SEQ ID NOs:85-87 and SEQ ID NOs:88-90 (antibody HW-45); (9) SEQ ID NOs:95-97 and SEQ ID NOs:98-100 (antibody HW-28); (10) SEQ ID NOs:105-107 and SEQ ID NOs:108-110 (antibody HW-56); (11) SEQ ID NOs:115-117 and SEQ ID NOs:118-120 (antibody HW-81); (12) SEQ ID NOs:125-127 and SEQ ID NOs:128-130 (antibody HW-97); (13) SEQ ID NOs:135-137 and SEQ ID NOs:138-140 (antibody HW-101); or (14) SEQ ID NOs:145-147 and SEQ ID NOs:148-150 (antibody HW-113). In some embodiments, the pharmaceutical composition includes an antibody having six CDR sequences of the same antibody exemplified in FIG. 10, e.g., (1) SEQ ID NOs:15-17 and SEQ ID NOs:18-20 (antibody HW-42); (2) SEQ ID NOs:25-27 and SEQ ID NOs:28-30 (antibody HW-70); (3) SEQ ID NOs:35-37 and SEQ ID NOs:38-40 (antibody HW-25); (4) SEQ ID NOs:45-47 and SEQ ID NOs:48-50 (antibody HW-1) (5) SEQ ID NOs:55-57 and SEQ ID NOs:58-60 (antibody HW-17); (6) SEQ ID NOs:65-67 and SEQ ID NOs:68-70 (antibody HW-8); (7) SEQ ID NOs:75-77 and SEQ ID NOs:78-80 (antibody HW-16); (8) SEQ ID NOs:85-87 and SEQ ID NOs:88-90 (antibody HW-45); (9) SEQ ID NOs:95-97 and SEQ ID NOs:98-100 (antibody HW-28); (10) SEQ ID NOs:105-107 and SEQ ID NOs:108-110 (antibody HW-56); (11) SEQ ID NOs:115-117 and SEQ ID NOs:118-120 (antibody HW-81); (12) SEQ ID NOs:125-127 and SEQ ID NOs:128-130 (antibody HW-97); (13) SEQ ID NOs:135-137 and SEQ ID NOs:138-140 (antibody HW-101); or (14) SEQ ID NOs:145-147 and SEQ ID NOs:148-150 (antibody HW-113). Still another exemplary pharmaceutical composition includes a dsFv fragment, which can include one or more modifications to the amino acid sequence as appropriate and understood by one of ordinary skill in the art.

In some embodiments, the compositions of the invention contain a carrier for the antibody, the antibody fragment, the antibody-based binding protein or the ADC, desirably a pharmaceutically acceptable carrier. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier). The term “carrier” denotes an organic or inorganic ingredient, natural or synthetic, with which the active ingredient is combined to facilitate the use of the active ingredient, e.g., the administration of the active ingredient to a subject. The pharmaceutically acceptable carrier can be co-mingled with one or more of the active components, e.g., a hybrid molecule, and with each other, when more than one pharmaceutically acceptable carrier is present in the composition, in a manner so as not to substantially impair the desired pharmaceutical efficacy. Pharmaceutically acceptable materials typically are capable of administration to a subject, e.g., a patient, without the production of significant undesirable physiological effects such as nausea, dizziness, rash, or gastric upset. It is, for example, desirable for a composition comprising a pharmaceutically acceptable carrier not to be immunogenic when administered to a human patient for therapeutic purposes.

Pharmaceutical compositions of the invention can additionally contain suitable buffering agents, including, for example, acetic acid in a salt, citric acid in a salt, boric acid in a salt, and phosphoric acid in a salt. The compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

A composition suitable for parenteral administration conveniently comprises a sterile aqueous preparation of the inventive composition, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found in Remington's Pharmaceutical Sciences, Mack Publishing Co., Easton, PA.

Preparation of pharmaceutical compositions of the invention and their various routes of administration can be carried out in accordance with methods well known in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. The delivery systems useful in the context of the invention include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and triglycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

The invention also provides kits suitable for carrying out the methods of the invention. Typically, the kits contain two or more components required for performing the therapeutic or diagnostic methods of the invention. Kit components include, but are not limited to, one or more antibodies, antibody-based binding proteins, antibody fragments thereof, or ADCs of the invention, appropriate reagents, and/or equipment. In some embodiments, the kits can contain an antibody, antibody-based binding protein, antibody fragment thereof, or ADC of the invention and an immunoassay buffer suitable for detecting PTPRG, ICAM1, CADM1, or GARS (e.g. by ELISA, flow cytometry, magnetic sorting, or FACS). The kit may also contain one or more microtiter plates, standards, assay diluents, wash buffers, adhesive plate covers, magnetic beads, magnets, and/or instructions for carrying out a method of the invention using the kit. The kit scan include an antibody, antibody-based binding protein, antibody fragment thereof of the invention bound to a substrate (e.g., a multi-well plate or a chip), which is suitably packaged and useful to detect PTPRG, ICAM1, CADM1, or GARS. In some embodiments, the kits include an antibody, antibody-based binding protein, antibody fragment thereof of the invention that is conjugated to a label, such as, a fluorescent label, a biologically active enzyme label, a luminescent label, or a chromophore label. The kits can further include reagents for visualizing the conjugated antibody, antibody-based binding protein, antibody fragment thereof, e.g., a substrate for the enzyme. In some embodiments, the kits include an antibody or antigen-binding fragment of the invention that is conjugated to a contrast agent and, optionally, one or more reagents or pieces of equipment useful for imaging the antibody, antibody-based binding protein, antibody fragment thereof in a subject.

Generally the antibody, antibody-based binding protein, antibody fragment thereof or ADC of the invention in a kit is suitably packaged, e.g., in a vial, pouch, ampoule, and/or any container appropriate for a therapeutic or detection method. Kit components can be provided as concentrates (including lyophilized compositions), which may be further diluted prior to use, or they can be provided at the concentration of use. For use of the antibody of the invention in vivo, single dosages may be provided in sterilized containers having the desired amount and concentration of components.

### EXAMPLES

The following examples are provided to further illustrate the invention but not to limit its scope. Other variants of the invention will be readily apparent to one of ordinary skill in the art and are encompassed by the appended claims.

### Example 1. Development of FBC-Seq

To focus our antibody discovery efforts on tumor-associated cell surface epitopes, a comparative WCP and NGS approach was developed, termed FBC-seq. This method builds upon a previously established WCP strategy, FBC, which augments selection efficiency via bald phage depletion [16]. For FBC-seq, a Fab-phage library is split and selected in parallel on distinct cell types in a single panning round (FIG. 1A). To identify low abundance clones unlikely to be identified using conventional screening approaches, NGS was chosen for analysis of the panning outputs. Numerous bioinformatic tools exist for NGS analysis of phage antibody repertoires, including clonal clustering programs such as CD-HIT (68) or UCLUST (69), and programs that identify immunoglobulin germlines, including IgBLAST (70) and IMGT/High V-QUEST (71). Furthermore, publicly available programs are available to differentially analyze antibody repertoires, such as IMGT/StatClonotype, (72) however, these are mostly useful for V, D, or J gene usage comparisons, not identifying clones via HCDR3. Thus a stand-alone streamlined bioinformatic approach was developed to facilitate reproducibility. To prepare panning outputs for NGS analysis, VH domains are first amplified using primers specific for heavy chain constant domain (CH1) and the pelB signal sequence of the pC3C phagemid. Multiplexed NGS of the heavy chain complementarity determining region 3 (HCDR3) is conducted via single-end MiSeq, followed by in silico HCDR3 extraction and counting (FIG. 1B). A nested PCR approach was employed to incorporate the Illumina P5 and P7 cluster forming sites, as well as barcoded indices to enable parallel sequencing of replicate phage repertoires. Single-end 150-cycle sequencing is then carried out, which spans the HCDR3. HCDR3 sequences, per the IMGT definition, (73) are extracted from demultiplexed sequencing libraries and their abundance compiled for each panning replicate. After this, HCDR3 differential abundance is determined between the distinct panning conditions. Conserved VH and VJ rabbit germline encoded sequences flanking the HCDR3 are detected using regular expression (Regex) patterns and used as splice sites for HCDR3 extraction. (74) For differential abundance analysis of the sequenced panning repertoires, DESeq2, a well-established method for counting applications such as RNA-seq or ChIP-seq, is called upon. (75) DESeq2 generates differential abundance calculations and statistical analyses for every HCDR3 identifier detected, among other information, which is used to distinguish mAbs on the basis of differential specificity. Differential abundance analysis (DESeq2) then identifies selectively enriched HCDR3s between the distinct panning conditions (FIG. 1C), which are hypothesized to barcode mAbs that recognize differentially enriched surface epitopes. We reasoned that this HCDR3-focused approach would provide sufficient identification of antibody uniqueness, as the HCDR3 is the highest diversity region of the antibody and is typically a crucial component of the paratope. Moreover, antibody clonal recovery methods have been previously reported that rely exclusively on HCDR3-hybridization [25], and thus circumvent the challenge of NGS-based V_{H}-V_{L} chain pairing. (FIG. 1C).

### Example 2. FBC-Seq Applied Pilot Experiment for ROR1

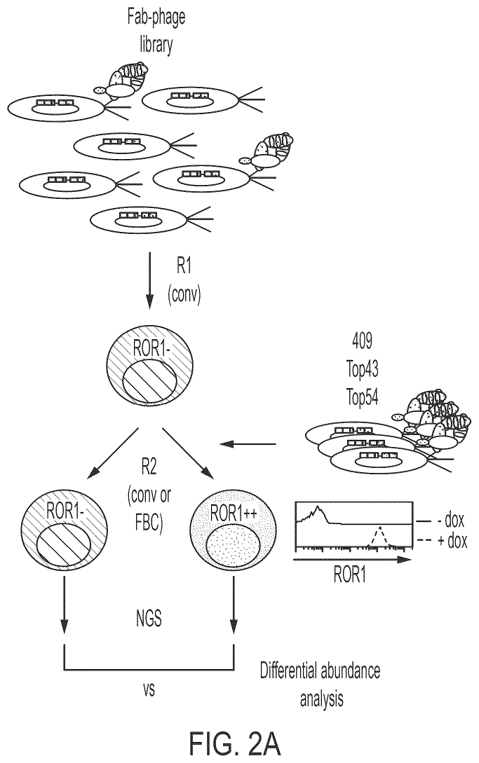

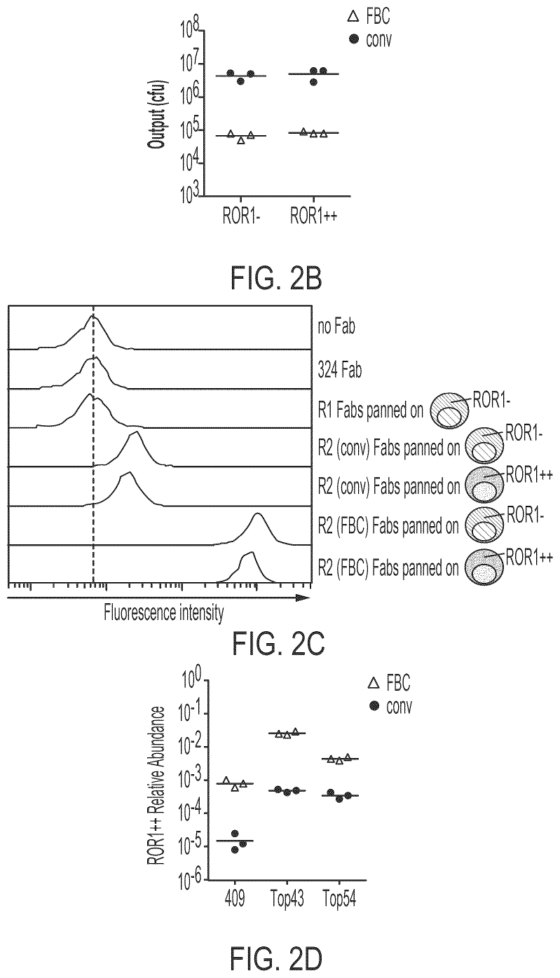

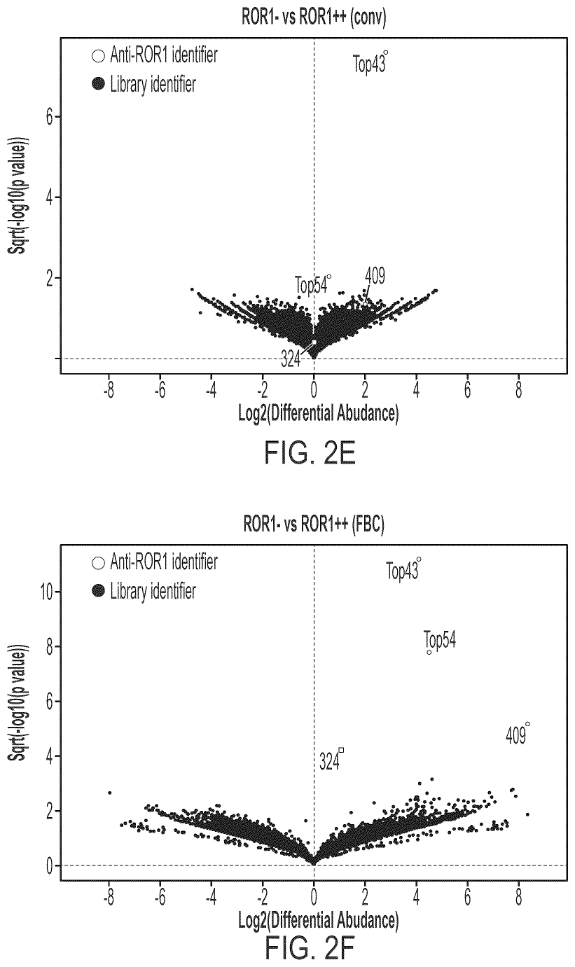

To evaluate FBC-seq, a pilot experiment was conducted using HEK293 cells with tetracycline-inducible ROR1 expression (HEK293-ROR1-TetOn). In this system, ROR1, a receptor tyrosine kinase overexpressed in hematologic and solid malignancies, served as proof-of-concept cell surface antigen. A naïve chimeric rabbit/human Fab library in phagemid pC3Csort [16] was first selected against HEK293-ROR1-TetOn cells lacking ROR1 expression due to the absence of the tetracycline derivative doxycycline (tet-off, ROR1−), thus enriching non-ROR1 cell surface binders (FIG. 2A). For the second selection round, three reporter anti-ROR1 Fab-phage clones [26] were spiked into the reamplified Fab-phage pool output of the first round at a dilution factor of 1×10⁻⁶, which approximates typical clonal representation in the second round. The three reporters had ROR1 binding affinities typical of Fab selected from naïve antibody repertoires (K_{d}=1-300 nM) [26]. The phage mixture containing ROR1 reporters was then selected against either tet-off, ROR1− HEK293 cells, or tet-on, ROR1++ HEK293-ROR1-TetOn cells. This experimental setup allowed for a controlled assessment of the ability of FBC-seq to prioritize mAbs that recognize differentially expressed epitopes. Conventional panning was conducted in parallel to FBC-seq to compare the two selection methods. After the second round, FBC output titers were ˜100-fold lower in comparison to conventional output titers, consistent with successful depletion of bald phage (FIG. 2B). Additionally, FBC panning-derived polyclonal Fabs had ˜50-fold stronger staining of ROR1− cells than polyclonal Fabs from conventional panning (FIG. 2C). Further, the NGS abundances of ROR1 reporters were ˜10-100 fold higher in the FBC compared to the conventional panning outputs. Collectively, these findings illustrate that the FBC method enhances binder enrichment via bald phage depletion (FIG. 2D). After differential abundance analysis of FBC-seq NGS outputs, ROR1 reporters were clearly separated from the pool of non-ROR1 binders. In comparison, separation was suboptimal for the conventional panning outputs (FIGS. 2D and 2F). Only the high affinity ROR1 reporter Top43 revealed clear separation in both FBC and conventional panning. These results illustrate that this phage selection and sequencing approach enables the identification of mAbs recognizing differentially expressed cell surface epitopes between two distinct whole cell substrates.

### Example 3. FBC-Seq Applied to Malignant Versus Healthy Cells

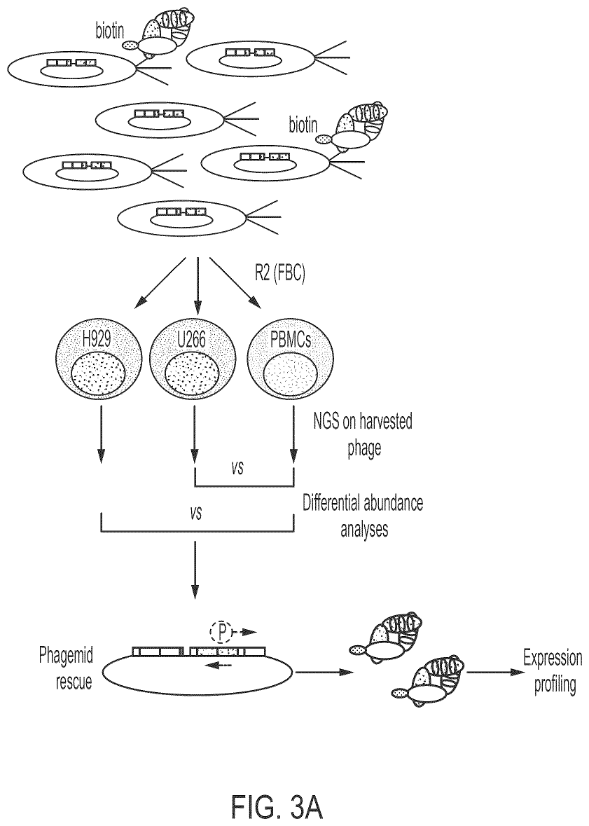

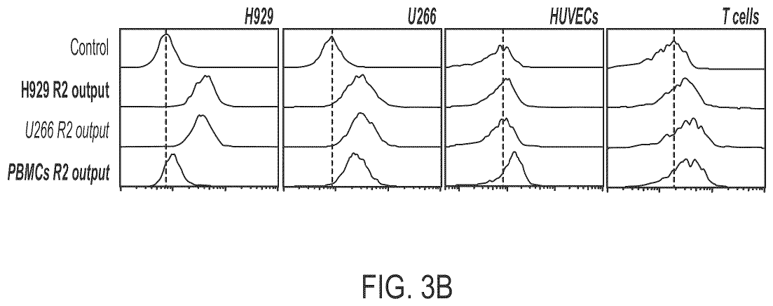

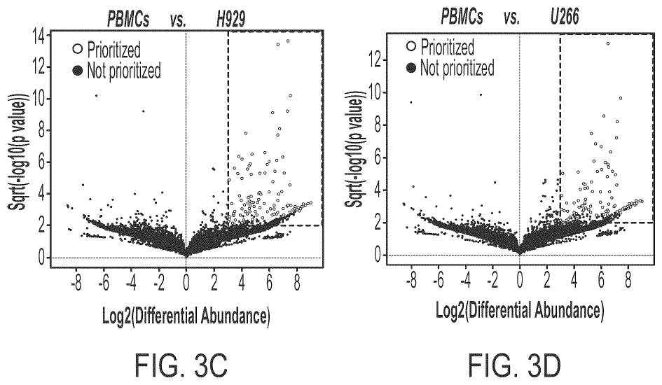

Having demonstrated the proof-of-concept that FBC-seq can select reporter Fab-phage particles at low abundance (10⁻⁶) from phage libraries, this methodology to select mAbs specific for multiple myeloma (MM) cells (FIG. 3A). A workflow was devised to enable antibody-driven target identification and prioritization (FIG. 3A). For this experiment, H929 and U266 cell lines were elected as panning targets, as obtaining enough primary MM cells for panning is unfeasible due to their low in vivo abundance and poor ex vivo survival. These cell lines share multiple characteristics with primary MM cells, including sensitivity to proteasome inhibitors (76) and expression of therapeutically or diagnostically relevant cell surface markers including CD38, CD56, CD138, and SLAMF7. (77, 78). For the differential panning, healthy donor (HD)-derived PBMCs were selected as the non-target population. PBMCs are composed of T cells (˜50%), monocytes (25%), and other critical cellular components of the innate and adaptive immune system. Since MM cells are known to aberrantly express receptors found on both lymphoid (including T, B. and NK cells) and myeloid lineages [27], we reasoned that de-prioritizing mAbs that bind healthy donor-derived peripheral blood mononuclear cells (PBMCs) should bias the selection towards more specific (i.e. MM-associated) cell surface antigens. Thus, the Fab-phage library was selected against MM cell lines H929 and U266, and in parallel against PBMCs for differential comparison. As expected, the polyclonal chimeric rabbit/human Fab pool from FBC panning outputs bound target MM cell lines, with weaker binding to T cells and endothelial cells (FIG. 3B). After NGS and bioinformatic analysis, HCDR3 identifiers were subset based on differential enrichment in the MM cell line outputs (FIGS. 3C and 3D. This yielded 151 non-redundant HCDR3 identifiers, which are hypothesized to bind antigens expressed on MM cells but not healthy PBMCs. The abundance of the prioritized HCDR3 identifiers was evenly distributed in the range of 10-2,600 by rank order. Thus, only ˜5% of the most abundant HCDR3 identifiers were prioritized, which highlights the lack of concordance between clonal abundance and predicted differential specificity within the selected polyclonal Fab pool. HCDR3 clustering analysis using CD-HIT [28] with an identity threshold of 75% yielded 141 unique clusters, suggesting that shared homology is minimal among the prioritized HCDR3 identifiers.

### Example 4. Recovery of Phagemids with Specific HCDR3 Identifiers

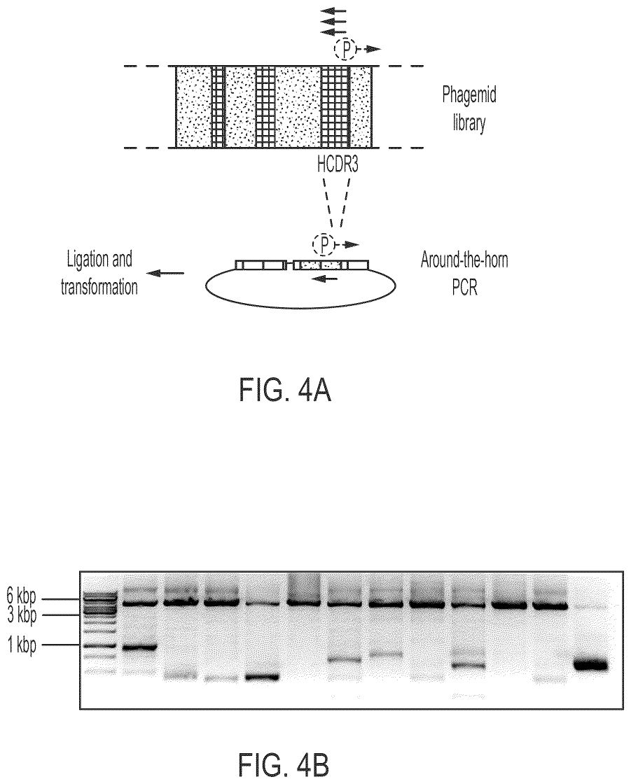

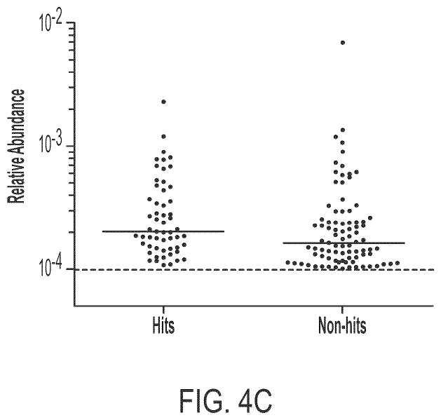

To recover Fabs of interest from pooled phagemid outputs, an around-the-horn PCR approach was then implemented, similar to a previously described method (FIGS. 4A and 4B) [29], followed by E. coli expression and flow cytometry screening of unpurified periplasmic Fab extracts [30]. In total, 55 (39%) unique MM-binding mAbs with the correct HCDR3 sequence were recovered. There was not a clear relationship between HCDR3 identifier abundance and successful hit recovery (FIG. 4C). The majority of hit mAb HCDR3 identifiers (53/55) had NGS abundances in the range of 10⁻³-10⁻⁴, highlighting the utility of deep profiling of antibody repertoires for hit identification. Initially, unpurified periplasmic Fab extracts of the 55 antibody hits were also screened by flow cytometry to assess binding to a broader panel of cell lines. Revealing high specificity for MM cell lines, 14 mAbs were then selected for further characterization.

### Example 5 Specificity Profiling and Epitope Binning of Select MM Clones

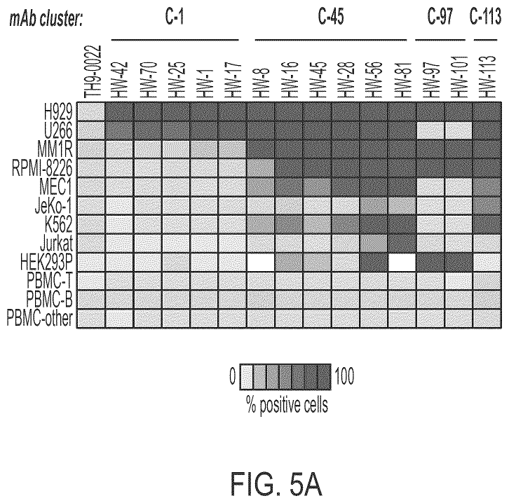

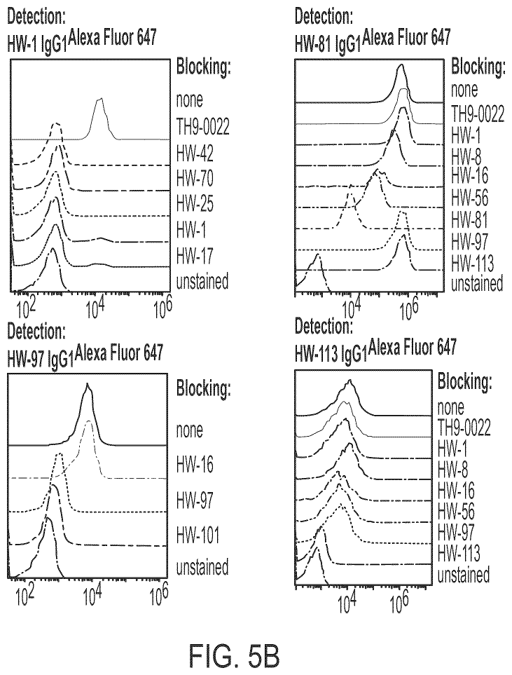

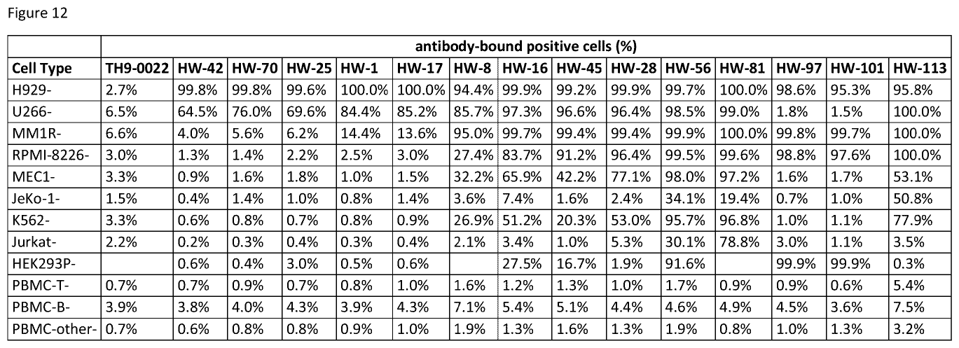

The 14 MM binders identified via FBC-seq were subsequently expressed and purified in chimeric rabbit/human IgG1 format with L234A/L235A/P329G mutations in the Fc domain to eliminate interactions with Fcγ receptors [31]. Cell reactivity profiling against a panel of cell lines then revealed four distinct reactivity patterns (mAb clusters), suggesting potential shared antigens among the panel (FIG. 5A and FIG. 12). The four clusters were named after the prototypical binders, i.e. C-1 for HW-1; C-45 for HW-45; C-97 for HW-97; and C-113 for HW-113. Importantly, none of the clusters had strong binding to healthy PBMCs, consistent with a successful in silico deselection. The clusters had distinct reactivity patterns towards leukemia and lymphoma cell lines MEC1, JeKo-1, K562, and Jurkat. To assess for common epitopes within each cluster, an epitope binning experiment was conducted. Clear competition was observed within each of the four mAb clusters, but not with mAbs in other clusters (FIG. 5B). This suggests the mAbs within each cluster share the same antigen and bind overlapping epitopes. Interestingly, the HCDR3 sequences of C-1, C-45, C-97, and C-113 do not share obvious sequence similarities (Table 2).

TABLE 2
Antibody clone characteristics

| Clone | | SEQ ID | | | SEQ ID |
|---|---|---|---|---|---|
| ID | HCDR3 | NO: | Isotype | LCDR3 | NO: |
| TH9- | ARVTYDSYGYGYAPYGMDL | 9 | kappa | LGSDSDSDIA | 10 |
| 0022 | | | | | |
| HW-42 | ARGGVDTEWYAFSS | 17 | lambda | ATSDGSGSNYQYV | 20 |
| HW-70 | AREIYLGSNF | 27 | lambda | ATADGSGSSYQFV | 30 |
| HW-25 | AREDSYYYDSFNL | 37 | lambda | ATSDGSGSSYQCV | 40 |
| HW-1 | ARDLIPTYSQDI | 47 | lambda | ATSDGSGNSYQYV | 50 |
| HW-17 | ARERYSDGRAYDI | 57 | lambda | ATDDGSGSIYQYV | 60 |
| HW-8 | VRHFRL | 67 | kappa | LGGAAAVWA | 70 |
| HW-16 | AKHWNI | 77 | kappa | LGSYSSSDTAFGG | 80 |
| HW-45 | GRRIGSARFDP | 87 | lambda | ATSDGSGSSYRLV | 90 |
| HW-28 | ARGADLHRL | 97 | lambda | ATSDGSGSSYQYV | 100 |
| HW-56 | VRGSPGYRTAISI | 107 | kappa | LGSYSSSDAS | 110 |
| HW-81 | ARGGYRNGFKL | 117 | lambda | GVIDSAVYV | 120 |
| HW-97 | ARGDVGGADNNI | 127 | lambda | ATSDGSGSSYQYV | 130 |
| HW-101 | ARDFLGVNSI | 137 | lambda | ATSDGSGSNFHFV | 140 |
| HW-113 | ARDASSPGFHGDRLDL | 147 | lambda | ATSDGIGSNYLWV | 150 |

Table 2—Antibody clone characteristics. Antibody clone characteristics. Each HCDR3 identifier is shown along with its corresponding IMGT-defined heavy- and light-chain complementarity determining region 3 (HCDR3, LCDR3) aa sequence and light chain isotype. Clones are arranged by reactivity patterns identified in FIG. 5A-B. A shared LCDR3 was seen for two clones (underlined) in distinct specificity bins, suggesting the LCDR3 is not driving antigen specificity for these clones.

### Example 6. Target Identification and Validation

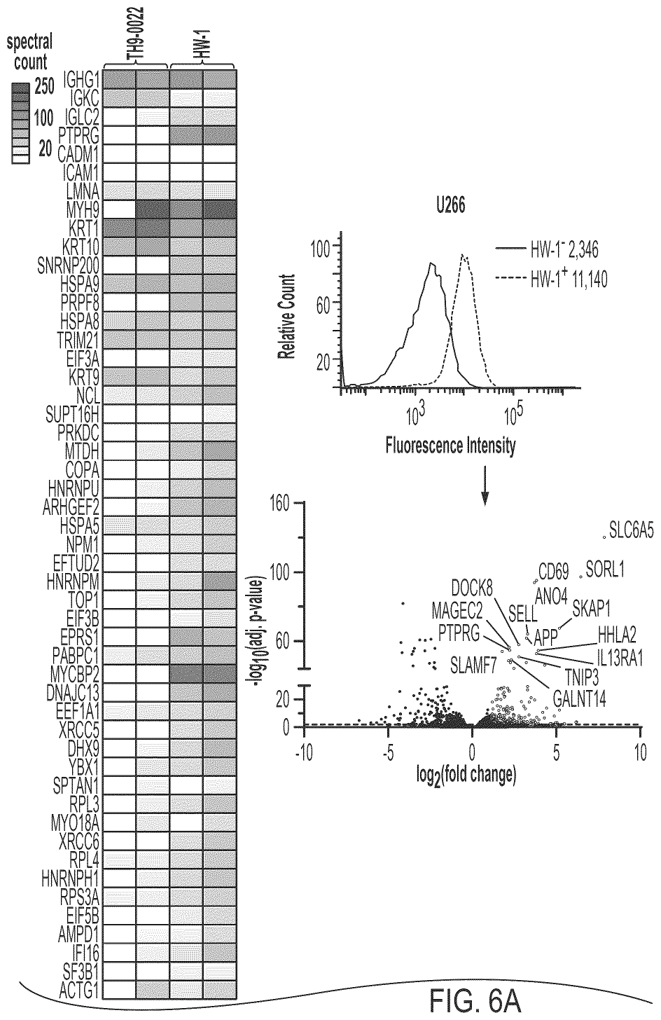

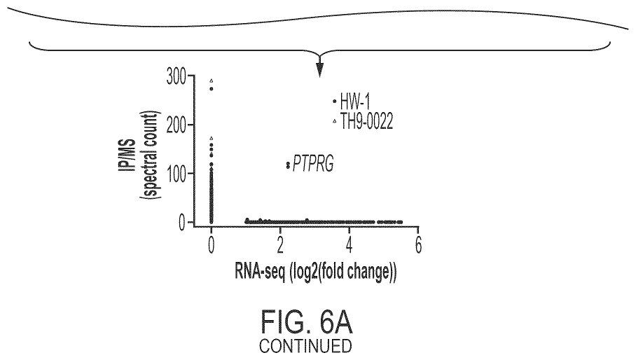

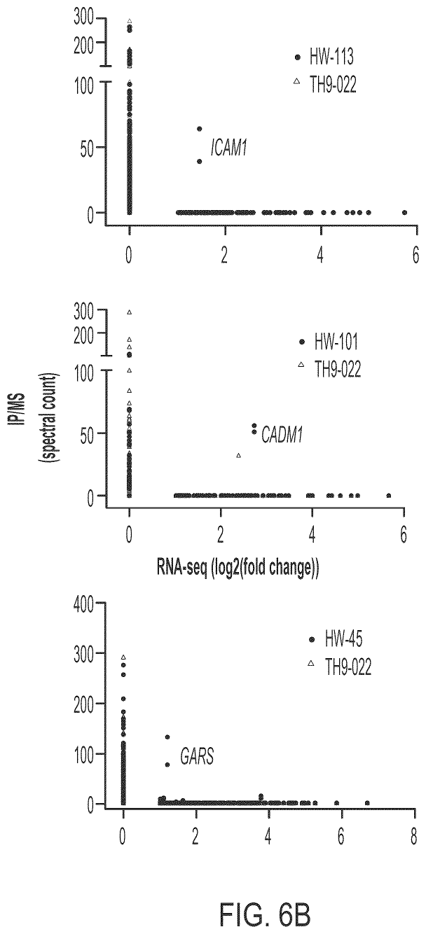

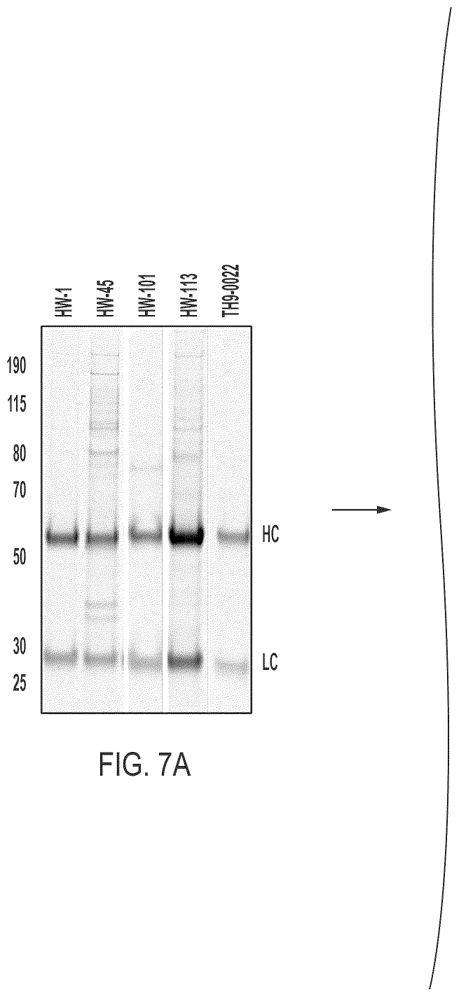

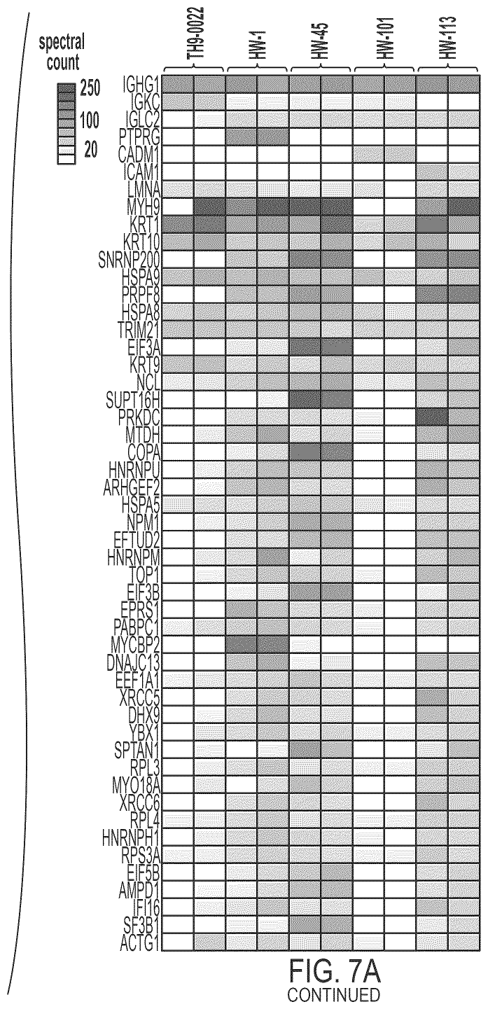

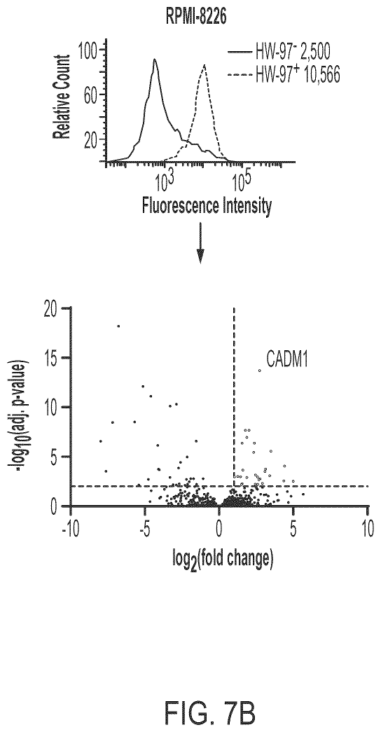

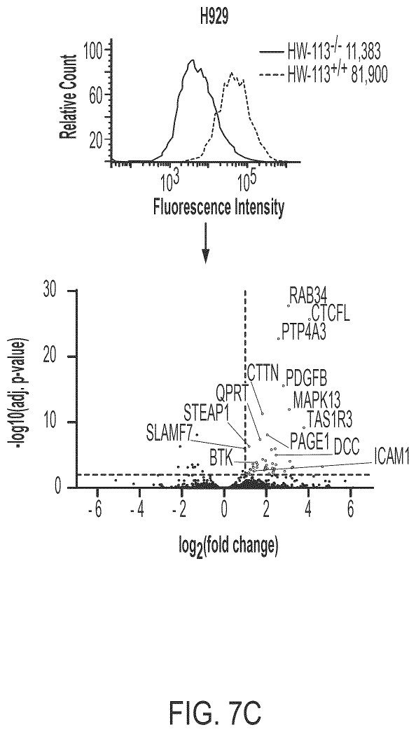

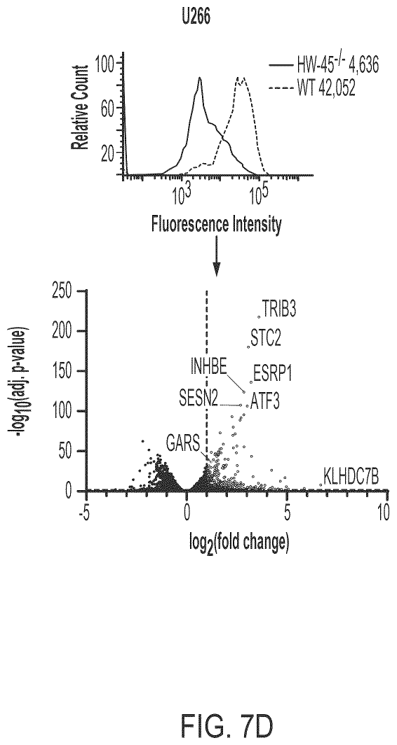

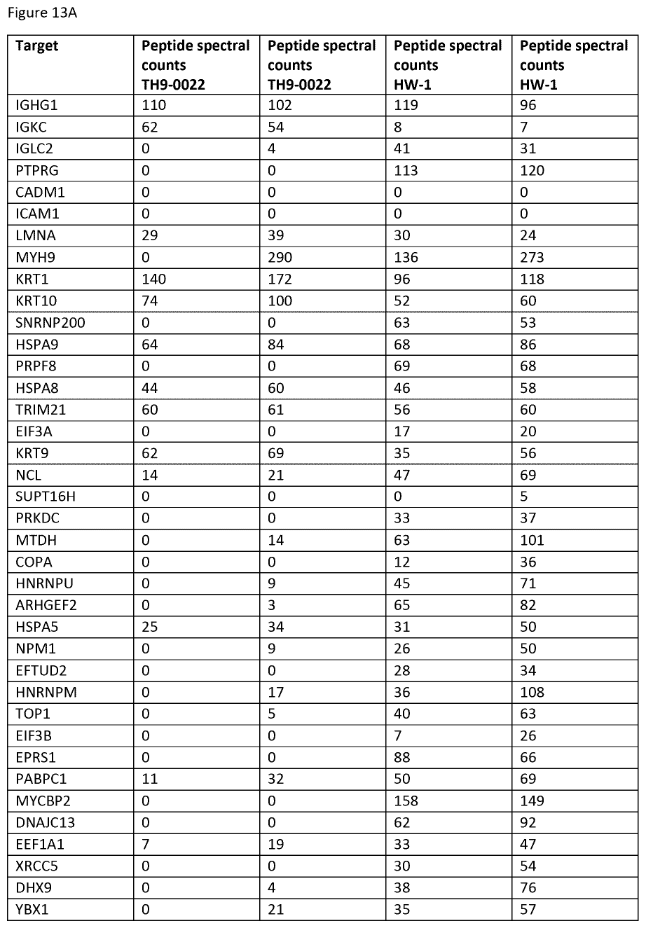

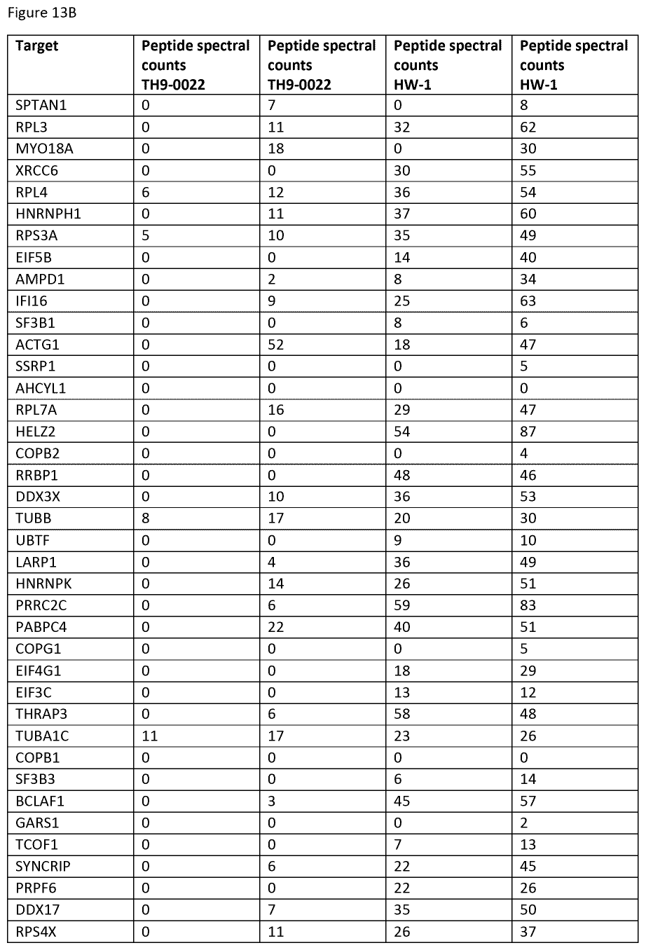

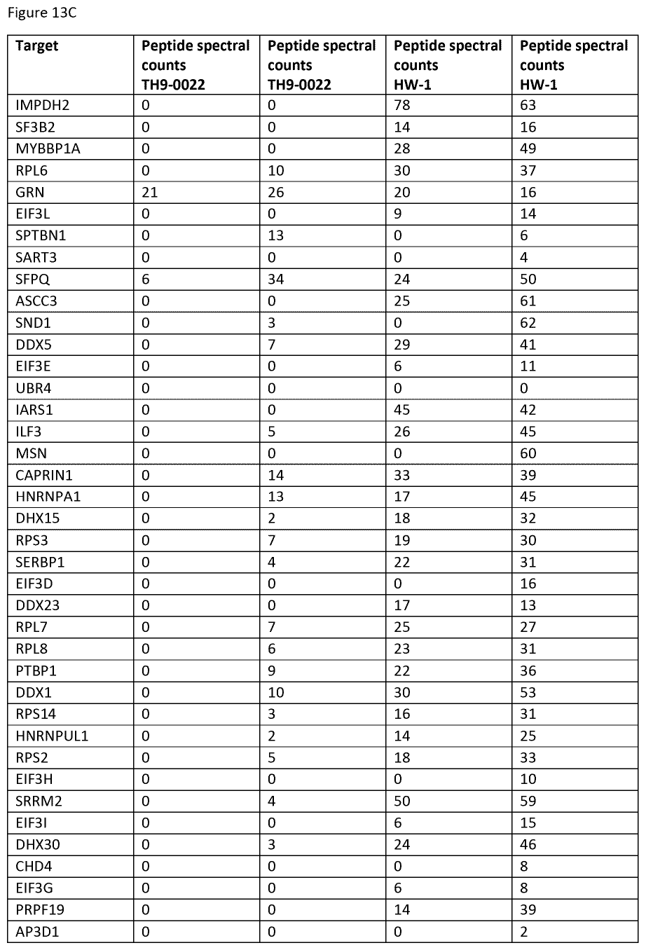

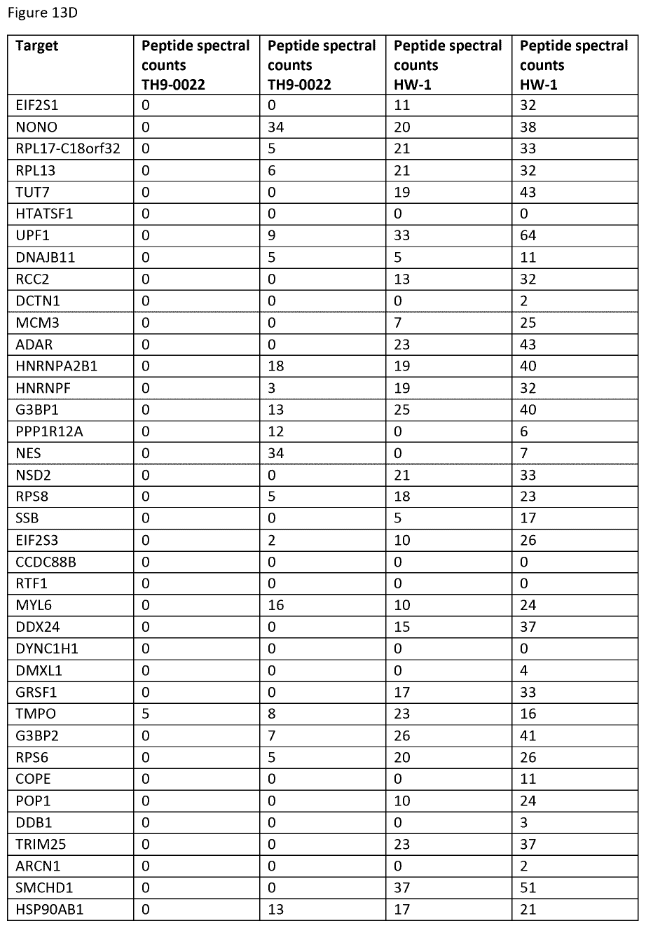

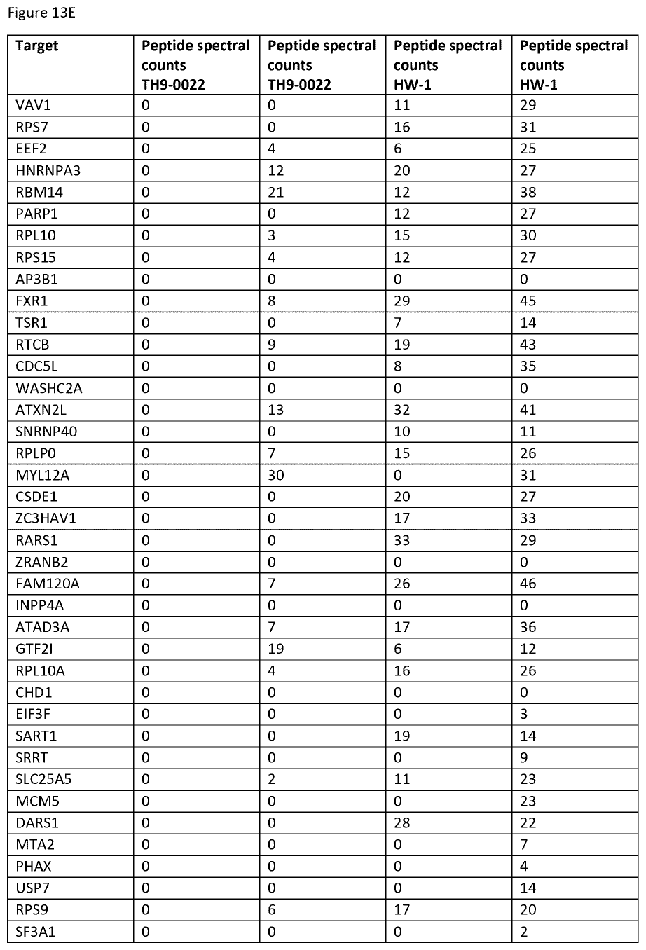

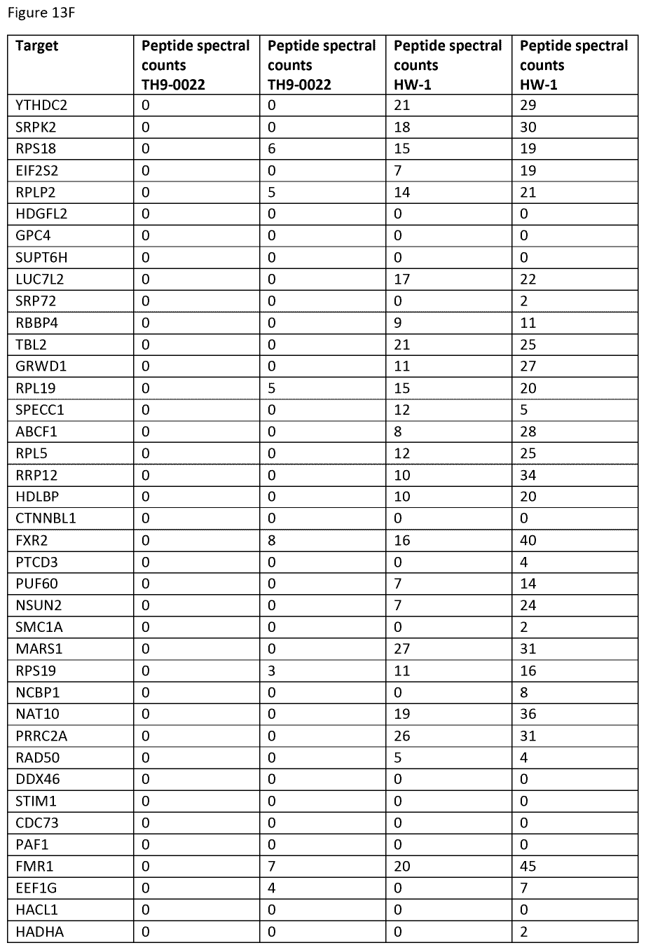

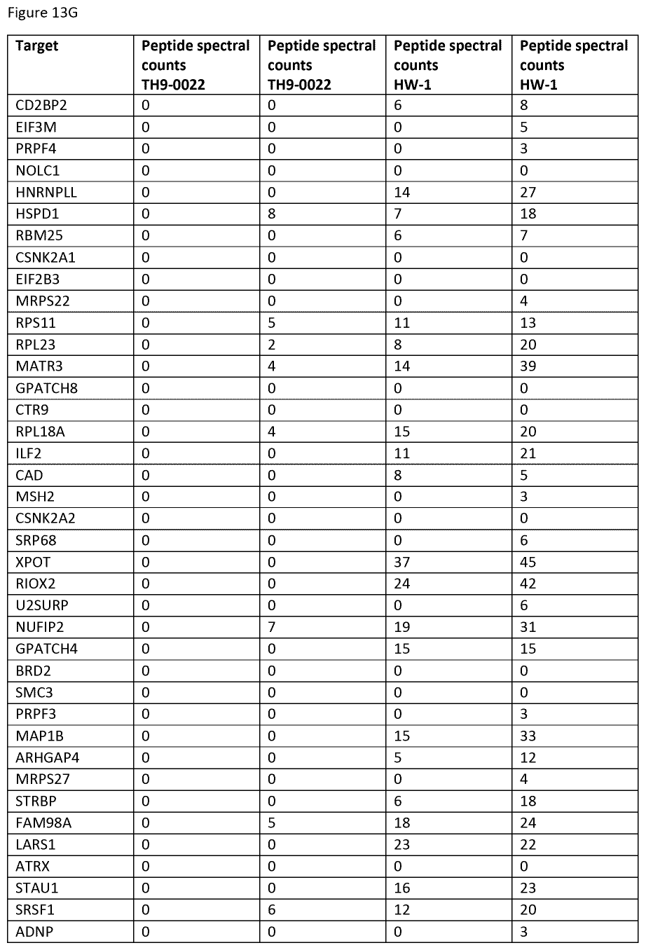

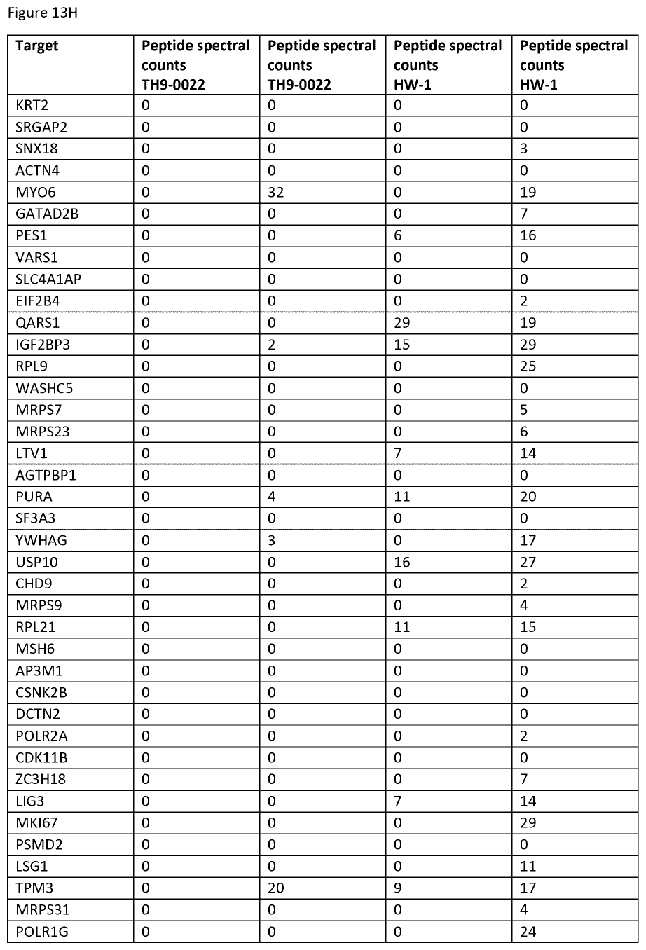

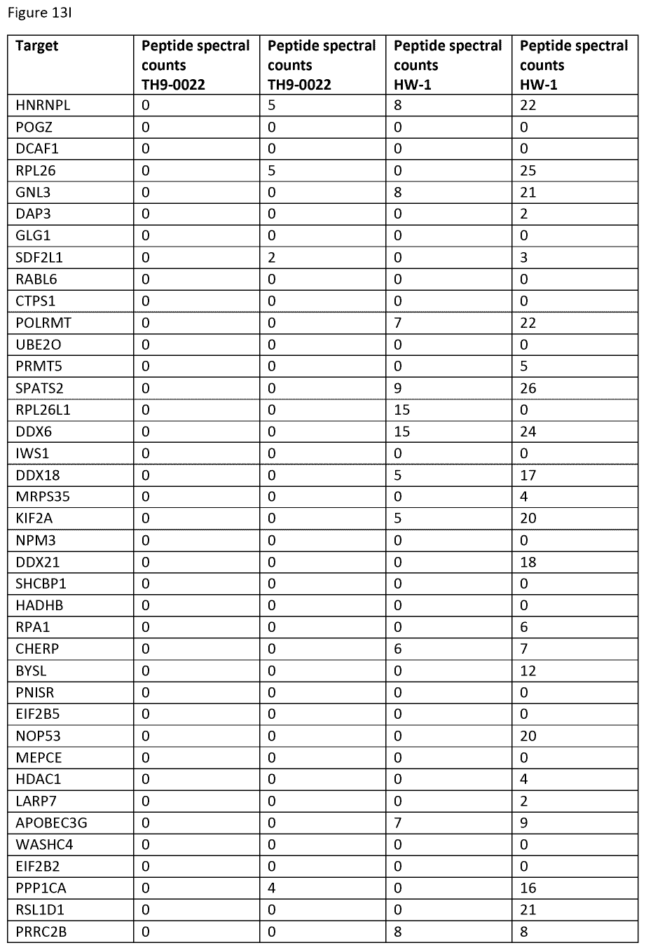

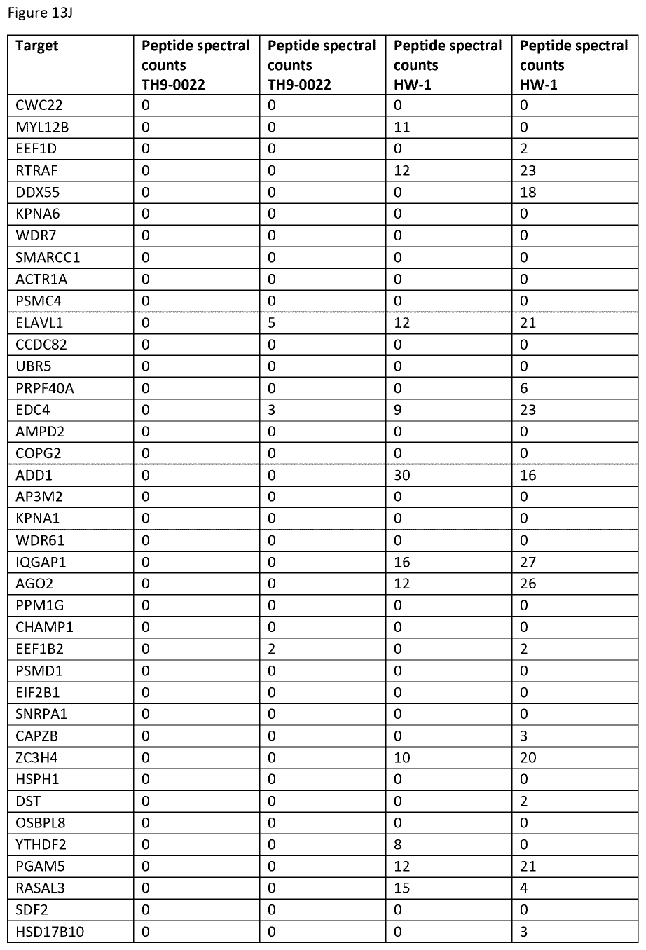

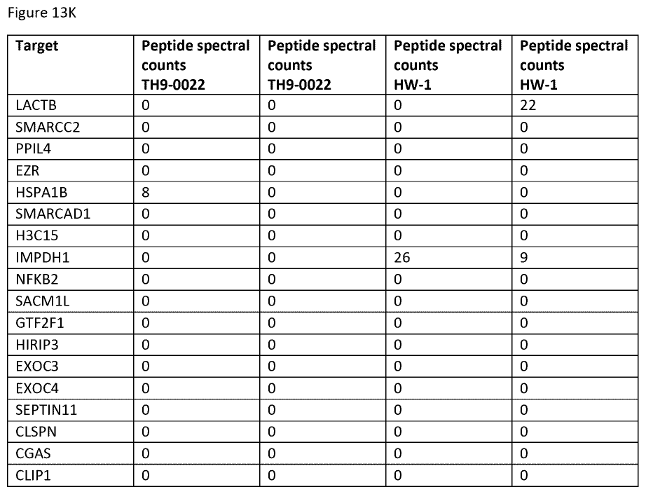

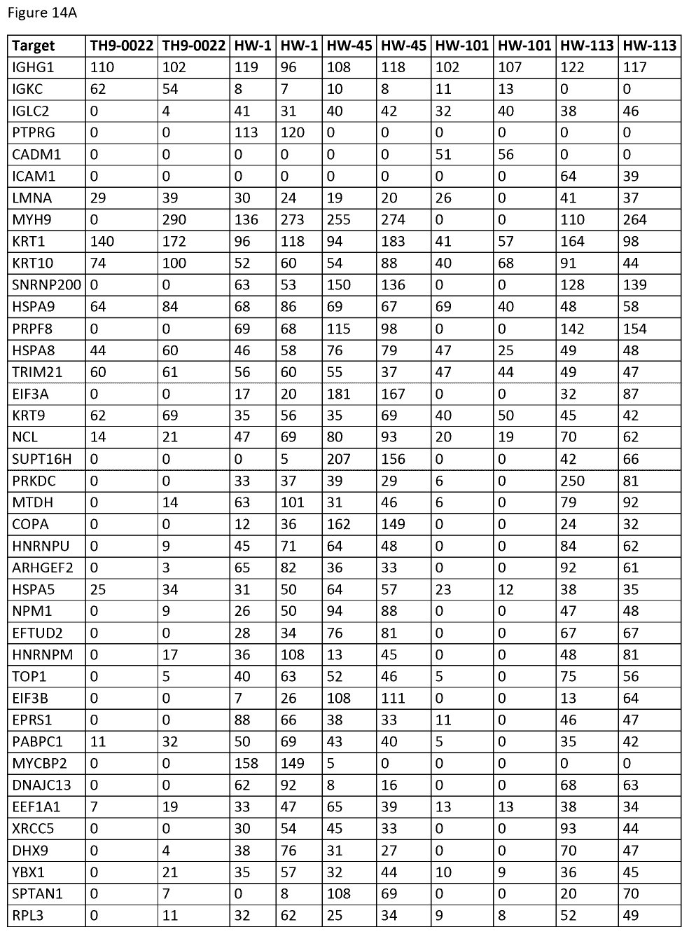

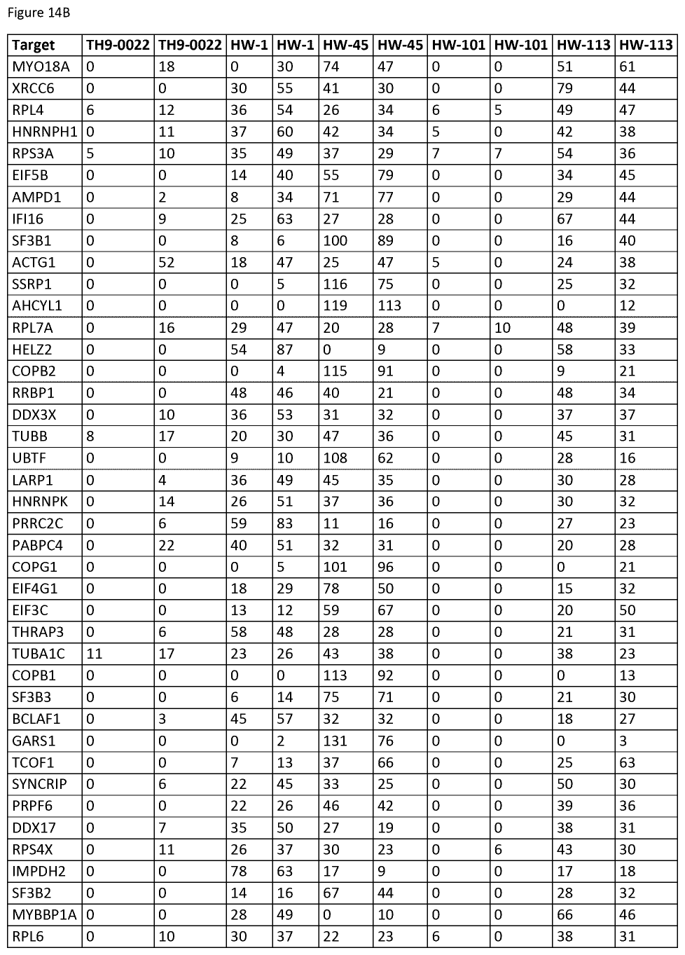

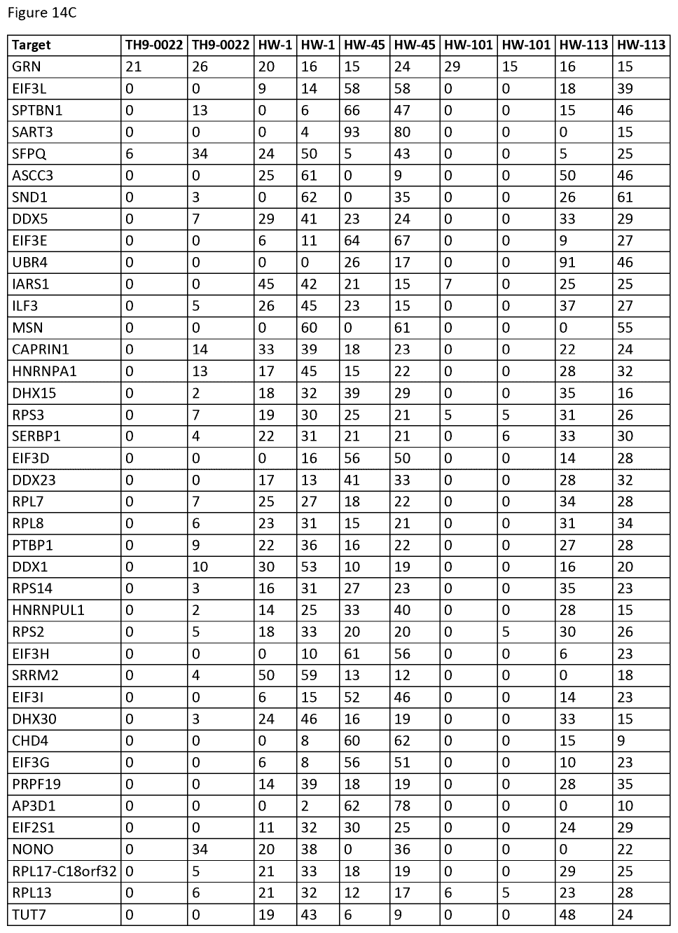

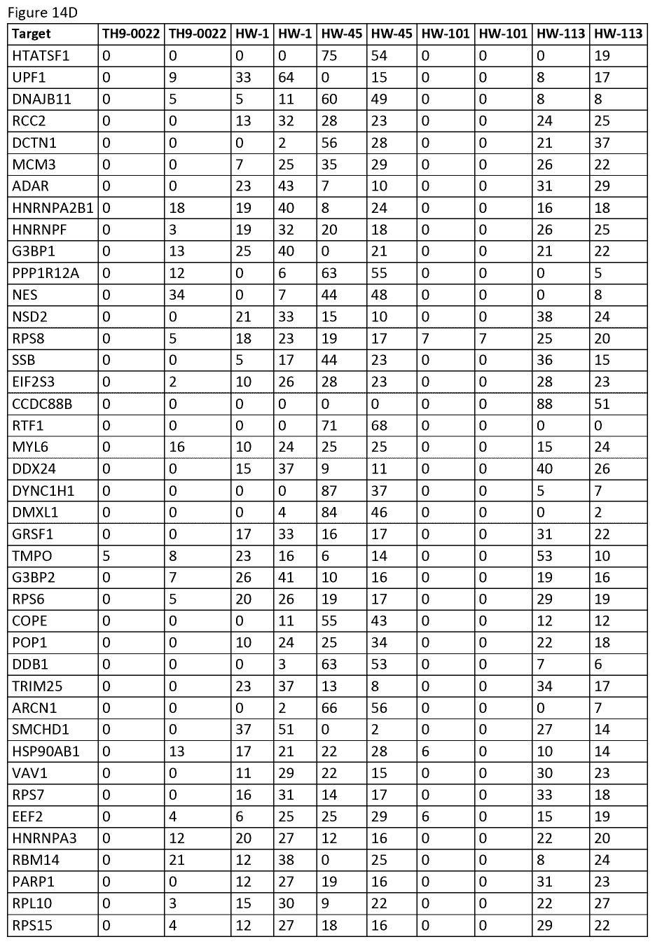

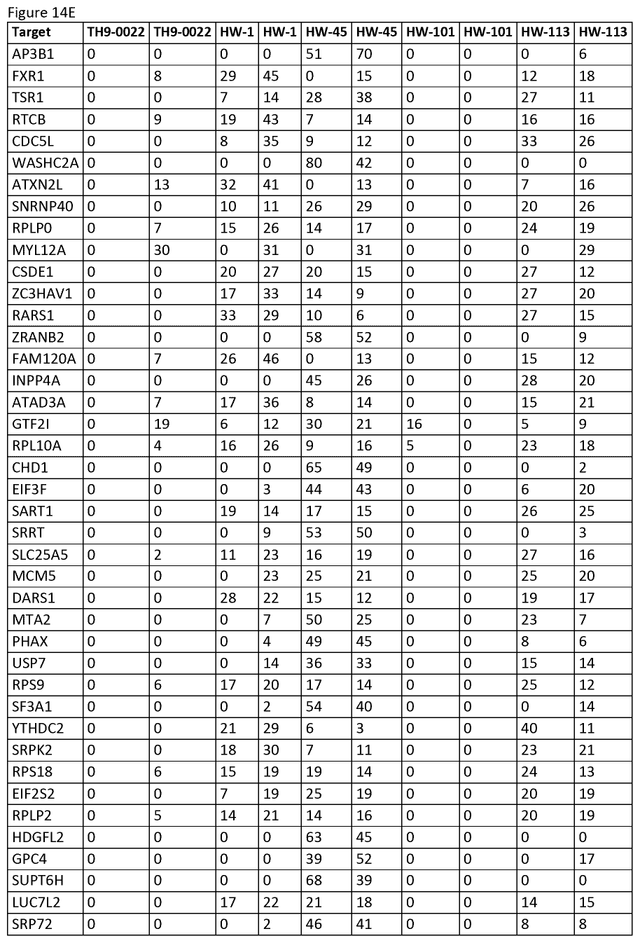

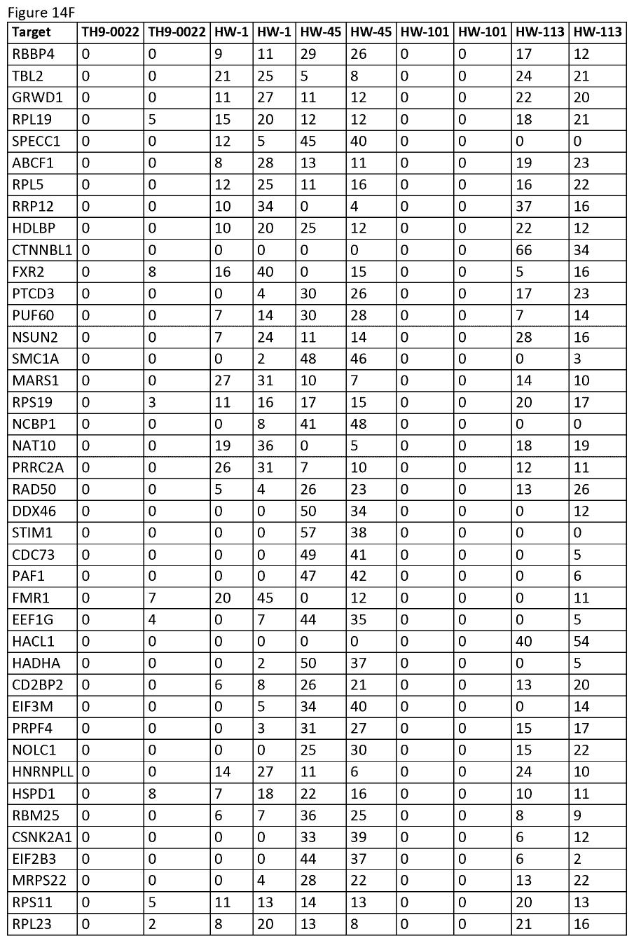

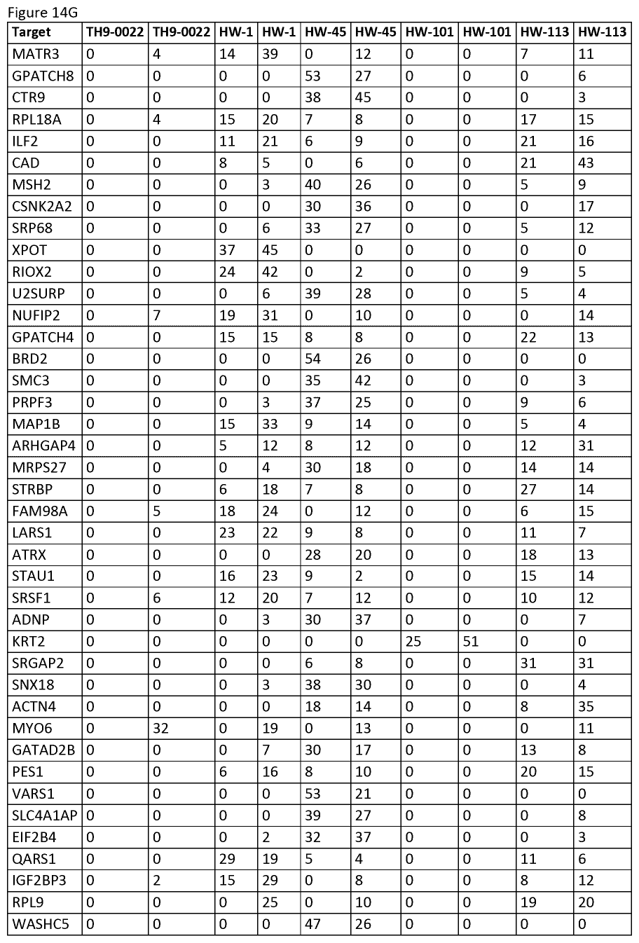

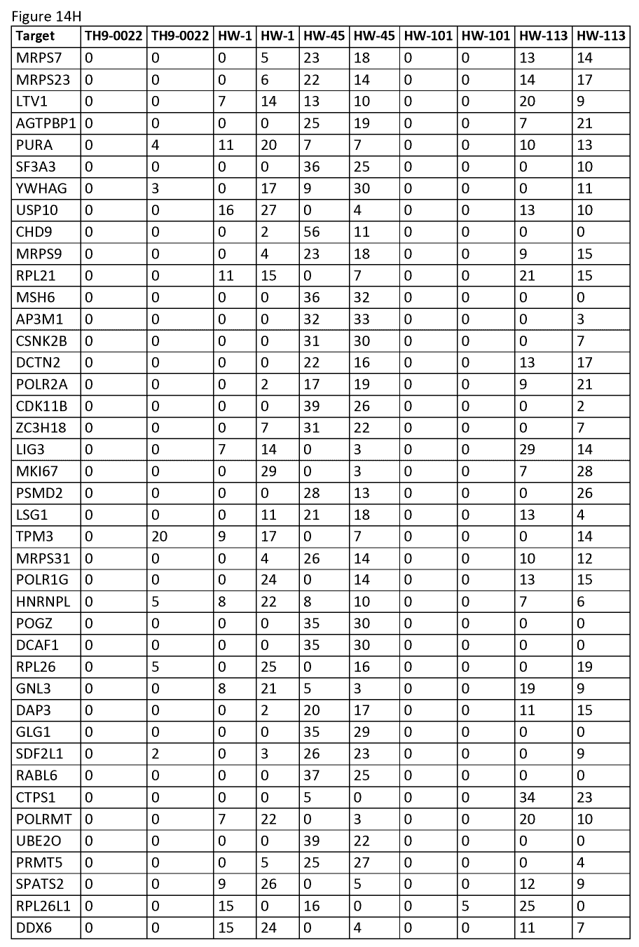

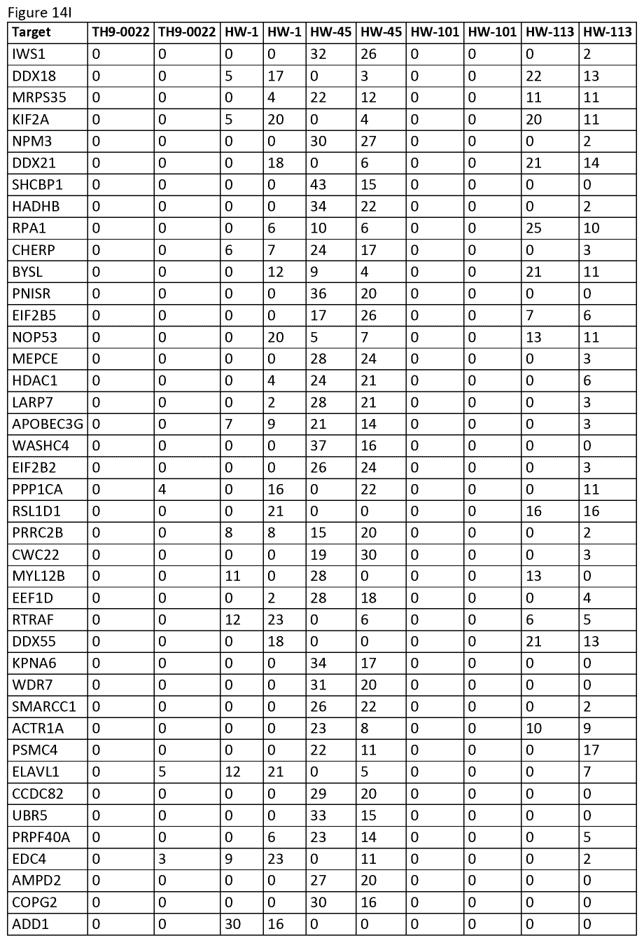

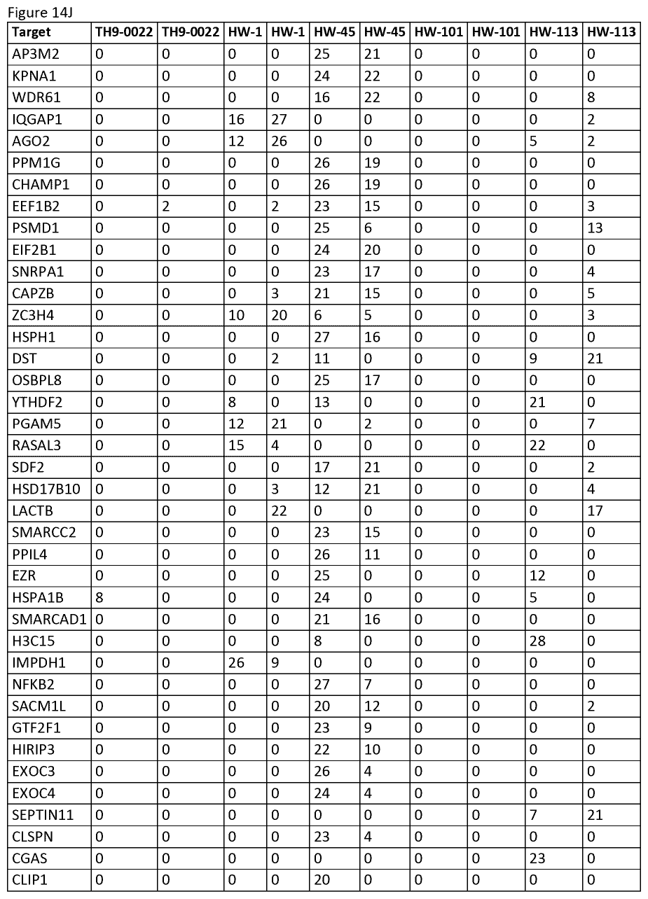

As the first step toward target identification, representative mAbs from each cluster were used to immunoprecipitate (IP) targets from H929 cell lysates. The IP elution containing chimeric rabbit/human IgG1 and prospective antigen(s) was analyzed by SDS-PAGE (FIG. 7A, left panel). Only heavy and light chain bands were observed in the pulldowns with TH9-0022 (negative control) and HW-1, a single unique band was present for HW-101, and multiple bands were present for both HW-45 and HW-113. The band pulled down by HW-101 was excised and analyzed by proteomics, but no protein specific for HW-101 and not TH9-0022 was identified. An alternative approach of analyzing the entire elution by liquid chromatography tandem mass spectrometry (LC-MS/MS) was employed and identified hundreds of proteins for each pulldown, an unwieldy number for direct biochemical validation (FIG. 6A left panel, FIG. 13A-K, FIGS. 7A and 7A Continued, FIG. 14A-J). These experiments highlight the difficulty of applying the IP/MS approach to multiple targets which may have different solubility, expression levels, and affinity for their respective antibodies. To supplement the proteomic data, we pursued an alternative transcriptomic-based methodology that is similar to a previously described target deconvolution approach [32]. Cell lines with heterogenous orphan mAb staining were selected and sorted into target-high and target-low sublines via fluorescence-activated cell sorting (FACS) (FIG. 6A, top right panel, FIG. 6A, bottom right panel, and FIG. 7B-7D). For example, U266 cells were sorted for low and high expression of the HW-1 target, followed by multiple passages, and this resulted in two cell sublines with 5-fold differential in mean fluorescence intensity (MFI) (FIG. 6A, top right panel). Transcriptomic analysis of the U266 HW-1^{-/low} and U266 HW-1^{+/high} cell sublines revealed over 200 genes with more than 2-fold differential expression and an adjusted p-value <0.01 (FIG. 6A, bottom right panel). One such gene, SLAMF7, was suggestive of a MM phenotype in the HW-1^{hi} cells. Several cell surface proteins including CD96, SELL (L-Selectin), and HHLA2 (B7-H7) appeared to be good candidate targets, yet there were also cytoplasmic genes involved in PTM processing such as GALNT14 (Polypeptide N-acetylgalactosaminyltransferase 14) that indicated the possibility of a non-protein target. The FACS/RNA-seq methodology was also successfully applied for HW-97 on RPMI-8226, and in that case one target stood out above the rest, CADM1 (FIG. 7B). For HW-113, repeat sorting of the negative and positive populations of H929 cells was needed to arrive at ˜8-fold stable differential target expression of the target, and SLAMF7 was again identified, suggesting this target is also involved in an MM phenotype (FIG. 7C). Another interesting hit was the cytoplasmic Bruton's tyrosine kinase (BTK), which is involved in B cell and plasma cell development, yet BTK inhibitors are only approved for leukemia and lymphoma, not MM (79) Lastly, negative sorting of U266 resulted in a cell subline with depleted HW-45 target expression, but positive sorting was unsuccessful at generating a stable over-expression cell line. Nonetheless, differential RNA-seq analysis was performed on HW-45^{-/-} U266 versus wild-type (unsorted) cells, which had nearly 10-fold differential antibody staining (FIG. 7D). Several hundred genes were identified as differentially expressed, yet few had readily apparent relevance.

To identify the most likely target candidates from these large orthogonal transcriptomics and proteomics data sets, the correlation between the differential expression (log₂[fold change]) of statistically significant genes identified in the FACS/RNA-seq experiment and the spectral counts of the IP/MS data for each mAb was assessed. Remarkably, for each of the four mAbs only one target was identified as being specifically pulled down (relative to the isotype control) and differentially expressed at the RNA level, providing strong evidence of successful target identification (FIG. 6A, Continued, FIG. 6B). The prospective targets were PTPRG (phosphatase receptor type γ) for HW-1, GARS (Glycyl-tRNA synthetase, GlyRS) for HW-45, CADM1 for HW-97/101, and ICAM1 (CD54) for HW-113 (Table 3).

TABLE 3
Summary of Targets and Methods

| | | | | | FACS/ | | RNA | |
|---|---|---|---|---|---|---|---|---|
| Putative | | | PBMC surface | Retrogenix | RNA- | | vs | |
| Target | HW-IgG | Localization | expression | protein array | seq | IP/MS | Protein | Validation |
| ICAM-1/CD54 | 113 | membrane | low | − | − | − | + | + |
| CADM1/IGSF4A | 97, 101 | membrane | low | + | + | + | + | + |
| PTPRG | 42, 70, 25, | membrane | no | − | − | + | + | + |
| | 1,17 | | | | | | | |
| GARS | 45, 16, 28, | cytosol | no | NA | − | − | + | + |
| | 56, 81 (8) | | | | | | | |

Table 3—Summary of targets and methods. Putative targets are listed along with the antibodies that bind to each. Note that GARS is not annotated as being on the cell surface according to the Human Protein Atlas (World Wide Web .proteinatlas.org/ENSG00000106105-GARS1). For the Retrogenix analysis mAbs in the HW-45 cluster had prohibitively high background staining against the HEK293 cells and could not be screened in the platform, NA=not applicable. The HW-8 clone competitively blocks the binding of HW-81 on the cell surface but HW-8 binding to GARS was not detected in ELISA, perhaps due to low affinity.

### Example 7. ELISA Studies

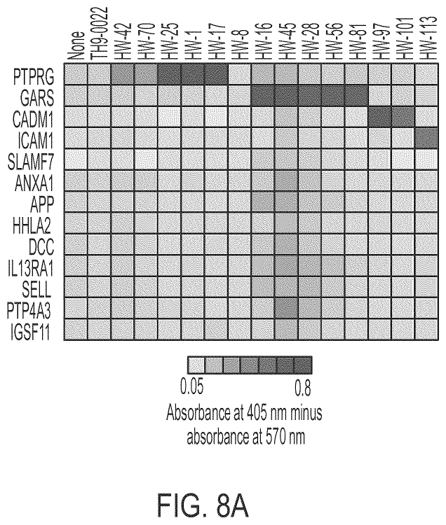

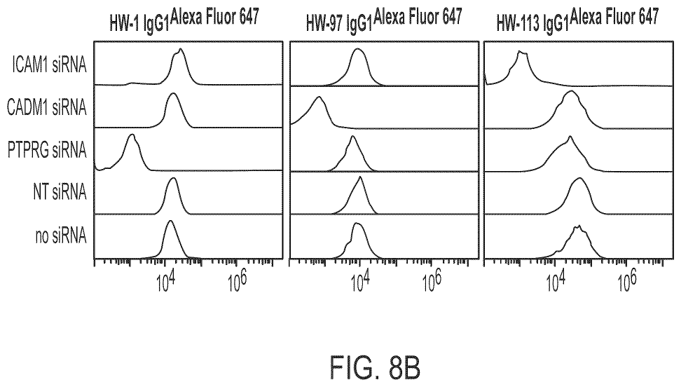

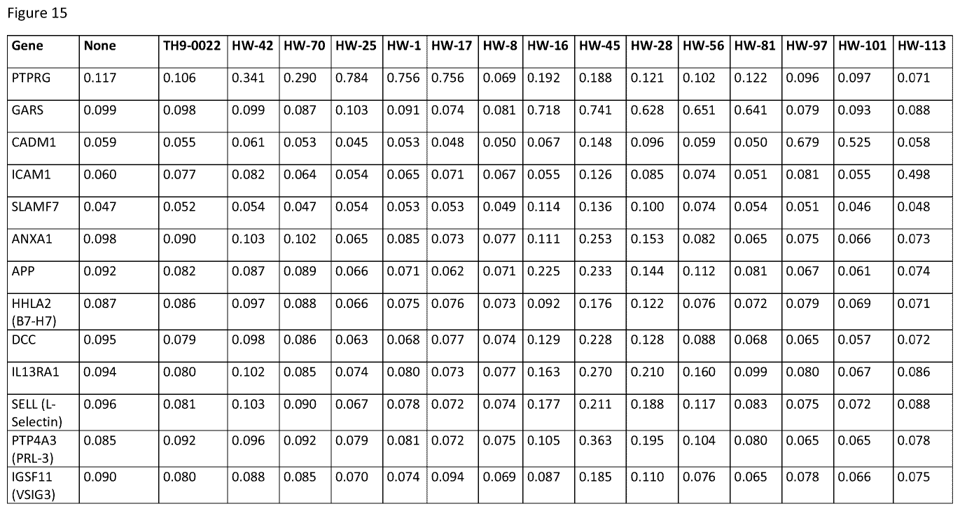

Next, to validate antibody/antigen pairs, all antibodies were examined for putative target binding via protein ELISA. This experiment provided initial confirmation of successful target deconvolution, as the mAbs from each reactivity cluster bound specifically to the predicted target from the proteomic/transcriptomic overlay (FIG. 8A and FIG. 15). For the C-45 cluster, 5 of the 6 antibodies bound strongly to GARS in ELISA, yet HW-8 did not bind. The lack of HW-8 binding in ELISA could be indicative of HW-8 having lower affinity than the other clones or binding to an alternate conformational epitope that is not available on the recombinant protein. The cell staining competition data suggested that HW-8 binds weaker than HW-81, as it was only able to partially block HW-81 staining (FIG. 8B). However, antibodies in this cluster revealed a high background in the ELISA (FIG. 8A and FIG. 15), suggesting polyreactivity or binding to a shared epitope.

### Example 8 siRNA Antibody Panel Validation

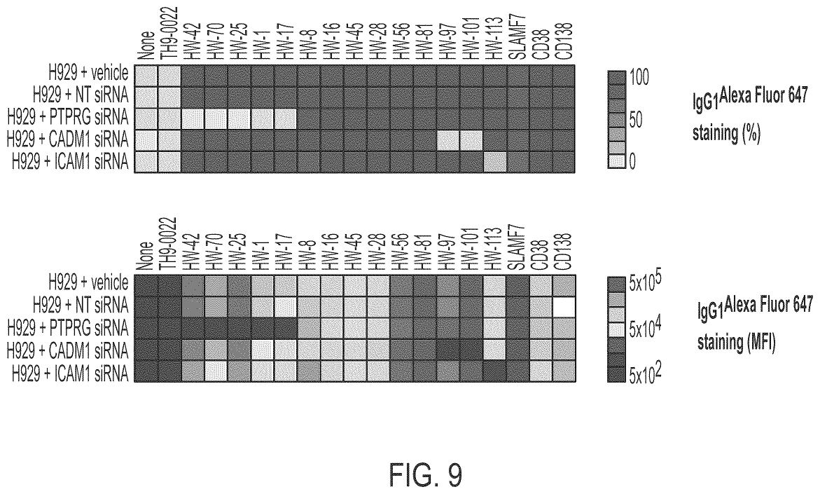

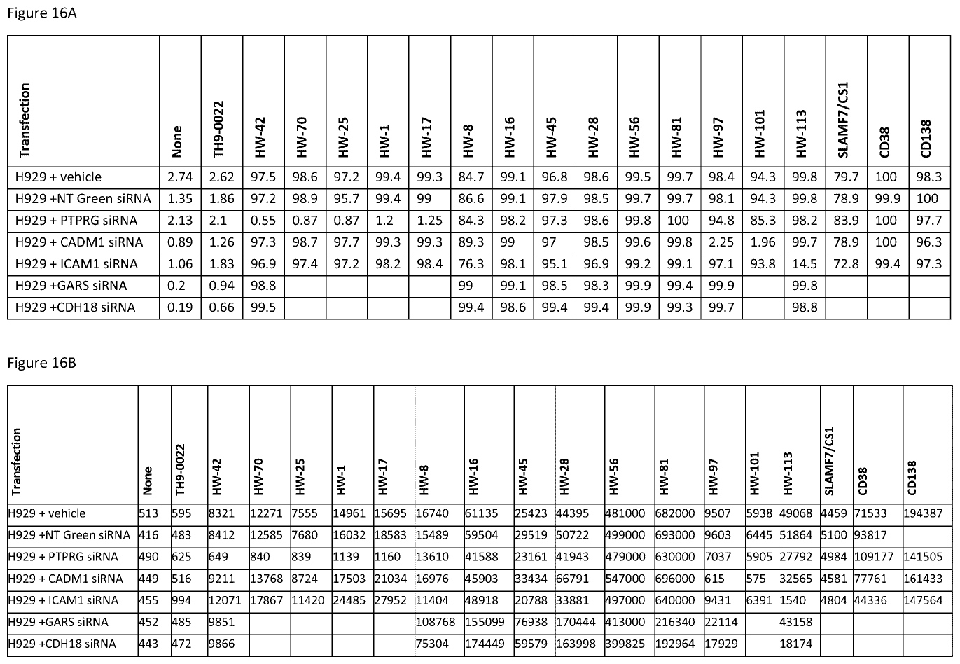

PTPRG, ICAM1, and CADM1 were then further confirmed via siRNA knockdown followed by cognate mAb staining (FIG. 8B, FIG. 9, FIG. 16A, FIG. 16B). In each case, siRNA knockdown of the putative target in H929 cells abolished cognate mAb staining. Additionally, the expression of common plasma cell and MM cell surface proteins was unaffected by the siRNA knockdowns.

### Example 9: Surface Plasmon Resonance Analysis

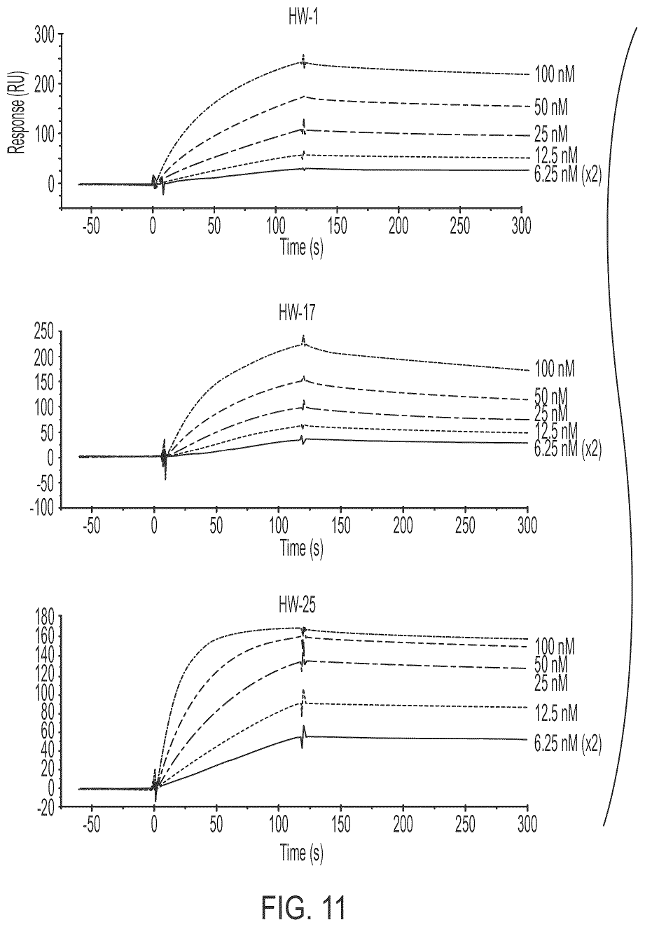

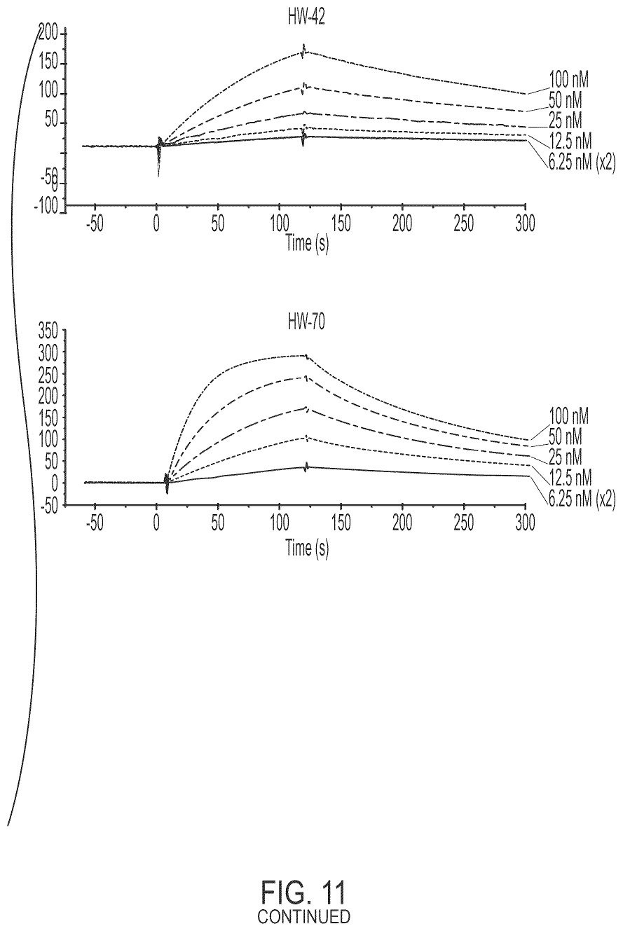

To further assess the largest cluster of deorphanized mAbs, the C-1 cluster was expressed and purified in monovalent Fab format, then evaluated via surface plasmon resonance (SPR) analysis. The antibodies in this cluster had a range of affinities for PTRPG, with K_{d} values from 0.65 nM (HW-25) to 28.9 nM (HW-42) (FIG. 11 and Table 4). These data show that the FBC-seq approach is well-suited for identifying both high- and low-affinity binders and provides multiple starting points for therapeutic antibody development campaigns.

TABLE 4
SPR analysis of anti-PTPRG Fabs

| | kₒₙ | k_{off} | |
|---|---|---|---|
| Fab | (M⁻¹s⁻¹ × 10⁵) | (s⁻¹ × 10⁻⁴) | K_{d} (nM) |
| HW-25 | 5.66 (0.20) | 3.67 (0.25) | 0.65 (0.02) |
| HW-1 | 1.95 (0.35) | 4.82 (0.22) | 2.54 (0.58) |
| HW-17 | 1.69 (0.14) | 15.8 (0.8) | 9.34 (0.70) |
| HW-70 | 8.26 (2.04) | 153.4 (12.2) | 19.1 (3.4) |
| HW-42 | 1.49 (0.11) | 42.9 (1.9) | 28.9 (1.6) |

### Example 10. Primer Sequences

Primer sequences
IgHJ forward oligonucleotides:
IgHJ 2/4:
(SEQ ID NO: 151)
5′-PO₄-GGCCCAGGCACCCTGGTCA-3′
IgHJ 3/5:
(SEQ ID NO: 152)
5′-PO₄-GGCCAGGGCACCCTGGTCA-3′
IgHJ 6:
(SEQ ID NO: 153)
5′-PO₄-GGCCCAGGGACCCTCGTCA-3′
IgHJ variant 1/2:
(SEQ ID NO: 154)
5′-PO₄-GGCCCSGGCACCCTGGTCA-3′
IgHJ variant 3:
(SEQ ID NO: 155)
5′-PO₄-GGTCCAGGCACCCTGGTCA-3′.

HCDR3-hybridizing reverse oligonucleotides used for around-the-horn phagemid rescue followed the sequence pattern 5′-CCA(N)₁₆₋₄₅-3′, where CCA encodes the reverse complement of the conserved tryptophan (W) immediately C terminal of the HCDR3 (IMGT definition), and (N)₁₆₋₄₅ encodes the reverse complement of the HCDR3 identifier trimmed from the 3′ end such that the melting temperature of the entire primer was ˜62° C. according to the Wallace rule (61).

NGS library preparation Oligonucleotides:
P5-ch1:
(SEQ ID NO: 156)
5′ACGACGCTCTTCCGATCTGCCCTTGGTGGAGGC-3′
P7-PELB:
(SEQ ID NO: 157)
5′CGTGTGCTCTTCCGATCTCAACCAGCCATGGCC-3′
P5-universal:
(SEQ ID NO: 158)
5′AATGATACGGCGACCACCGAGATCTACACTCTTTCCCTACACGACGCT
CTTCCGATCT-3′
P-Index-i: for i = 1-12.
P-Index-1
SEQ ID NO: 159
5′CAAGCAGAAGACGGCATACGAGATCGTGATGTGACTGGAGTTCAGACG
TGTGCTCTTCCGATCT-3′
P-Index-2:
SEQ ID NO: 160
5′CAAGCAGAAGACGGCATACGAGATACATCGGTGACTGGAGTTCAGACG
TGTGCTCTTCCGATCT-3′
P-Index-3
SEQ ID NO: 161
5′CAAGCAGAAGACGGCATACGAGATGCCTAAGTGACTGGAGTTCAGACG
TGTGCTCTTCCGATCT-3′
P-Index-4:
SEQ ID NO: 162
5′CAAGCAGAAGACGGCATACGAGATTGGTCAGTGACTGGAGTTCAGACG
TGTGCTCTTCCGATCT-3′
P-Index-5:
SEQ ID NO: 163
5′CAAGCAGAAGACGGCATACGAGATCACTGTGTGACTGGAGTTCAGACG
TGTGCTCTTCCGATCT-3′
P-Index-6:
SEQ ID NO: 164
5′CAAGCAGAAGACGGCATACGAGATATTGGCGTGACTGGAGTTCAGACG
TGTGCTCTTCCGATCT-3′
P-Index-7:
SEQ ID NO: 165
5′CAAGCAGAAGACGGCATACGAGATGATCTGGTGACTGGAGTTCAGACG
TGTGCTCTTCCGATCT-3′
P-Index-8:
SEQ ID NO: 166
5′CAAGCAGAAGACGGCATACGAGATTCAAGTGTGACTGGAGTTCAGACG
TGTGCTCTTCCGATCT-3′
P-Index-9:
SEQ ID NO: 167
5′CAAGCAGAAGACGGCATACGAGATCTGATCGTGACTGGAGTTCAGACG
TGTGCTCTTCCGATCT-3′
P-Index-10:
SEQ ID NO: 168
5′CAAGCAGAAGACGGCATACGAGATAAGCTAGTGACTGGAGTTCAGACG
TGTGCTCTTCCGATCT-3′
P-Index-11:
SEQ ID NO: 169
5′CAAGCAGAAGACGGCATACGAGATGTAGCCGTGACTGGAGTTCAGACG
TGTGCTCTTCCGATCT-3′
P-Index-12:
SEQ ID NO: 170
5′CAAGCAGAAGACGGCATACGAGATTACAAGGTGACTGGAGTTCAGACG
TGTGCTCTTCCGATCT-3′

Primer index combinations were selected to maintain proper color balancing.

### Example 11: Summary of Antibody Sequences

Table 5 provides a summary of SEQ ID NOs for antibody heavy chain variable region, light chain variable region, IMGT CDRs, heavy chain, and light chain sequences for clones.

TABLE 5
Summary of SEQ ID NOs for Clones

| Clone | | Heavy | | | | | Light | | | |
|---|---|---|---|---|---|---|---|---|---|---|
| ID | VH | chain | HCDR1 | HCDR2 | HCDR3 | VL | chain | LCDR2 | LCDR3 | LCDR3 |
| TH9-0022 | 5 | 7 | — | — | 9 | 6 | 8 | — | — | 10 |
| HW-42 | 11 | 13 | 15 | 16 | 17 | 12 | 14 | 18 | 19 | 20 |
| HW-70 | 21 | 23 | 25 | 26 | 27 | 22 | 24 | 28 | 29 | 30 |
| HW-25 | 31 | 33 | 35 | 36 | 37 | 32 | 34 | 38 | 39 | 40 |
| HW-1 | 41 | 43 | 45 | 46 | 47 | 42 | 44 | 48 | 49 | 50 |
| HW-17 | 51 | 53 | 55 | 56 | 57 | 52 | 54 | 58 | 59 | 60 |
| HW-8 | 61 | 63 | 65 | 66 | 67 | 62 | 64 | 68 | 69 | 70 |
| HW-16 | 71 | 73 | 75 | 76 | 77 | 72 | 74 | 78 | 79 | 80 |
| HW-45 | 81 | 83 | 85 | 86 | 87 | 82 | 84 | 88 | 89 | 90 |
| HW-28 | 91 | 93 | 95 | 96 | 97 | 92 | 94 | 98 | 99 | 100 |
| HW-56 | 101 | 103 | 105 | 106 | 107 | 102 | 104 | 108 | 109 | 110 |
| HW-81 | 111 | 113 | 115 | 116 | 117 | 112 | 114 | 118 | 119 | 120 |
| HW-97 | 121 | 123 | 125 | 126 | 127 | 122 | 124 | 128 | 129 | 130 |
| HW-101 | 131 | 133 | 135 | 136 | 137 | 132 | 134 | 138 | 139 | 140 |
| HW-113 | 141 | 143 | 145 | 146 | 147 | 142 | 144 | 148 | 149 | 150 |

### Example 12. Materials and Methods

### Cell Lines and Primary Cells

Variants of HEK1293 cell lines with tetracycline-inducible ROR1 expression (tet-off, ROR1−; tet-on, ROR1++) were generated from the HEK293 Tet-On 3G cell line (Takara). Cells were cultured at 37° C. in 500 CO₂ in complete medium consisting of Dulbecco's Modified Eagle's Medium (DMEM) (Thermo Fisher Scientific) containing 10% (v/v) fetal bovine serum (FBS; Atlas Biologicals) and 1× penicillin-streptomycin (PS) (Thermo Fisher Scientific). Cells were grown to ˜50% confluence in a 6-well dish using complete medium. Immediately prior to transfection, the medium was gently changed to 2 mL of complete medium lacking FBS. In parallel, 2 μg of pTRE3G-ROR1-IRES-Puro was mixed with 0.25 mL Opti-MEM Reduced Serum Medium (Thermo Fisher Scientific), and then 2 μg polyethylenimine (Polysciences) was added and mixed. After letting the mixture sit at room temperature (RT) for 15 min, it was added to the sub-confluent HEK293 Tet-On 3G cells. After 37° C. incubation for 5 h, the transfection mixture was removed. The cells were recovered for 2 days in complete medium supplemented with 1 μg/mL doxycycline (Takara) and then harvested and resuspended in complete medium supplemented with 1 μg/mL doxycycline and 0.5 ng/mL puromycin (MilliporeSigma). After 4 days of puromycin selection, the cells were re-plated in 96-well tissue culture plates in limiting dilution with complete medium lacking puromycin and colonies were expanded. To screen for successful transfectants and inducible expression, each clone was grown in complete medium in the presence or absence of the tetracycline derivative doxycycline (1 μg/mL). After 2 days of incubation, cells were harvested and analyzed for ROR1 expression by flow cytometry using anti-ROR1 Fab 324 (10 μg/mL) and PE-conjugated goat anti-human IgG, F(ab′)₂ (Jackson ImmunoResearch #109-116-097, 1:100 dilution).

The following cell lines were all cultured at 37° C. in 5% CO₂ in complete medium consisting of RPMI-1640 medium containing 10% (v/v) FBS (BioFluid) and 1×PS: NCI-H929 (H929), U266, MM.1R, RPMI-8226, MEC1, JeKo-1, K562, and Jurkat. HEK293P cells were cultured identically, but with DMEM as the base medium. Fresh healthy donor PBMCs were bought from AllCells and cryopreserved prior to use with 50% FBS, 10% DMSO in RPMI-1640 medium.

### Cloning

pTRE3G-ROR1-IRES-Puro. ROR1 ECD-encoding and PDGFRB transmembrane domain-encoding cDNA sequences were PCR-amplified using plasmids pCEP4-ROR1-HIS [26] and PB2.0-scFab (J.C., C.R., and H.P., manuscript submitted), respectively, as templates, joined via overlap extension PCR, and SalI/FseI-cloned into pTRE3G-IRES (Takara), yielding plasmid pTRE3G-ROR1-IRES. Using Linear Puromycin Marker (Takara) and DasherGFP plasmid (DNA2.0) as templates, cDNA sequences encoding puromycin resistance gene and T2A-DasherGFP were PCR-amplified, joined via overlap extension PCR, and MluI/NheI-cloned into pTRE3G-ROR1-IRES, yielding plasmid pTRE3G-ROR1-IRES-Puro.

Anti-ROR1 Fabs into pC3Csort. Chimeric rabbit/human anti-human ROR1 Fabs ERR1-409, ERR1-TOP43, and ERR1-TOP54 [26] respectively, referred to in shorthand as 409, Top43 and Top54, were transferred from pET11a expression vectors into pC3Csort by asymmetric SfiI cloning as previously described [16].

Recovery of Fab clones. The HCDR3 differential abundance analysis (as described in FBC-seq bioinformatics) yielded representative HCDR3 and framework region 4-encoding DNA sequences for HCDR3 amino acid identifiers. From these templates, phosphorylated IGHJ-hybridizing forward primers and HCDR3-hybridizing reverse primers were designed for each prioritized unique HCDR3 identifier such that their melting temperatures were ˜62° C. using the Wallace rule [61]. The following conditions were used for around-the-horn PCR recovery of phagemids using HCDR3 hybridizing primers: 1×Q5 Reaction Buffer, 0.2 mM dNTPs, 0.25 μM phosphorylated IGHJ forward primer, 0.25 μM HCDR3-specific reverse primer, 0.02 U/μL Q5 Hot Start High-Fidelity DNA Polymerase (New England Biolabs), and 20 pg/μL of purified phagemids from either H929 or U266 panning outputs. 25 μL reactions were carried out for each HCDR3-specific reverse primer using the following thermocycling conditions: 98° C. for 5 min, followed by 35 cycles of 98° C. for 10 s then 72° C. for 3 min, followed by a final elongation at 72° C. for 7 min, followed by cooling to 4° C. Subsequently, ˜5 kb fragments corresponding to linear phagemid amplicons were agarose gel-extracted and purified using a MinElute PCR Purification Kit (Qiagen). The PCR products were then directly subjected to self-ligation using T4 DNA ligase (Roche) overnight at 16° C. in a total volume of 2.5 μL, and 1 μL from each ligation reaction was used to transform 10 μL of chemically competent XL1-Blue cells (Agilent) according to the manufacturer's procedure. After recovery of bacteria in 100 μL SOC medium for 1 h at 37° C., 90 μL and 5 μL were plated from each transformation on separate LB agar plates containing 100 μg/mL carbenicillin and 10 μg/mL tetracycline. Plates were incubated overnight at 37° C. to facilitate colony growth. See Example 9 for all primer sequences (SEQ ID NOs:151-155). For Fab expression, the Fab-encoding cassette was PCR-amplified from the pC3Csort phagemid and cloned into the pET11a expression vector by asymmetric SfiI cloning as previously described [16].

Conversion of Fab to IgG1. The chimeric rabbit/human light chains (rbVL-huCL) were PCR amplified from pC3Csort phagemids, adding an NheI site and a signal peptide encoding sequence at the 5′ end and a NotI site at the 3′ end, and then cloned into mammalian expression vector pCEP4 (Thermo Fisher Scientific) via NheI/NotI. Human IgG1 Fc encoding gene fragments with L234A, L235A, P329G mutations to eliminate all Fcγ receptor interactions [31] were custom synthesized (Twist Biosciences). Chimeric rabbit/human Fd fragments (rbVH-huCH1) were amplified similarly to the light chains, except the reverse primer added a CH2 hybridization site instead of a NotI site. This PCR product was subsequently annealed to Fc fragments having a 3′ NotI site via overlap extension PCR and cloned into pCEP4 via NheI/NotI.

### Panning

FBC selection against HEK293-ROR1-TetOn cells. A first round of panning against HEK293-ROR1-TetOn cells was carried out in a conventional fashion (no FBC) as previously described [16] against 3×10⁷ HEK293-ROR1-TetOn cells, grown in absence of doxycycline, in a volume of 600 μL selection buffer containing 3% (w/v) bovine serum albumin (BSA), 0.025% (w/v) NaN₃, and 1 mM EDTA in PBS (pH 7.4). A second round of panning was conducted with or without the FBC method implemented. For this panning round, ROR1-binding Fab-phage clones 409, Top43, and Top54 were reamplified separately. Supernatants from each of these monoclonal preparations were spiked into the pC3Csort round 2 input phage preparation (400 mL reamplification culture) prior to the first phage precipitation at a dilution factor of 1×10⁻⁶. Input phage preparation, including Fab-phage biotinylation, proceeded as previously described [16]. In brief, ˜10¹¹ cfu/μL phage in Tris-buffered saline (TBS) were subjected to Sortase A-mediated biotinylation by incubation with 10 μM Sortase A, 250 μM custom synthesized Gly3-biotin (LifeTein), and 10 mM CaCl₂ for 15 min at 37° C. Following standard phage precipitation with polyethylene glycol, washing with TBS, and resuspension in selection buffer, the phage preparation was then split into nine equal parts of 500 μL of selection buffer. Each part was used as an input for panning in triplicate on 7×10⁶ HEK293-ROR1-TetOn cells treated with either 0 or 100 ng/mL doxycycline in a total selection volume of 200 μL (FIG. 2A). Phagemid-bearing output bacteria (ER2738 E. coli) were harvested as previously described and stored as glycerol stocks [16].

Differential selection against U266 and H929 cells. A first round of panning was conducted using a conventional (non-FBC) approach against H929 and U266 cells. The general procedure for this is described above with minor exceptions: 25×10⁶ cells were used as targets for this first round of panning in a total selection volume of 500 μL. The phagemid-bearing output bacteria from these separate panning experiments were pooled and used to reamplify phage for the second round of panning. In this round 2, FBC panning was carried out as described [16] and as briefly summarized above. For this, 25×10⁶ cells were panned in triplicate against H929 cells, U266 cells, and fresh healthy PBMCs (9 panning replicates in total).

### NGS Library Production

For each panning replicate, 0.5 mL of the bacterial panning output glycerol stocks was subjected to phagemid preparation using the QIAprep Spin Miniprep Kit (Qiagen). Rabbit VH domains were then amplified using the following PCR conditions: 1×Q5 Reaction Buffer, 0.2 mM dNTPs, 0.2 μM P5-ch1 primer, 0.2 μM P7-PELB primer, 0.02 U/μL Q5 Hot Start High-Fidelity DNA Polymerase (New England Biolabs), and 2 ng/μL pC3Csort library template. 100 μL (4×25 μL) reactions were carried out for each panning replicate using the following cycling conditions: 98° C. for 5 min, followed by 4 cycles of 98° C. for 10 s, 60° C. for 20 s, and 72° C. for 30 s, followed by 8 cycles of 98° C. for 10 s, 72° C. for 30 s, followed by a final elongation at 72° C. for 7 min. followed by cooling to 4° C. Amplified rabbit V_{H} domains (˜450 bp) were then gel purified using a MinElute PCR Purification Kit (Qiagen). These products were used as templates for a second PCR reaction in which the P5/P7 cluster forming sites and adaptors were appended. Reaction conditions were the same as above for the first PCR reaction, except using antisense P5-universal primer and barcoded P7-Index-i sense primers at 0.2 μM. Unique P7 indices were used for each replicate. The template concentration for this second PCR was 0.2 ng/μL. PCR products (˜500 bp) were isolated by gel purification as above, and then further purified using Agencourt AMPure XP beads (Beckman Coulter) at a 1:1 volumetric ratio per the manufacturer's protocol. Products were eluted in 25 μL TE buffer, pooled, and analyzed on a MiSeq sequencer (Illumina) using MiSeq Reagent Kit v3 (150-cycle) in a single end configuration with reads originating from the P5 primer binding site. See Example 9 for all primer sequences (SEQ ID NOs:156-170).

### Bioinformatic Analysis

NGS library analysis. R version 3.5.1 (32-bit) was used for the analysis. For each of the comparisons (ROR1− vs. ROR1+ FBC, ROR1− vs. ROR1+ Conv), the R script “Differential phage antibody abundance v5” was run. The dependencies of this script include the following R packages: Biostrings, dada2, seqinr, plyr, and DESeq2. For each comparison, the parameter library_names was modified to match the de-multiplexed FASTQ files corresponding to the appropriate panning replicates. The parameters targ_name and non_targ_name were also modified appropriately for the different comparisons. The dispersion shrinkage functionality of DESeq2, critical for the algorithm's statistical inference of differential abundance, was well fit for both FBC and conventional analyses. The length distribution and amino acid composition of the HCDR3 identifiers were consistent with previously published deep sequencing analyses of rabbit antibody repertoires (26, 85) indicating this bioinformatic approach faithfully extracts rabbit HCDR3s.

### FBC-Seq Bioinformatics

Antibody bioinformatics was conducted in R. FASTQ files were first filtered for reads bearing correct hybridization primer sequences. Reads were then translated, HCDR3s extracted via a REGEX recognizing HCDR3 junctions in the rabbit IgG repertoire, and counts aggregated for each pool. In parallel, representative HCDR3 and framework region 4-encoding sequences were extracted for each unique HCDR3 identifier. An HCDR3 aa count matrix was then generated for differential enrichment analysis using DESeq2. R code has been deposited to the GitHub FBCseq repository World Wide Web (github.com/hedawils/FBCseq). Raw FASTQ files from FBC-seq outputs along with processed data files have been deposited into the NCBI Gene Expression Omnibus (GEO) database under accession number GSE222897 (World Wide Web ncbi.nlm.nih.gov/geo/query/acc.cgi?acc=GSE222897).

### Flow Cytometry

Staining protocol. Cells were harvested (5×10⁴ cells per sample), washed once with phosphate-buffered saline (PBS) at RT, and a second time with ice-cold FACS buffer (PBS, 0.5% BSA, 2 mM EDTA, 0.1% NaN₃). Cells were stained in 50 μL of primary antibody for 30 min on ice, washed twice with 100 μL FACS buffer, stained with 50 μL of diluted secondary antibody, washed two more times in FACS, and then resuspended in 50 μL FACS buffer. Samples were acquired on an Accuri C6 Plus or a BD FACSCanto II flow cytometer (both from BD Biosciences). Data were analyzed using FlowJo version 10 software. Primary antibodies: Fab (10 μg/mL), IgG1 (0.1 to 50 μg/mL), or 25 μL polyclonal Fabs from periplasmic extracts. Polyclonal Fabs were produced by diluting bacterial stocks by a factor of 100 in autoinduction media and incubating overnight to induce expression as previously described [16]. Secondary antibody: PE-conjugated goat anti-human IgG, F(ab′)₂ (Jackson ImmunoResearch #109-116-097, 1:100 dilution). Viability stains: DAPI (Cell Signaling Technology) or SYTOX AADvanced Dead Cell Stain (Thermo Fisher Scientific). Labeling: The panel of chimeric rabbit/human IgG1s was labeled using the Alexa Fluor 647 Labeling Kit (Thermo Fisher Scientific, #A20173) to facilitate single-step staining. Competition: Unlabeled IgG1s were added at saturating concentrations of 60 μg/mL to block the target receptors, incubating on ice for 30 min, followed by the addition of an equal volume of the Alexa Fluor 647 labeled antibody at 2 μg/mL, and the mixture was incubated on ice for another 30 min.

### Protein Expression and Purification

IgG1: To generate mAbs in IgG1 format with L234A/L235A/P329G mutations [31], the heavy and light chain pCEP4 plasmids were co-transfected into Expi293 cells (Thermo Fisher Scientific) at a ratio of 1:2 (HC:LC), with the total DNA concentration fixed at 1 μg/mL according to the manufacturer's recommendations. The culture supernatants were harvested 4 to 6 days post-transfection and purified via affinity chromatography using 1-mL HiTrap Protein A HP columns (Cytiva #17-0402-01) on an ÄKTA FPLC instrument (Cytiva). Fab: The PTPRG-binding Fabs in the pET11a expression vector were expressed in Rosetta E. coli (Novagen) as previously described [62], and the secreted His-tagged Fabs were purified using immobilized metal affinity chromatography (IMAC) with a cOmplete His-Tag Purification Column (MilliporeSigma). Antigens: Recombinant proteins used in ELISAs were bought commercially from R&D Systems (SELL-Fc-His, HHLA2-Fc, IL13RA1-Fc-His, IGSF11-Fc, ANXA1-His, His-PTP4A3, DCC-His, ICAM1-Fc), Acro Biosystems (CADM1-His, APP-His), or Sino Biological (GARS-His). Alternatively, the ECD was expressed and purified using Expi293 cells in-house (PTPRG-Fc, SLAMF7-StrepTag) [26]. For PTPRG-Fc, a gene fragment encoding an immunoglobulin heavy chain signal peptide (MDWTWRILFLVAAATGAHS; SEQ ID NO:172) followed by the ECD of human PTPRG (aa 20-736) was custom synthesized (Twist Bioscience) and fused to a human Fcγ1-encoding gene fragment with an N-terminal GGGS (SEQ ID NO:173) linker and SEPKSSDKTHTCPPCP (SEQ ID NO:174) hinge after custom cloning into a pTwist mammalian cell expression vector with CMV promoter (Twist Bioscience). The plasmid was transfected into Expi293 cells using the ExpiFectamine 293 Transfection Kit (Thermo Fisher Scientific). PTPRG-Fc was then purified from supernatants by Protein A affinity chromatography and confirmed by SDS-PAGE.

Immunoprecipitation. H929 cells (from fresh cultures or cell pellets) were lysed at a density of 2×10⁷ cells/mL using lysis buffer composed of 50 mM Tris-HCl pH 7.6 (MilliporeSigma), 150 mM NaCl (MilliporeSigma), 1.0% Triton X-100 (Thermo Fisher Scientific), 1 mM EDTA (Research Products International), and 1× protease inhibitor cocktail (MilliporeSigma #P2714). After 30 min of rocking at 4° C., and lysates were centrifuged at 18,400×g for 20 min at 4° C. Subsequently, 1-mL aliquots of the supernatants were pre-cleared twice with Protein G agarose (Thermo Fisher Scientific) by adding 50 μL of 50% resin slurry and incubating 30 min with end-over-end tumbling at 4° C., followed by centrifugation at 16,000×g for 5 min and pooling of the pre-cleared cell lysate aliquots. Representative mAbs from each cluster were immobilized on Protein G agarose at a ratio of 25 μg IgG1 per 30 μL of 50% slurry, diluted to 500 μL with Dulbecco's PBS (DPBS) and incubated for 1 h at 4° C. with tumbling, followed by centrifugation at 5,000×g for 30 s and 2 washes with lysis buffer. After the last wash, the ˜15 μL of IgG1-coated resin was blocked with 10 μL of 10% BSA (MilliporeSigma). Next, 1 mL of the pooled pre-cleared cell lysate was added to the IgG1-coated resin and incubated for 3 h at 4° C. with tumbling. The immune complexes were pelleted by centrifugation at 2,500×g at 4° C. for 30 s, and gently resuspended to wash for 3 min in 1 mL of lysis buffer, which was repeated once with lysis buffer and twice with an identical wash buffer lacking Triton X-100. Finally, the immune complexes were eluted from the resin with three batch-wise steps using 30 μL of elution buffer (6 or 8 M urea (MilliporeSigma), 20 mM Tris-HCl pH 7.5, and 100 mM NaCl) and incubating 15 min at RT with 450 rpm orbital agitation. Finally, 16 μL of the eluate (˜20%) was analyzed on a reducing SDS-PAGE gel stained with SYPRO Ruby dye (Bio-Rad Laboratories), while the rest was stored at −80° C. prior to LC-MS/MS analysis.

### LC-MS/MS Analysis and Protein Identification

Protein elutions in 6 or 8 M urea were reduced with 15 mM dithiothreitol at 56° C. for 30 min followed by alkylation with 30 mM iodoacetamide in the dark at RT. The elutions were then diluted to 1 M urea with 50 mM ammonium bicarbonate pH 7.8 and digested overnight at 37° C. following the addition of 4 μg trypsin (Promega). LC-MS/MS analysis of extracted peptides was subsequently carried out using an Orbitrap Fusion Tribrid mass spectrometer (Thermo Fisher Scientific) as previously described [63], following 2-μg capacity ZipTip C18 (MilliporeSigma) sample clean-up according to the manufacturer's instructions, with the exception that separation was performed on as EASY-Spray PepMap RSLC C18 column (2 μm, 100 Å, 75 μm×50 cm, Thermo Fisher Scientific), and ions were created with an EASY-Spray Source (Thermo Fisher Scientific) held at 45° C. using a voltage of 2.1 or 2.5 kV. The gradient was also modified to deliver solvent B (80/20 acetonitrile/water, 0.1% formic acid) from 5-25% in 135 min, followed by 25-44% solvent B in 45 min, 44-80% solvent B in 0.10 min, a 10 min hold of 80% solvent B, a return to 5% solvent B in 5 min, and finally with another 5-min hold of 5% solvent B. All flow rates were 250 nL/min delivered using an Easy-nLC 1000 Liquid Chromatography system (Thermo Fisher Scientific). Solvent A consisted of 0.1% formic acid. Tandem mass spectra (MS/MS) were searched against the Human Proteome FASTA database from UniProtKB (UP000005640) downloaded on Sep. 14, 2020. All MS/MS spectra were searched using Thermo Proteome Discoverer 2.5.0.400 (Thermo Fisher Scientific) considering fully tryptic peptides with up to two missed cleavage sites and precursor and fragment ion mass tolerances set to 10 ppm and 0.02 Da, respectively. Variable modifications considered during the search included methionine oxidation (15.995 Da) and asparagine and glutamine deamidation (0.984 Da). Cysteine carbamidomethylation (57.021 Da) was considered as a static modification. At the time of the search, the protein database built as described above had 74,854 entries. A database of common contaminant proteins available with Proteome Discoverer version 2.5.0.400 was also used in the search. Proteins were identified at 99% confidence with XCorr score cut-offs as determined by a reversed database search [64]. Finally, the protein and peptide identification results were visualized with Scaffold version 5.0.0 (Proteome Software), a program that relies on various search engine results (i.e., Sequest, X!Tandem, Mascot) and which uses Bayesian statistics to reliably identify more spectra [65]. Proteins were accepted that passed a minimum of two peptides identified at 1% peptide and protein false discovery rate (FDR), within Scaffold. Proteins with >20 peptides identified were included in the heatmap of spectral counts. For the correlation between IP/MS and FACS/RNA-seq datasets, there was no filter applied and the IP duplicate spectral counts were plotted for each gene ID.

FACS and RNA-seq Cells were stained with saturating amounts of Alexa Fluor 647-labeled IgG1s (5 to 75 μg/mL) as outlined in the Flow Cytometry section and 40 ng/mL DAPI. Viable cells were gated for target negative/low and positive/high populations, with gates drawn to keep at least 10-fold separation in fluorescence intensity, resulting in 1-5% in each population. The resulting cells were washed in DPBS and cultured for 1 week in X-Vivo medium (Lonza, contains gentamycin) supplemented with 10% FBS and 1×PS. After the first week, cells were cultured using standard culture medium (as specified in Cell lines and primary cells). Stable differential expression was verified after at least 3 passages using flow cytometry. For RNA preparation, 5×106 cells were harvested, washed once with DPBS, and lysed with 600 μL of PureLink RNA Lysis Buffer (Thermo Fisher Scientific), supplemented with 1% 2-mercaptoethanol. RNA purification was conducted according to the manufacturer's protocol, with the added on-column DNase treatment step. RNA samples were prepared in triplicate for each sample, total RNA was quantified using the Qubit 2.0 Fluorometer (Thermo Fisher Scientific), and quality was assessed using Agilent 2100 Bioanalyzer RNA Nano Chips (Agilent Technologies). All RNA samples revealed excellent quality with RNA Integrity Number (RIN) >9.3. Messenger RNA was selectively isolated from total RNA (1 μg input) using a NEBNext poly(A) mRNA Magnetic Isolation Module (NEB, #E7490) according to the manufacturer's guidelines. The library preparation from the enriched mRNA was conducted using a NEBNext Ultra II Directional RNA Library Prep Kit (NEB, #E7760). The final libraries were validated on an Agilent D1000 ScreenTape system, loaded onto a NextSeq 500 (Illumina) flow cell at 1.8 pM final concentration, and sequenced using 2×40 bp paired-end chemistry, with a targeted depth of 20-30 million reads per sample. Reads were trimmed to remove exogenous adapter sequences, low quality bases from the ends of reads, and whole reads if the average quality was too low. Processed reads were mapped to the human genome (human-ENSEMBLgrch38.r91 downloaded Feb. 16, 2018) with STAR aligner version 2.5.2a and gene abundance was estimated with HTSeq version 0.11.0 in Python version 2.7.11. Comparative RNA expression was assessed with DESeq2 versions 1.22.2 and 1.34.0 in R versions 3.5.1 and 4.1.2, respectively. For the correlation between IP/MS and FACS/RNA-seq datasets, the data were filtered using p value <0.01 and log 2(fold change) >1, then the log 2(fold change) values for the selected genes were plotted.

ELISA ELISA was conducted as described previously [32]. Briefly, 50-150 ng of antigen was coated overnight in half-area 96-well plates at 4° C., blocked with 3% BSA at 37° C. for 1 h, and 150 ng of chimeric rabbit/human IgG1 was added, incubating for 2 h at 37° C. After 3 washes with PBS, the secondary antibody, peroxidase-conjugated goat anti-human Fab diluted 1:1,000 (Jackson ImmunoResearch, #109-036-097) was added, incubated for 1 h, and washed 3 times prior to adding substrate, BioFX ABTS One Component HRP Microwell Substrate (Surmodics). After incubating at 37° C. for 15 min-1 h (depending on intensity of controls), data were acquired using a SpectraMax M5 plate reader (Molecular Devices).

siRNA Knockdown

For each prospective target, 1 μM of Accel human SMARTpool siRNA (Horizon Discovery) were added to H929 (0.5×106 cells/mL) cells in Accell Delivery Media. After 3 days, an equal volume of standard RPMI medium was added. On day 4 after transfection, target knockdown was assessed by flow cytometry using the Alexa Fluor 647-labeled and Fc-silenced IgG1s for staining.

### Surface Plasmon Resonance (SPR)

A Biacore X100 instrument was used to determine the monovalent affinities of the C-1 cluster of Fabs for recombinant PTPRG-Fc, as previously described [26]. PTPRG-Fc was immobilized using a goat anti-human Fc antibody (Jackson ImmunoResearch), which had been conjugated to a Biacore CM5 chip (Cytiva), and a series of Fab titrations were injected. Each experiment was repeated in triplicate. Rate constants (kₒₙ, k_{off}, and K_{d}) were calculated using the Biacore evaluation software (Cytiva).

### REFERENCES

1. Goydel, R. S., Rader C., (2021). Antibody-based cancer therapy. Oncogene 40, 3655-64.
2. McCafferty, J., Griffiths, A. D., Winter, G., Chiswell, D. J., (1990). Phage antibodies: filamentous phage displaying antibody variable domains. Nature 348, 552-554.
3. Barbas, C. F., III, Kang, A. S., Lerner, R. A., Benkovic, S. J., (1991). Assembly of combinatorial antibody libraries on phage surfaces: the gene III site. Proc. Natl. Acad. Sci. U.S.A. 88, 7978-7982.
4. Frenzel, A., Schirrmann, T., Hust, M., (2016). Phage display-derived human antibodies in clinical development and therapy. mAbs 8, 1177-1194.
5. Alfaleh, M. A., Jones, M. L., Howard, C. B., Mahler, S. M., (2017). Strategies for selecting membrane protein-specific antibodies using phage display with cell-based panning. Antibodies 6, 10.
6. Hagemann, U. B., Gunnarsson, L., Géraudie, S., Scheffler. U., Griep, R. A., Reiersen, H., Duncan, A. R., Kiprijanov, S. M., (2014). Fully human antagonistic antibodies against CCR4 potently inhibit cell signaling and chemotaxis. PLoS One 9, e103776.
7. Siegel, D. L., Chang, T. Y., Russell, S. L., Bunya, V. Y., (1997). Isolation of cell surface-specific human monoclonal antibodies using phage display and magnetically-activated cell sorting: applications in immunohematology. J. Immunol. Methods 206, 73-85.
8. Fransson, J., Tornberg, U. C., Borrebaeck, C. A., Carlsson, R., Frendéus, B., (2006). Rapid induction of apoptosis in B-cell lymphoma by functionally isolated human antibodies. Int. J. Cancer 119, 349-358.
9. Baskar, S., Suschak, J. M., Samija, I., Srinivasan, R., Childs, R. W., Pavletic, S. Z., Bishop, M. R., Rader, C., (2009). A human monoclonal antibody drug and target discovery platform for B-cell chronic lymphocytic leukemia based on allogeneic hematopoietic stem cell transplantation and phage display. Blood 114, 4494-4502.
10. Bausch-Fluck, D., Goldmann, U., Müller, S., van Oostrum, M., Müller, M., Schubert, O. T., Wollscheid, B., (2018). The in silico human surfaceome. Proc. Natl. Acad. Sci. U.S.A. 115, E10988-E10997.
11. Hosen, N., Matsunaga, Y., Hasegawa, K., Matsuno, H., Nakamura, Y., Makita, M., Watanabe, K., Yoshida, M., et al., (2017). The activated conformation of integrin β7 is a novel multiple myeloma-specific target for CAR T cell therapy. Nat. Med. 23, 1436-1443.
12. Büll, C., Stoel, M. A., den Brok, M. H., Adema, G. J., (2014). Sialic acids sweeten a tumor's life. Cancer Res. 74, 3199-3204.
13. Flynn, R. A., Pedram, K., Malaker, S. A., Batista, P. J., Smith, B. A. H., Johnson, A. G., George, B. M., Majzoub, K., et al., (2021). Small RNAs are modified with N-glycans and displayed on the surface of living cells. Cell 184, 3109-3124.
14. Hoogenboom, H. R., de Bruïne, A. P., Hufton, S. E., Hoet, R. M., Arends, J. W., Roovers, R. C., (1998). Antibody phage display technology and its applications. Immunotechnology 4, 1-20.
15. Derda, R., Tang, S. K., Li, S. C., Ng, S., Matochko, W., Jafari, M. R., (2011). Diversity of phage-displayed libraries of peptides during panning and amplification. Molecules 16, 1776-1803.
16. Wilson, H. D., Li, X., Peng, H., Rader, C., (2018). A Sortase A programmable phage display format for improved panning of Fab antibody libraries. J. Mol. Biol. 430, 4387-4400.
17. Glanville, J., D'Angelo, S., Khan, T. A., Reddy, S. T., Naranjo, L., Ferrara, F., Bradbury, A. R. M., (2015). Deep sequencing in library selection projects: what insight does it bring? Curr. Opin. Struct. Biol. 33, 146-160.
18. Ferrara, F., Teixeira, A. A., Naranjo, L., Erasmus, M. F., D'Angelo, S., Bradbury, A. R. M., (2020). Exploiting next-generation sequencing in antibody selections—a simple PCR method to recover binders. mAbs 12, 1701792.
19. Rouet, R., Jackson, K. J. L., Langley, D. B., Christ, D., (2018). Next-generation sequencing of antibody display repertoires. Front. Immunol. 9, 118.
20. Jones, M. L., Alfaleh, M. A., Kumble, S., Zhang, S., Osborne, G. W., Yeh, M., Arora, N., Hou, J. J. C., et al., (2016). Targeting membrane proteins for antibody discovery using phage display. Sci. Rep. 6, 26240.
21. Zhang, H., Torkamani, A., Jones, T. M., Ruiz, D. I., Pons, J., Lerner, R. A., (2011). Phenotype-information-phenotype cycle for deconvolution of combinatorial antibody libraries selected against complex systems. Proc. Natl. Acad. Sci. U.S.A. 108, 13456-13461.
22. Deschaght, P., Vintém, A. P., Logghe, M., Conde, M., Felix, D., Mensink, R., Gonçalves, J., Audiens, J., et al., (2017). Large diversity of functional nanobodies from a camelid immune library revealed by an alternative analysis of next-generation sequencing data. Front. Immunol. 8:420.
23. Freeth, J., Soden, J., (2020). New advances in cell microarray technology to expand applications in target deconvolution and off-target screening. SLAS Discov. 25, 223-230.
24. Huston-Paterson, D. J., Banik, S. S. R., Doranz, B. J., (2016). Screening the membrane protein: a high-throughput platform for identifying membrane protein antibody targets. Genetic Engineering & Biotechnology News 36:(15) 18-19.
25. Ferrara, F., Bradbury, A. R. M., D'Angelo, S., (2018). Primer design and inverse PCR on yeast display antibody selection outputs. Methods Mol. Biol. 1721, 35-45.
26. Peng, H., Nerreter, T., Chang, J., Qi, J., Li, X., Karunadharma, P., Martinez, G. J., Fallahi, M., et al., (2017). Mining naive rabbit antibody repertoires by phage display for monoclonal antibodies of therapeutic utility. J. Mol. Biol. 429, 2954-2973.
27. Ruiz-Argüelles, G. J., San Miguel, J. F., (1994). Cell surface markers in multiple myeloma. Mayo Clin. Proc. 69, 684-690.
28. Huang, Y., Niu, B., Gao, Y., Fu, L., Li, W., (2010). CD-HIT Suite: a web server for clustering and comparing biological sequences. Bioinformatics 26, 680-682.
29. Spiliotopoulos, A., Owen, J. P., Maddison, B. C., Dreveny, I., Rees, H. C., Gough, K. C., (2015). Sensitive recovery of recombinant antibody clones after their in silico identification within NGS datasets. J. Immunol. Methods 420, 50-55.
30. Quan, S., Hiniker, A., Collet, J. F., Bardwell, J. C., (2013). Isolation of bacteria envelope proteins. Methods Mol. Biol. 966, 359-366.
31. Schlothauer, T., Herter, S., Koller, C. F., Grau-Richards, S., Steinhart, V., Spick, C., Kubbies, M., Klein, C., et al., (2016). Novel human IgG1 and IgG4 Fc-engineered antibodies with completely abolished immune effector functions. Protein Eng. Des. Sel. 29, 457-466.
32. Chang, J., Peng, H., Shaffer, B. C., Baskar, S., Wecken, I. C., Cyr, M. G., Martinez, G. J., Soden, J., et al., (2018). Siglec-6 on chronic lymphocytic leukemia cells is a target for post-allogeneic hematopoietic stem cell transplantation antibodies. Cancer Immunol. Res. 6, 1008-1013.
33. Wichert, S., Juliusson, G., Johansson, Å., Sonesson, E., Teige, I., Wickenberg, A. T., Frendéus, B., Korsgren, M., et al., (2017). A single-arm, open-label, phase 2 clinical trial evaluating disease response following treatment with BI-505, a human anti-intercellular adhesion molecule-1 monoclonal antibody, in patients with smoldering multiple myeloma. PLoS One 12, e0171205.
34. Uhlen, M., Oksvold, P., Fagerberg, L., Lundberg, E., Jonasson, K., Forsberg, M., Zwahlen, M., Kampf, C., et al., (2010). Towards a knowledge-based Human Protein Atlas. Nat. Biotechnol. 28, 1248-1250.
35. Kikuchi, S., Iwai, M., Sakurai-Yageta, M., Tsuboi, Y., Ito, T., Maruyama, T., Tsuda, H., Kanai, Y., et al., (2012). Expression of a splicing variant of the CADM1 specific to small cell lung cancer. Cancer Sci. 103, 1051-1057.
36. Walker, B. A., Wardell, C. P., Melchor, L., Hulkki, S., Potter, N. E., Johnson, D. C., Fenwick, K., Kozarewa, I., et al., (2012). Intraclonal heterogeneity and distinct molecular mechanisms characterize the development of t(4;14) and t(11;14) myeloma. Blood 120, 1077-1086.
37. Guo, M., Schimmel, P., (2013). Essential nontranslational functions of tRNA synthetases. Nat. Chem. Biol. 9, 145-153.
38. Park, M. C., Kang, T., Jin, D., Han, J. M., Kim, S. B., Park, Y. J., Cho, K., Park, Y., et al., (2012). Secreted human glycyl-tRNA synthetase implicated in defense against ERK-activated tumorigenesis. Proc. Natl. Acad. Sci. U.S.A. 109, E640-E647.
39. Barbas, C. F., III, Hu, D., Dunlop, N., Sawyer, L., Cababa, D., Hendry, R. M., Nara, P. L., Burton, D. R., (1994). In vitro evolution of a neutralizing human antibody to human immunodeficiency virus type 1 to enhance affinity and broaden strain cross-reactivity. Proc. Natl. Acad. Sci. U.S.A. 91, 3809-3813.
40. Goydel, R. S., Weber, J., Peng, H., Qi, J., Soden, J., Freeth, J., Park, H., Rader, C., (2020). Affinity maturation, humanization, and co-crystallization of a rabbit anti-human ROR2 monoclonal antibody for therapeutic applications. J. Biol. Chem. 295, 5995-6006.
41. Morrison, M. S., Wang, T., Raguram, A., Hemez, C., Liu, D. R., (2021). Disulfide-compatible phage-assisted continuous evolution in the periplasmic space. Nat. Commun. 12, 5959.
42. Choe, J. H., Watchmaker, P. B., Simic, M. S., Gilbert, R. D., Li, A. W., Krasnow, N. A., Downey, K. M., Yu, W., et al., (2021). SynNotch-CAR T cells overcome challenges of specificity, heterogeneity, and persistence in treating glioblastoma. Sci. Transl. Med. 13 (591), eabe7378.
43. Oostindie, S. C., Lazar, G. A., Schuurman, J., Parren, P. W. H. I., (2022). Avidity in antibody effector functions and biotherapeutic drug design. Nat. Rev. Drug Discov. 21, 715-735.
44. Veitonmäki, N., Hansson, M., Zhana, F., Sundberg, A., Löfstedt, T., Ljungars, A., Zi, Z. C., Martinsson-Niskanen, T., et al., (2013). A human ICAM-1 antibody isolated by a function-first approach has potent macrophage-dependent antimyeloma activity in vivo. Cancer Cell 23, 502-515.
45. Kurosawa, G., Akahori, Y., Morita, M., Sumitomo, M., Sato, N., Muramatsu, C., Eguchi, K., Matsuda, K., et al., (2008). Comprehensive screening for antigens overexpressed on carcinomas via isolation of human mAbs that may be therapeutic. Proc. Natl. Acad. Sci. U.S.A. 105, 7287-7292.
46. de Fougerolles, A. R., Springer, T. A., (1992). Intercellular adhesion molecule 3, a third adhesion counter-receptor for lymphocyte function-associated molecule 1 on resting lymphocytes. J. Exp. Med. 175, 185-190.
47. Cyr, M. G., Mhibik, M., Qi, J., Peng, H., Chang, J., Gaglione, E. M., Eik, D., Herrick, J., et al., (2022). Patient-derived Siglec-6-targeting antibodies engineered for T-cell recruitment have potential therapeutic utility in chronic lymphocytic leukemia. J. Immunother. Cancer 10, e004850.
48. Xiao, H., Woods, E. C., Vukojicic, P., Bertozzi, C. R., (2016). Precision glycocalyx editing as a strategy for cancer immunotherapy. Proc. Natl. Acad. Sci. U.S.A. 113, 10304-10309.
49. Möckl, L., (2020). The emerging role of the mammalian glycocalyx in functional membrane organization and immune system regulation. Front. Cell Dev. Biol. 8, 253.
50. Alt, F. W., Blackwell, T. K., Yancopoulos, G. D., (1987). Development of the primary antibody repertoire. Science 238, 1079-1087.
51. Tonegawa, S., (1983). Somatic generation of antibody diversity. Nature 302, 575-581.
52. Xu, J. L., Davis, M. M., (2000). Diversity in the CDR3 region of V(H) is sufficient for most antibody specificities. Immunity 13, 37-45.
53. Al-Lazikani, B., Lesk, A. M., Chothia, C., (1997). Standard conformations for the canonical structures of immunoglobulins. J. Mol. Biol. 273, 927-948.
54. Quail, M. A., Smith, M., Coupland, P., Otto, T. D., Harris, S. R., Connor, T. R., Bertoni, A., Swerdlow, H. P., et al., (2012). A tale of three next generation sequencing platforms: comparison of Ion Torrent, Pacific Biosciences and Illumina MiSeq sequencers. BMC Genomics 13, 341.
55. Jain, M., Olsen, H. E., Paten, B., Akeson, M., (2016). The Oxford Nanopore MinION: delivery of sequencing to the genomics community. Genome Biol. 17, 239.
56. Yang, W., Yoon, A., Lee, S., Kim, S., Han, J., Chung, J., (2017). Next-generation sequencing enables the discovery of more diverse positive clones from a phage-displayed antibody library. Exp. Mol. Med. 49, e308.
57. Jo, M., Jung, S. T., (2016). Engineering therapeutic antibodies targeting G-protein-coupled receptors. Exp. Mol. Med. 48, e207.
58. Hauser, A. S., Attwood, M. M., Rask-Andersen, M., Schiöth, H. B., Gloriam, D. E., (2017). Trends in GPCR drug discovery: new agents, targets and indications. Nat. Rev. Drug Discov. 16, 829-842.
59. Ayoub, M. A., Crépieux, P., Koglin, M., Parmentier, M., Pin, J.-P., Poupon, A., Reiter, E., Smit, M., et al., (2017). Antibodies targeting G protein-coupled receptors: Recent advances and therapeutic challenges. mAbs 9, 735-741.
60. Gerber, H. P., Presta, L. G., (2022). TCR mimic compounds for pHLA targeting with high potency modalities in oncology. Front. Oncol. 12, 1027548.
61. Wallace, R. B., Shaffer, J., Murphy, R. F., Bonner, J., Hirose, T., Itakura, K., (1979). Hybridization of synthetic oligodeoxyribonucleotides to phi chi 174 DNA: the effect of single base pair mismatch. Nucleic Acids Res. 6, 3543-3557.
62. Kwong, K. Y., Rader, C., (2009). E. coli expression and purification of Fab antibody fragments. Curr. Protoc. Protein Sci. 6, 10.
63. Lytvynenko, I., Paternoga, H., Thrun, A., Balke, A., Müller, T. A., Chiang, C. H., Nagler, K., Tsaprailis, G., et al., (2019). Alanine tails signal proteolysis in bacterial ribosome-associated quality control. Cell 178, 76-90.
64. Qian, W. J., Liu, T., Monroe, M. E., Strittmatter, E. F., Jacobs, J. M., Kangas, L. J., Petritis, K., Camp, D. G., II, et al., (2005). Probability-based evaluation of peptide and protein identifications from tandem mass spectrometry and SEQUEST analysis: the human proteome. J. Proteome Res. 4, 53-62.
65. Keller, A., Nesvizhskii, A. I., Kolker, E., Aebersold, R., (2002). Empirical statistical model to estimate the accuracy of peptide identifications made by MS/MS and database search. Anal. Chem. 74, 5383-5392.
66. The Antibody Society. Therapeutic monoclonal antibodies approved or in review in the EU or US. [cited 2022 April 19]. Available from: WorldWideWeb antibodysociety.org/resources/approved-antibodies.
67. Lerner R A. Combinatorial antibody libraries: new advances, new immunological insights. Nat Rev Immunol. 2016; 16(8):498-508. doi: 10.1038/nri.2016.67. PubMed PMID: 27374636.
68. Li W, Godzik A. Cd-hit: a fast program for clustering and comparing large sets of protein or nucleotide sequences. Bioinformatics. 2006; 22(13):1658-9. doi: 10.1093/bioinformatics/btl158. PubMed PMID: 16731699.
69. Edgar R C. Search and clustering orders of magnitude faster than BLAST. Bioinformatics. 2010; 26(19):2460-1. doi: 10.1093/bioinformatics/btq461. PubMed PMID: 20709691.
70. Ye J, Ma N, Madden T L, Ostell J M. IgBLAST: an immunoglobulin variable domain sequence analysis tool. Nucleic acids research. 2013; 41(Web Server issue):W34-40. doi: 10.1093/nar/gkt382. PubMed PMID: 23671333.
71. Aouinti S, Malouche D, Giudicelli V, Kossida S, Lefranc M-P. IMGT/HighV-QUEST Statistical Significance of IMGT Clonotype (AA) Diversity per Gene for Standardized Comparisons of Next Generation Sequencing Immunoprofiles of Immunoglobulins and T Cell Receptors. PLoS One. 2015; 10(11):e0142353-e. doi: 10.1371/journal.pone.0142353. PubMed PMID: 26540440.
72. Aouinti S, Giudicelli V, Duroux P. Malouche D, Kossida S, Lefranc M-P. IMGT/StatClonotype for Pairwise Evaluation and Visualization of NGS IG and TR IMGT Clonotype (AA) Diversity or Expression from IMGT/HighV-QUEST. Frontiers in immunology. 2016; 7:339-. doi: 10.3389/fimmu.2016.00339. PubMed PMID: 27667992.
73. Lefranc M-P, Giudicelli V, Ginestoux C, Bodmer J, Müller W, Bontrop R, Lemaitre M, Malik A, Barbié V, Chaume D. IMGT, the international ImMunoGeneTics database. Nucleic Acids Res. 1999; 27(1):209-12. doi: 10.1093/nar/27.1.209.
74. D'Angelo S, Glanville J, Ferrara F, Naranjo L, Gleasner C D, Shen X, Bradbury A R M, Kiss C. The antibody mining toolbox: an open source tool for the rapid analysis of antibody repertoires. mAbs. 2014; 6(1):160-72. doi: 10.4161/mabs.27105. PubMed PMID: 24423623.
75. Love M I, Huber W, Anders S. Moderated estimation of fold change and dispersion for RNA-seq data with DESeq2. Genome biology. 2014; 15(12):550. doi: 10.1186/s13059-014-0550-8. PubMed PMID: 25516281.
76. Shabaneh T B, Downey S L, Goddard A L, Screen M, Lucas M M, Eastman A, Kisselev A F. Molecular basis of differential sensitivity of myeloma cells to clinically relevant bolus treatment with bortezomib. PLoS One. 2013; 8(2):e56132. doi: 10.1371/journal.pone.0056132. PubMed PMID: 23460792.
77. Gooding R P, Bybee A, Cooke F, Little A, Marsh S G, Coelho E, Gupta D, Samson D, Apperley J F. Phenotypic and molecular analysis of six human cell lines derived from patients with plasma cell dyscrasia. Br J Haematol. 1999; 106(3):669-81. PubMed PMID: 10468855.
78. Muccio V E, Saraci E, Gilestro M, Gattei V, Zucchetto A, Astolfi M, Ruggeri M, Marzanati E, Passera R, Palumbo A, Boccadoro M, Omede P. Multiple myeloma: New surface antigens for the characterization of plasma cells in the era of novel agents. Cytometry Part B, Clinical cytometry. 2016; 90(1):81-90. doi: 10.1002/cyto.b.21279. PubMed PMID: 26287276.
79. Ahn I E, Brown J R. Targeting Bruton's Tyrosine Kinase in CLL. Front Immunol. 2021; 12:687458. doi: 10.3389/fimmu.2021.687458. PubMed PMID: 34248972.
80. Hemadou A, Giudicelli V, Smith M L, Lefranc M-P, Duroux P, Kossida S, Heiner C, Hepler N L, Kuijpers J, Groppi A, Korlach J, Mondon P, Ottones F, Jacobin-Valat M-J, Laroche-Traineau J, Clofent-Sanchez G. Pacific Biosciences Sequencing and IMGT/HighV-QUEST Analysis of Full-Length Single Chain Fragment Variable from an In Vivo Selected Phage-Display Combinatorial Library. Frontiers in immunology. 2017; 8:1796-. doi: 10.3389/fimmu.2017.01796. PubMed PMID: 29326697.
81. D'Angelo S, Ferrara F, Naranjo L, Erasmus M F, Hraber P, Bradbury A R M. Many Routes to an Antibody Heavy-Chain CDR3: Necessary, Yet Insufficient, for Specific Binding. Front Immunol. 2018; 9:395. doi: 10.3389/fimmu.2018.00395. PubMed PMID: 29568296.
82. Ravn U, Gueneau F, Baerlocher L, Osteras M, Desmurs M, Malinge P, Magistrelli G, Farinelli L, Kosco-Vilbois M H, Fischer N. By-passing in vitro screening—next generation sequencing technologies applied to antibody display and in silico candidate selection. Nucleic acids research. 2010; 38(21):e193. doi: 10.1093/nar/gkg789. ubMed PMID: 20846958.
83. Sarin V, Yu K, Ferguson I D, Gugliemini O, Nix M A, Hann B, Sirota M, Wiita A P. Evaluating the efficacy of multiple myeloma cell lines as models for patient tumors via transcriptomic correlation analysis. Leukemia. 2020; 34(10):2754-65. doi: 10.1038/s41375-020-0785-1. PubMed PMID: 32123307.
84. Schlothauer T, Herter S, Koller C F, Grau-Richards S, Steinhart V, Spick C, Kubbies M, Klein C, Umana P, Mossner E. Novel human IgG1 and IgG4 Fc-engineered antibodies with completely abolished immune effector functions. Protein Eng Des Sel. 2016; 29(10):457-66. doi: 10.1093/protein/gzw040. PubMed PMID: 27578889.
85. Lavinder J J, Hoi K H, Reddy S T, Wine Y, Georgiou G. Systematic Characterization and Comparative Analysis of the Rabbit Immunoglobulin Repertoire. PLoS One. 2014; 9(6):e101322. doi: 10.1371/journal.pone.0101322. PubMed PMID: PMC4076286.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, UniProt sequences, patents, and patent applications cited in this specification are herein incorporated by reference as if each was specifically and individually indicated to be incorporated by reference.

1. An isolated antibody that competes for binding to a multiple myeloma cell surface antigen with antibody HW-25, HW-1, HW-42, HW-70, HW-17, HW-8, HW-16, HW-45, HW-28, HW-56, HW-81, HW-97, HW-101, or HW-113.

2. (canceled)

3. The antibody of claim 1, wherein the multiple myeloma cell surface antigen is PTPRG, ICAM1, CADM1, or GARS.

4. The antibody of claim 3 specifically binding PTPRG
comprising three heavy chain CDRs and three light chain CDRs of antibody HW-25, wherein HW-25 is characterized by a mature heavy chain variable region having an amino acid sequence comprising SEQ ID NO:31 and a mature light chain variable region having an amino acid sequence comprising SEQ ID NO:32;
comprising three heavy chain CDRs and three light chain CDRs of antibody HW-1, wherein HW-1 is characterized by a mature heavy chain variable region having an amino acid sequence comprising SEQ ID NO:41 and a mature light chain variable region having an amino acid sequence comprising SEQ ID NO:42;
comprising three heavy chain CDRs and three light chain CDRs of antibody HW-42, wherein HW-42 is characterized by a mature heavy chain variable region having an amino acid sequence comprising SEQ ID NO:11 and a mature light chain variable region having an amino acid sequence comprising SEQ ID NO:12;
comprising three heavy chain CDRs and three light chain CDRs of antibody HW-70, wherein HW-70 is characterized by a mature heavy chain variable region having an amino acid sequence comprising SEQ ID NO:21 and a mature light chain variable region having an amino acid sequence comprising SEQ ID NO:22; or
comprising three heavy chain CDRs and three light chain CDRs of antibody HW-17, wherein HW-17 is characterized by a mature heavy chain variable region having an amino acid sequence comprising SEQ ID NO:51 and a mature light chain variable region having an amino acid sequence comprising SEQ ID NO:52.

5. The antibody of claim 4, wherein the CDRs are of a definition selected from the group consisting of Kabat, Chothia, Kabat/Chothia Composite, AbM, Contact, and IMGT.

6. The antibody of claim 5,
wherein the mature heavy chain variable region comprises the three IMGT heavy chain CDRs of SEQ ID NOs:35-37 and the mature light chain variable region comprises the three IMGT light chain CDRs of SEQ ID NOs:38-40;
wherein the mature heavy chain variable region comprises the three IMGT heavy chain CDRs of SEQ ID NOs:45-47 and the mature light chain variable region comprises the three IMGT light chain CDRs of SEQ ID NOs:48-50;
wherein the mature heavy chain variable region comprises the three IMGT heavy chain CDRs of SEQ ID NOs:15-17 and the mature light chain variable region comprises the three IMGT light chain CDRs of SEQ ID NOs:18-20;
wherein the mature heavy chain variable region comprises the three IMGT heavy chain CDRs of SEQ ID NOs:25-27 and the mature light chain variable region comprises the three IMGT light chain CDRs of SEQ ID NOs:28-30; or
wherein the mature heavy chain variable region comprises the three IMGT heavy chain CDRs of SEQ ID NOs:55-57 and the mature light chain variable region comprises the three IMGT light chain CDRs of SEQ ID NOs:58-60.

7. The antibody of claim 6,
comprising a mature heavy chain variable region having an amino acid sequence at least 90% identical to SEQ ID NO:31, and a mature light chain variable region having an amino acid sequence at least 90% identical to SEQ ID NO:32;
comprising a mature heavy chain variable region having an amino acid sequence at least 90% identical to SEQ ID NO:41, and a mature light chain variable region having an amino acid sequence at least 90% identical to SEQ ID NO:42;
comprising a mature heavy chain variable region having an amino acid sequence at least 90% identical to SEQ ID NO:11, and a mature light chain variable region having an amino acid sequence at least 90% identical to SEQ ID NO:12;
comprising a mature heavy chain variable region having an amino acid sequence at least 90% identical to SEQ ID NO:21, and a mature light chain variable region having an amino acid sequence at least 90% identical to SEQ ID NO:22; or
comprising a mature heavy chain variable region having an amino acid sequence at least 90% identical to SEQ ID NO:51, and a mature light chain variable region having an amino acid sequence at least 90% identical to SEQ ID NO:52.

8. The antibody of claim 7,
wherein the mature heavy chain variable region has an amino acid sequence of SEQ ID NO:31 and the mature light chain variable region has an amino acid sequence of SEQ ID NO:32;
wherein the mature heavy chain variable region has an amino acid sequence of SEQ ID NO:41 and the mature light chain variable region has an amino acid sequence of SEQ ID NO:42;
wherein the mature heavy chain variable region has an amino acid sequence of SEQ ID NO:11 and the mature light chain variable region has an amino acid sequence of SEQ ID NO:12;
wherein the mature heavy chain variable region has an amino acid sequence of SEQ ID NO:21 and the mature light chain variable region has an amino acid sequence of SEQ ID NO:22; or
wherein the mature heavy chain variable region has an amino acid sequence of SEQ ID NO:51 and the mature light chain variable region has an amino acid sequence of SEQ ID NO:52.

9.-23. (canceled)

24. The antibody of claim 1, wherein the antibody is chimeric or humanized.

25. The antibody of claim 1, wherein the antibody is IgA1, IgA2, IgD, IgE, IgG1, IgG2, IgG3, IgG4, synthetic IgG, IgM, F(ab)2, Fv, scFv, IgGACH2, F(ab′)2, scFv2CH3, Fab, VL, VH, scFv4, scFv3, scFv2, dsFv, Fv, scFv-Fc, (scFv)2, a non-depleting IgG, a diabody, or a bivalent antibody.

26. The antibody of claim 1 wherein the mature light chain variable region is fused to a light chain constant region and the mature heavy chain variable region is fused to a heavy chain constant region.

27. (canceled)

28. The antibody of claim 26,
comprising a heavy chain of SEQ ID NO:33 and a light chain of SEQ ID NO:34;
comprising a heavy chain of SEQ ID NO:43 and a light chain of SEQ ID NO:44;
comprising a heavy chain of SEQ ID NO:13 and a light chain of SEQ ID NO:14;
comprising a heavy chain of SEQ ID NO:23 and a light chain of SEQ ID NO:24;
comprising a heavy chain of SEQ ID NO:53 and a light chain of SEQ ID NO:54;
comprising a heavy chain of SEQ ID NO:63 and a light chain of SEQ ID NO:64;
comprising a heavy chain of SEQ ID NO:73 and a light chain of SEQ ID NO:74;
comprising a heavy chain of SEQ ID NO:83 and a light chain of SEQ ID NO:84;
comprising a heavy chain of SEQ ID NO:93 and a light chain of SEQ ID NO:94;
comprising a heavy chain of SEQ ID NO:103 and a light chain of SEQ ID NO:104;
comprising a heavy chain of SEQ ID NO:113 and a light chain of SEQ ID NO:114;
comprising a heavy chain of SEQ ID NO:123 and a light chain of SEQ ID NO:124;
comprising a heavy chain of SEQ ID NO:133 and a light chain of SEQ ID NO:134; or
comprising a heavy chain of SEQ ID NO:143 and a light chain of SEQ ID NO:144.

29. The antibody of claim 1, which is an antibody fragment or is a bispecific antibody.

30.-32. (canceled)

33. The antibody of claim 1, which is an antibody-based binding protein.

34. The antibody of claim 1, which is conjugated to a synthetic molecule.

35.-36. (canceled)

37. The antibody of claim 1 linked to at least one cytotoxic agent as an antibody drug conjugate.

38.-39. (canceled)

40. A chimeric antigen receptor (CAR), comprising the antibody of claim 1 that is fused to a transmembrane region and an intracellular T-cell receptor (TCR) signaling domain.

41. (canceled)

42. A pharmaceutical composition comprising a therapeutically effective amount of the antibody of claim 1 and a pharmaceutically acceptable carrier.

43. (canceled)

44. A nucleic acid encoding the heavy chain and/or light chain of an antibody as described in claim 1.

45. (canceled)

46. A method of killing or inhibiting the growth of a cell expressing a multiple myeloma cell surface antigen in a subject, comprising administering a pharmaceutical composition of claim 42 to a subject in need thereof, thereby killing or inhibiting the growth of the cell expressing the multiple myeloma cell surface antigen in the subject.

47.-49. (canceled)

50. A method of treating a hematologic malignancy in a subject, comprising administering a pharmaceutical composition of claim 42 to the subject having the hematologic malignancy, thereby treating the hematologic malignancy in the subject.

51.-54. (canceled)


SEQUENCE LISTING (174 sequences)

SEQ ID NO: 1 - protein, 1445 aa, Homo sapiens
MRRLLEPCWWILFLKITSSVLHYVVCFPALTEGYVGALHENRHGSAVQIRRRKASGDPYWAYSGAYGPEHWVTSSVSCGGRHQSPIDILDQYARVGEEYQELQLDGFDNESSNKTWMKNTGKTVAILLKDDYFVSGAGLPGRFKAEKVEFHWGHSNGSAGSEHSINGRRFPVEMQIFFYNPDDFDSFQTAISENRIIGAMAIFFQVSPRDNSALDPIIHGLKGVVHHEKETFLDPFVLRDLLPASLGSYYRYTGSLTTPPCSEIVEWIVFRRPVPISYHQLEAFYSIFTTEQQDHVKSVEYLRNNFRPQQRLHDRVVSKSAVRDSWNHDMTDFLENPLGTEASKVCSSPPIHMKVQPLNQTALQVSWSQPETIYHPPIMNYMISYSWTKNEDEKEKTFTKDSDKDLKATISHVSPDSLYLFRVQAVCRNDMRSDFSQTMLFQANTTRIFQGTRIVKTGVPTASPASSADMAPISSGSSTWTSSGIPFSFVSMATGMGPSSSGSQATVASVVTSTLLAGLGFGGGGISSFPSTVWPTRLPTAASASKQAARPVLATTEALASPGPDGDSSPTKDGEGTEEGEKDEKSESEDGEREHEEDGEKDSEKKEKSGVTHAAEERNQTEPSPTPSSPNRTAEGGHQTIPGHEQDHTAVPTDQTGGRRDAGPGLDPDMVTSTQVPPTATEEQYAGSDPKRPEMPSKKPMSRGDRFSEDSRFITVNPAEKNTSGMISRPAPGRMEWIIPLIVVSALTFVCLILLIAVLVYWRGCNKIKSKGFPRRFREVPSSGERGEKGSRKCFQTAHFYVEDSSSPRVVPNESIPIIPIPDDMEAIPVKQFVKHIGELYSNNQHGFSEDFEEVQRCTADMNITAEHSNHPENKHKNRYINILAYDHSRVKLRPLPGKDSKHSDYINANYVDGYNKAKAYIATQGPLKSTFEDFWRMIWEQNTGIIVMITNLVEKGRRKCDQYWPTENSEEYGNIIVTLKSTKIHACYTVRRFSIRNTKVKKGQKGNPKGRQNERVVIQYHYTQWPDMGVPEYALPVLTFVRRSSAARMPETGPVLVHCSAGVGRTGTYIVIDSMLQQIKDKSTVNVLGFLKHIRTQRNYLVQTEEQYIFIHDALLEAILGKETEVSSNQLHSYVNSILIPGVGGKTRLEKQFKLVTQCNAKYVECFSAQKECNKEKNRNSSVVPSERARVGLAPLPGMKGTDYINASYIMGYYRSNEFIITQHPLPHTTKDFWRMIWDHNAQIIVMLPDNQSLAEDEFVYWPSREESMNCEAFTVTLISKDRLCLSNEEQIIIHDFILEATQDDYVLEVRHFQCPKWPNPDAPISSTFELINVIKEEALTRDGPTIVHDEYGAVSAGMLCALTTLSQQLENENAVDVFQVAKMINLMRPGVFTDIEQYQFIYKAMLSLVSTKENGNGPMTVDKNGAVLIADESDPAESMESLV

SEQ ID NO: 2 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 3 - protein, 442 aa, Homo sapiens
MASVVLPSGSQCAAAAAAAAPPGLRLRLLLLLFSAAALIPTGDGQNLFTKDVTVIEGEVATISCQVNKSDDSVIQLLNPNRQTIYFRDFRPLKDSRFQLLNFSSSELKVSLTNVSISDEGRYFCQLYTDPPQESYTTITVLVPPRNLMIDIQKDTAVEGEEIEVNCTAMASKPATTIRWFKGNTELKGKSEVEEWSDMYTVTSQLMLKVHKEDDGVPVICQVEHPAVTGNLQTQRYLEVQYKPQVHIQMTYPLQGLTREGDALELTCEAIGKPQPVMVTWVRVDDEMPQHAVLSGPNLFINNLNKTDNGTYRCEASNIVGKAHSDYMLYVYDPPTTIPPPTTTTTTTTTTTTTILTIITDSRAGEEGSIRAVDHAVIGGVVAVVVFAMLCLLIILGRYFARHKGTYFTHEAKGADDAADADTAIINAEGGQNNSEEKKEYFI

SEQ ID NO: 4 - protein, 739 aa, Homo sapiens
MPSPRPVLLRGARAALLLLLPPRLLARPSLLLRRSLSAASCPPISLPAAASRSSMDGAGAEEVLAPLRLAVRQQGDLVRKLKEDKAPQVDVDKAVAELKARKRVLEAKELALQPKDDIVDRAKMEDTLKRRFFYDQAFAIYGGVSGLYDFGPVGCALKNNIIQTWRQHFIQEEQILEIDCTMLTPEPVLKTSGHVDKFADFMVKDVKNGECFRADHLLKAHLQKLMSDKKCSVEKKSEMESVLAQLDNYGQQELADLFVNYNVKSPITGNDLSPPVSFNLMFKTFIGPGGNMPGYLRPETAQGIFLNFKRLLEFNQGKLPFAAAQIGNSFRNEISPRSGLIRVREFTMAEIEHFVDPSEKDHPKFQNVADLHLYLYSAKAQVSGQSARKMRLGDAVEQGVINNTVLGYFIGRIYLYLTKVGISPDKLRFRQHMENEMAHYACDCWDAESKTSYGWIEIVGCADRSCYDLSCHARATKVPLVAEKPLKEPKTVNVVQFEPSKGAIGKAYKKDAKLVMEYLAICDECYITEMEMLLNEKGEFTIETEGKTFQLTKDMINVKRFQKTLYVEEVVPNVIEPSFGLGRIMYTVFEHTFHVREGDEQRTFFSFPAVVAPFKCSVLPLSQNQEFMPFVKELSEALTRHGVSHKVDDSSGSIGRRYARTDEIGVAFGVTIDFDTVNKTPHTATLRDRDSMRQIRAEISELPSIVQDLANGNITWADVEARYPLFEGQETGKKETIEE

SEQ ID NO: 5 - protein, 122 aa, synthetic construct
QSLEESGGRLVTPGTPLTLTCTVSGFSLSSYAMNWVRQAPGKGLEWIGIINSYGSTYYASWAKGRFTISKTSTTVDLKITSPTTEDTATYFCARVTYDSYGYGYAPYGMDLWGPGTLVTVSS

SEQ ID NO: 6 - protein, 107 aa, synthetic construct
LVMTQTPSSTSNAVGGTVTIKCQASQSIGNVLAWYQQKPGQPPKLLIYAASTLASGVPSRVGGSRSGTEYTLTISGVQREDAATYYCLGSDSDSDIAFGGGTELEIL

SEQ ID NO: 7 - protein, 471 aa, synthetic construct
MDWTWRILFLVAAATGAHSQSLEESGGRLVTPGTPLTLTCTVSGFSLSSYAMNWVRQAPGKGLEWIGIINSYGSTYYASWAKGRFTISKTSTTVDLKITSPTTEDTATYFCARVTYDSYGYGYAPYGMDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 8 - protein, 233 aa, synthetic construct
MDWTWRILFLVAAATGAHSLVMTQTPSSTSNAVGGTVTIKCQASQSIGNVLAWYQQKPGQPPKLLIYAASTLASGVPSRVGGSRSGTEYTLTISGVQREDAATYYCLGSDSDSDIAFGGGTELEILRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 19 aa, synthetic construct
ARVTYDSYGYGYAPYGMDL

SEQ ID NO: 10 - protein, 10 aa, synthetic construct
LGSDSDSDIA

SEQ ID NO: 11 - protein, 119 aa, synthetic construct
QSVKESEGGLFKPADTLTLTCTVSGFSLSSNEISWVRQAPGEGLEYIGIIDTGGRAYYAVWARSRSTITRNTNLNTVTLKMTSLTAADTATYFCARGGVDTEWYAFSSWGPGTLVTVPS

SEQ ID NO: 12 - protein, 110 aa, synthetic construct
SFVLTQPASVQVNLGQTVSLTCTADTLSRRYASWYQQKPGQAPVLLIFRDTSRPSGVPDRFSGSSSGDTATLTISGAQAGDEADYYCATSDGSGSNYQYVFGGGTQLTVT

SEQ ID NO: 13 - protein, 468 aa, synthetic construct
MDWTWRILFLVAAATGAHSQSVKESEGGLFKPADTLTLTCTVSGFSLSSNEISWVRQAPGEGLEYIGIIDTGGRAYYAVWARSRSTITRNTNLNTVTLKMTSLTAADTATYFCARGGVDTEWYAFSSWGPGTLVTVPSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 14 - protein, 236 aa, synthetic construct
MDWTWRILFLVAAATGAHSSFVLTQPASVQVNLGQTVSLTCTADTLSRRYASWYQQKPGQAPVLLIFRDTSRPSGVPDRFSGSSSGDTATLTISGAQAGDEADYYCATSDGSGSNYQYVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 15 - protein, 8 aa, synthetic construct
GFSLSSNE

SEQ ID NO: 16 - protein, 7 aa, synthetic construct
IDTGGRA

SEQ ID NO: 17 - protein, 14 aa, synthetic construct
ARGGVDTEWYAFSS

SEQ ID NO: 18 - protein, 6 aa, synthetic construct
TLSRRY

SEQ ID NO: 19 - skipped

SEQ ID NO: 20 - protein, 13 aa, synthetic construct
ATSDGSGSNYQYV

SEQ ID NO: 21 - protein, 113 aa, synthetic construct
QSLEESGGRLVTPGTPLTLTCTVSGFSLSSNAMSWVRQAPGKGLEWIGTIDGYGYTYYASWAKGRFTVSKTSTTVDLKITSPTTEDTATYFCAREIYLGSNFWGPGTLVTISS

SEQ ID NO: 22 - protein, 110 aa, synthetic construct
SFVLTQPASVQVNLGQTVSLTCTADTLSRRYASWYQQKPGQAPVLLIYRDTSRPSGVPDRFSGSSSGNTATLTISGAQAGDEADYYCATADGSGSSYQFVFGGGTQLTVT

SEQ ID NO: 23 - protein, 462 aa, synthetic construct
MDWTWRILFLVAAATGAHSQSLEESGGRLVTPGTPLTLTCTVSGFSLSSNAMSWVRQAPGKGLEWIGTIDGYGYTYYASWAKGRFTVSKTSTTVDLKITSPTTEDTATYFCAREIYLGSNFWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 24 - protein, 236 aa, synthetic construct
MDWTWRILFLVAAATGAHSSFVLTQPASVQVNLGQTVSLTCTADTLSRRYASWYQQKPGQAPVLLIYRDTSRPSGVPDRFSGSSSGNTATLTISGAQAGDEADYYCATADGSGSSYQFVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 8 aa, synthetic construct
GFSLSSNA

SEQ ID NO: 26 - protein, 7 aa, synthetic construct
IDGYGYT

SEQ ID NO: 27 - protein, 10 aa, synthetic construct
AREIYLGSNF

SEQ ID NO: 28 - protein, 6 aa, synthetic construct
TLSRRY

SEQ ID NO: 29 - skipped

SEQ ID NO: 30 - protein, 13 aa, synthetic construct
ATADGSGSSYQFV

SEQ ID NO: 31 - protein, 116 aa, synthetic construct
QSLEESGGRLVTPGTPLTLTCTVSGFSLSSYAMGWVRQAPGKGLEWIGIIDSRGSTYYATWAKGRFTISKTSTTVDLKITSPTTEDTATYFCAREDSYYYDSFNLWGPGTLVTVSS

SEQ ID NO: 32 - protein, 110 aa, synthetic construct
SFVLTQPASVQVNLGQTVSLTCTADTLSRRYASWYQQKPGQAPVLLIYRDTSRPSGVPDRFSGSSSGNTATLTISGAQAGDEADYYCATSDGSGSSYQCVFGGGTQLTVT

SEQ ID NO: 33 - protein, 465 aa, synthetic construct
MDWTWRILFLVAAATGAHSQSLEESGGRLVTPGTPLTLTCTVSGFSLSSYAMGWVRQAPGKGLEWIGIIDSRGSTYYATWAKGRFTISKTSTTVDLKITSPTTEDTATYFCAREDSYYYDSFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 34 - protein, 236 aa, synthetic construct
MDWTWRILFLVAAATGAHSSFVLTQPASVQVNLGQTVSLTCTADTLSRRYASWYQQKPGQAPVLLIYRDTSRPSGVPDRFSGSSSGNTATLTISGAQAGDEADYYCATSDGSGSSYQCVFGGGTQLTVTGDQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 8 aa, synthetic construct
GFSLSSYA

SEQ ID NO: 36 - protein, 7 aa, synthetic construct
IDSRGST

SEQ ID NO: 37 - protein, 13 aa, synthetic construct
AREDSYYYDSFNL

SEQ ID NO: 38 - protein, 6 aa, synthetic construct
TLSRRY

SEQ ID NO: 39 - skipped

SEQ ID NO: 40 - protein, 13 aa, synthetic construct
ATSDGSGSSYQCV

SEQ ID NO: 41 - protein, 117 aa, synthetic construct
QSLEESGGGLFKPTDTLTLTCTASGFSLGSSYAISWVRQAPGKELEWIGTIDSGRAYYARWAKSRSTITRNTNENTVTLKMTSLTAADTATYFCARDLIPTYSQDIWGPGTLVTISS

SEQ ID NO: 42 - protein, 110 aa, synthetic construct
SFVLTQPASVQVNLGQTVSLTCTADTLSRRYASWYQQKPGQAPVLLIYRDTSRPSGVPDRFSGSSSGNTATLTISGAQAGDEADYYCATSDGSGNSYQYVFGGGTQLTVT

SEQ ID NO: 43 - protein, 466 aa, synthetic construct
MDWTWRILFLVAAATGAHSQSLEESGGGLFKPTDTLTLTCTASGFSLGSSYAISWVRQAPGKELEWIGTIDSGRAYYARWAKSRSTITRNTNENTVTLKMTSLTAADTATYFCARDLIPTYSQDIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 44 - protein, 236 aa, synthetic construct
MDWTWRILFLVAAATGAHSSFVLTQPASVQVNLGQTVSLTCTADTLSRRYASWYQQKPGQAPVLLIYRDTSRPSGVPDRFSGSSSGNTATLTISGAQAGDEADYYCATSDGSGNSYQYVFGGGTQLTVTSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 45 - protein, 8 aa, synthetic construct
GFSLGSSY

SEQ ID NO: 46 - protein, 7 aa, synthetic construct
TIDSGRA

SEQ ID NO: 47 - protein, 12 aa, synthetic construct
ARDLIPTYSQDI

SEQ ID NO: 48 - protein, 6 aa, synthetic construct
TLSRRY

SEQ ID NO: 49 - skipped

SEQ ID NO: 50 - protein, 13 aa, synthetic construct
ATSDGSGNSYQYV

SEQ ID NO: 51 - protein, 116 aa, synthetic construct
QSLGESRGRLVTPDETLTLTCTVSGIDLSSNAMSWVRQAPGEGLEWIGTINTGGRAYYASWAKGRFTISRTSTTVDLKMTSLTTEDTATYFCARERYSDGRAYDIWGPGTLVTISS

SEQ ID NO: 52 - protein, 110 aa, synthetic construct
SFVLTQPASVQVNLGQTVSLTCTADTLSRRYASWYQQKPGQAPVLLIYTDSNRPSGIPDRFSGSSSGNTATLTISGAQAGDEADYYCATDDGSGSIYQYVFGGGTQLTVT

SEQ ID NO: 53 - protein, 465 aa, synthetic construct
MDWTWRILFLVAAATGAHSQSLGESRGRLVTPDETLTLTCTVSGIDLSSNAMSWVRQAPGEGLEWIGTINTGGRAYYASWAKGRFTISRTSTTVDLKMTSLTTEDTATYFCARERYSDGRAYDIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 54 - protein, 236 aa, synthetic construct
MDWTWRILFLVAAATGAHSSFVLTQPASVQVNLGQTVSLTCTADTLSRRYASWYQQKPGQAPVLLIYTDSNRPSGIPDRFSGSSSGNTATLTISGAQAGDEADYYCATDDGSGSIYQYVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 55 - protein, 8 aa, synthetic construct
GIDLSSNA

SEQ ID NO: 56 - protein, 7 aa, synthetic construct
INTGGRA

SEQ ID NO: 57 - protein, 13 aa, synthetic construct
ARERYSDGRAYDI

SEQ ID NO: 58 - protein, 6 aa, synthetic construct
TLSRRY

SEQ ID NO: 59 - skipped

SEQ ID NO: 60 - protein, 13 aa, synthetic construct
ATDDGSGSIYQYV

SEQ ID NO: 61 - protein, 110 aa, synthetic construct
QSLEESGGRLVTPGTPLTLTCTVSGFSLSSYAMSWVRQAPGKGLEWIGAISTGGSTTYASWAKGRFTISKTSSTTVDLKMTSLTTEDTATYFCVRHFRLWGPGTLVTISS

SEQ ID NO: 62 - protein, 106 aa, synthetic construct
QVLTQTPSSVSAAVGGTVTIKCQASQSIGSALAWFQQKPGQRPKLLIYRASNLASGVPSRFKGSGSGTEFALTISGVQREDAATYYCLGGAAAVWAFGAGTNVEIK

SEQ ID NO: 63 - protein, 459 aa, synthetic construct
MDWTWRILFLVAAATGAHSQSLEESGGRLVTPGTPLTLTCTVSGFSLSSYAMSWVRQAPGKGLEWIGAISTGGSTTYASWAKGRFTISKTSSTTVDLKMTSLTTEDTATYFCVRHFRLWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 64 - protein, 232 aa, synthetic construct
MDWTWRILFLVAAATGAHSQVLTQTPSSVSAAVGGTVTIKCQASQSIGSALAWFQQKPGQRPKLLIYRASNLASGVPSRFKGSGSGTEFALTISGVQREDAATYYCLGGAAAVWAFGAGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 8 aa, synthetic construct
GFSLSSYA

SEQ ID NO: 66 - protein, 7 aa, synthetic construct
ISTGGST

SEQ ID NO: 67 - protein, 6 aa, synthetic construct
VRHFRL

SEQ ID NO: 68 - protein, 6 aa, synthetic construct
QSIGSA

SEQ ID NO: 69 - skipped

SEQ ID NO: 70 - protein, 9 aa, synthetic construct
LGGAAAVWA

SEQ ID NO: 71 - protein, 109 aa, synthetic construct
QSLEESGGRLVTPGTPLTLTCTASGFSLSSYAMSWVRQAPGKGLEWIGTMPTGGGTYYASWAKGRFTISKTSTTVDLQITSPTTEDTATYFCAKHWNIWGPGTLVTISS

SEQ ID NO: 72 - protein, 108 aa, synthetic construct
DIVLTQTPSSTSTAVGDTVTINCRASQSIGRNLAWFQQKPGQPPKLLIYRASNLASGVPSRFKGSGSRTQFTLTISGVQREDAATYYCLGSYSSSDTAFGGGTELEIL

SEQ ID NO: 73 - protein, 458 aa, synthetic construct
MDWTWRILFLVAAATGAHSQSLEESGGRLVTPGTPLTLTCTASGFSLSSYAMSWVRQAPGKGLEWIGTMPTGGGTYYASWAKGRFTISKTSTTVDLQITSPTTEDTATYFCAKHWNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 74 - protein, 234 aa, synthetic construct
MDWTWRILFLVAAATGAHSDIVLTQTPSSTSTAVGDTVTINCRASQSIGRNLAWFQQKPGQPPKLLIYRASNLASGVPSRFKGSGSRTQFTLTISGVQREDAATYYCLGSYSSSDTAFGGGTELEILRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 8 aa, synthetic construct
GFSLSSYA

SEQ ID NO: 76 - protein, 7 aa, synthetic construct
MPTGGGT

SEQ ID NO: 77 - protein, 6 aa, synthetic construct
AKHWNI

SEQ ID NO: 78 - protein, 6 aa, synthetic construct
QSIGRN

SEQ ID NO: 79 - skipped

SEQ ID NO: 80 - protein, 10 aa, synthetic construct
LGSYSSSDTA

SEQ ID NO: 81 - protein, 114 aa, synthetic construct
KQLVESGGRLVTPGTPLTLTCTVSGFSLSSYAMGWFRQAPGKGLEWIGIINSYGSTYYASWAKGRFTISKTSTTVDLKITSPTTEDTATYFCGRRIGSARFDPWGPGTLVTVSS

SEQ ID NO: 82 - protein, 110 aa, synthetic construct
SFVLTQPASVQVNLGQTVSLTCTADTLSRSYASWYQQKPGQAPVLLIYRDTRRPSGVPDRFSGSSSGNTATLTISGAQAGDEADYYCATSDGSGSSYRLVFGGGTQLTVT

SEQ ID NO: 83 - protein, 464 aa, synthetic construct
MDWTWRILFLVAAATGAHSQKQLVESGGRLVTPGTPLTLTCTVSGFSLSSYAMGWFRQAPGKGLEWIGIINSYGSTYYASWAKGRFTISKTSTTVDLKITSPTTEDTATYFCGRRIGSARFDPWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 84 - protein, 236 aa, synthetic construct
MDWTWRILFLVAAATGAHSSFVLTQPASVQVNLGQTVSLTCTADTLSRSYASWYQQKPGQAPVLLIYRDTRRPSGVPDRFSGSSSGNTATLTISGAQAGDEADYYCATSDGSGSSYRLVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 85 - protein, 8 aa, synthetic construct
GFSLSSYA

SEQ ID NO: 86 - protein, 7 aa, synthetic construct
INSYGST

SEQ ID NO: 87 - protein, 11 aa, synthetic construct
GRRIGSARFDP

SEQ ID NO: 88 - protein, 6 aa, synthetic construct
TLSRSY

SEQ ID NO: 89 - skipped

SEQ ID NO: 90 - protein, 13 aa, synthetic construct
ATSDGSGSSYRLV

SEQ ID NO: 91 - protein, 112 aa, synthetic construct
KQLVESGGRLVTPGTPLTLTCTASGFSLSSNAMSWVRQAPGEGLQWIGTISSGGSTYYASWAKGRFTISKTSTTVTLKMTSLTTEDTATYFCARGADLHRLWGPGTLVTVSS

SEQ ID NO: 92 - protein, 110 aa, synthetic construct
SFVLTQPASVQVNLGQTVSLTCTGKPLSRSFASWYQQKPGQAPVLVIYRDTSRPSGVPDRFSGSSSGNTATLTISGAQAGDEADYYCATSDGSGSSYQYVFGGGTQLTVT

SEQ ID NO: 93 - protein, 462 aa, synthetic construct
MDWTWRILFLVAAATGAHSQKQLVESGGRLVTPGTPLTLTCTASGFSLSSNAMSWVRQAPGEGLQWIGTISSGGSTYYASWAKGRFTISKTSTTVTLKMTSLTTEDTATYFCARGADLHRLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 94 - protein, 236 aa, synthetic construct
MDWTWRILFLVAAATGAHSSFVLTQPASVQVNLGQTVSLTCTGKPLSRSFASWYQQKPGQAPVLVIYRDTSRPSGVPDRFSGSSSGNTATLTISGAQAGDEADYYCATSDGSGSSYQYVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 95 - protein, 8 aa, synthetic construct
GFSLSSNA

SEQ ID NO: 96 - protein, 7 aa, synthetic construct
ISSGGST

SEQ ID NO: 97 - protein, 9 aa, synthetic construct
ARGADLHRL

SEQ ID NO: 98 - protein, 6 aa, synthetic construct
PLSRSF

SEQ ID NO: 99 - skipped

SEQ ID NO: 100 - protein, 13 aa, synthetic construct
ATSDGSGSSYQYV

SEQ ID NO: 101 - protein, 118 aa, synthetic construct
QSVKESEGGLFKPTDTLTLTCTASGFTVSTNAVTWVRQAPGNGLEYIGTIGRSGSAYFASWAKRRSTLTRDINLNTVTLKMTSLTAADTATYFCVRGSPGYRTAISIWGPGTLVTISS

SEQ ID NO: 102 - protein, 107 aa, synthetic construct
AVMTQTPSSTSAAVEGTVTINCQASQSISNRLSWFQQKPGQPPKLLIYQASTLASGVPSRFKGSGSGTEFTLTISGVQREDAATYYCLGSYSSSDASFGGGTKLEIK

SEQ ID NO: 103 - protein, 467 aa, synthetic construct
MDWTWRILFLVAAATGAHSQSVKESEGGLFKPTDTLTLTCTASGFTVSTNAVTWVRQAPGNGLEYIGTIGRSGSAYFASWAKRRSTLTRDINLNTVTLKMTSLTAADTATYFCVRGSPGYRTAISIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 104 - protein, 233 aa, synthetic construct
MDWTWRILFLVAAATGAHSAVMTQTPSSTSAAVEGTVTINCQASQSISNRLSWFQQKPGQPPKLLIYQASTLASGVPSRFKGSGSGTEFTLTISGVQREDAATYYCLGSYSSSDASFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 8 aa, synthetic construct
GFTVSTNA

SEQ ID NO: 106 - protein, 7 aa, synthetic construct
IGRSGSA

SEQ ID NO: 107 - protein, 13 aa, synthetic construct
VRGSPGYRTAISI

SEQ ID NO: 108 - protein, 6 aa, synthetic construct
QSISNR

SEQ ID NO: 109 - skipped

SEQ ID NO: 110 - protein, 10 aa, synthetic construct
LGSYSSSDAS

SEQ ID NO: 111 - protein, 114 aa, synthetic construct
KQLVESGGGLVTPGGTLTLTCTVSGFSLSSNGVSWVRQAPGKGLEYIGLIRSIGSTYYATWAKGRFTISKTSTTVDLKITSPTTEDTATYFCARGGYRNGFKLWGPGTLVTVSS

SEQ ID NO: 112 - protein, 111 aa, synthetic construct
PVLTQSPSVSAALGASAKLTCTLSSAHKTYYIEWYQQQHPGKAPRYLMQLQSDGSYTKGTGVPDRFSGSSSGADRYLIISSVQAEDEADYICGVIDSAVYVFGGGTQLTVT

SEQ ID NO: 113 - protein, 464 aa, synthetic construct
MDWTWRILFLVAAATGAHSQKQLVESGGGLVTPGGTLTLTCTVSGFSLSSNGVSWVRQAPGKGLEYIGLIRSIGSTYYATWAKGRFTISKTSTTVDLKITSPTTEDTATYFCARGGYRNGFKLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 114 - protein, 238 aa, synthetic construct
MDWTWRILFLVAAATGAHSQPVLTQSPSVSAALGASAKLTCTLSSAHKTYYIEWYQQQHPGKAPRYLMQLQSDGSYTKGTGVPDRFSGSSSGADRYLIISSVQAEDEADYICGVIDSAVYVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 115 - protein, 8 aa, synthetic construct
GFSLSSNG

SEQ ID NO: 116 - protein, 7 aa, synthetic construct
IRSIGST

SEQ ID NO: 117 - protein, 11 aa, synthetic construct
ARGGYRNGFKL

SEQ ID NO: 118 - protein, 7 aa, synthetic construct
AHKTYYI

SEQ ID NO: 119 - protein, 7 aa, synthetic construct
LQSDGSY

SEQ ID NO: 120 - protein, 9 aa, synthetic construct
GVIDSAVYV

SEQ ID NO: 121 - protein, 115 aa, synthetic construct
KQLVESGGRLVTPGTPLTLTCTVTGIDLSSHGMSWVRQAPGKGLEWIGVIYPSGSTWYASWARGRFTISKTSTTVDLKMTSLTTEDTATHFCARGDVGGADNNIWGPGTLVTVSS

SEQ ID NO: 122 - protein, 110 aa, synthetic construct
SFVLTQPASVQVDLGQTVSLTCTAETLSRSYASWYQQKPGQAPVLLIYRDTSRPSGVPDRFSGSSSGNTATLTISGAQAGDEADYYCATSDGSGSSYQYVFGGGTQLTVT

SEQ ID NO: 123 - protein, 465 aa, synthetic construct
MDWTWRILFLVAAATGAHSQKQLVESGGRLVTPGTPLTLTCTVTGIDLSSHGMSWVRQAPGKGLEWIGVIYPSGSTWYASWARGRFTISKTSTTVDLKMTSLTTEDTATHFCARGDVGGADNNIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 124 - protein, 236 aa, synthetic construct
MDWTWRILFLVAAATGAHSSFVLTQPASVQVDLGQTVSLTCTAETLSRSYASWYQQKPGQAPVLLIYRDTSRPSGVPDRFSGSSSGNTATLTISGAQAGDEADYYCATSDGSGSSYQYVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 125 - protein, 8 aa, synthetic construct
GIDLSSHG

SEQ ID NO: 126 - protein, 7 aa, synthetic construct
IYPSGST

SEQ ID NO: 127 - protein, 12 aa, synthetic construct
ARGDVGGADNNI

SEQ ID NO: 128 - protein, 6 aa, synthetic construct
TLSRSY

SEQ ID NO: 129 - skipped

SEQ ID NO: 130 - protein, 13 aa, synthetic construct
ATSDGSGSSYQYV

SEQ ID NO: 131 - protein, 115 aa, synthetic construct
QEQLVESGGRLVTPGTPLTLTCKASGFSLGKYDMSWVRQAPGKGLEWIGTIYAGSGSTWYASWAKGRFTISKTSTTVDLKMTSLTTEDTATYFCARDFLGVNSIWGPGTLVTISS

SEQ ID NO: 132 - protein, 110 aa, synthetic construct
SFVLTQPASVQVNLGQTVSLTCTADTLSRSYASWYQQKPDQAPVLLIHRDTSRPSGVPDRFSGSNSGNTATLTISGAQAGDEADYYCATSDGSGSNFHFVFGGGTQLTVT

SEQ ID NO: 133 - protein, 464 aa, synthetic construct
MDWTWRILFLVAAATGAHSQEQLVESGGRLVTPGTPLTLTCKASGFSLGKYDMSWVRQAPGKGLEWIGTIYAGSGSTWYASWAKGRFTISKTSTTVDLKMTSLTTEDTATYFCARDFLGVNSIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 134 - protein, 236 aa, synthetic construct
MDWTWRILFLVAAATGAHSSFVLTQPASVQVNLGQTVSLTCTADTLSRSYASWYQQKPDQAPVLLIHRDTSRPSGVPDRFSGSNSGNTATLTISGAQAGDEADYYCATSDGSGSNFHFVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 135 - protein, 8 aa, synthetic construct
GFSLGKYD

SEQ ID NO: 136 - protein, 8 aa, synthetic construct
IYAGSGST

SEQ ID NO: 137 - protein, 10 aa, synthetic construct
ARDFLGVNSI

SEQ ID NO: 138 - protein, 6 aa, synthetic construct
TLSRSY

SEQ ID NO: 139 - skipped

SEQ ID NO: 140 - protein, 13 aa, synthetic construct
ATSDGSGSNFHFV

SEQ ID NO: 141 - protein, 119 aa, synthetic construct
KQLVESGGGLVQPGGSLTLTCTVSGFSLNKYYMGWFRQAPGKGLEWIGVISRSGDTYYSSWAKGRFTISKASTTVDLRITSPTTEDTATYFCARDASSPGFHGDRLDLWGQGTLVTVSS

SEQ ID NO: 142 - protein, 110 aa, synthetic construct
SFVLTQPASVQVNLGQTVSLTCTADTLSRSYASWYQQKPGQAPVLLIYRDTSRPSGVPDRFSGSSSGNTATLTISGAQAGDEADYYCATSDGIGSNYLWVFGGGTQLTVT

SEQ ID NO: 143 - protein, 469 aa, synthetic construct
MDWTWRILFLVAAATGAHSQKQLVESGGGLVQPGGSLTLTCTVSGFSLNKYYMGWFRQAPGKGLEWIGVISRSGDTYYSSWAKGRFTISKASTTVDLRITSPTTEDTATYFCARDASSPGFHGDRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 144 - protein, 236 aa, synthetic construct
MDWTWRILFLVAAATGAHSSFVLTQPASVQVNLGQTVSLTCTADTLSRSYASWYQQKPGQAPVLLIYRDTSRPSGVPDRFSGSSSGNTATLTISGAQAGDEADYYCATSDGIGSNYLWVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 145 - protein, 8 aa, synthetic construct
GFSLNKYY

SEQ ID NO: 146 - protein, 7 aa, synthetic construct
ISRSGDT

SEQ ID NO: 147 - protein, 16 aa, synthetic construct
ARDASSPGFHGDRLDL

SEQ ID NO: 148 - protein, 6 aa, synthetic construct
TLSRSY

SEQ ID NO: 149 - skipped

SEQ ID NO: 150 - protein, 13 aa, synthetic construct
ATSDGIGSNYLWV

SEQ ID NO: 151 - DNA, 19 bp, synthetic construct
  source 1..19: mol_type = other DNA
ggcccaggcaccctggtca

SEQ ID NO: 152 - DNA, 19 bp, synthetic construct
  source 1..19: mol_type = other DNA
ggccagggcaccctggtca

SEQ ID NO: 153 - DNA, 19 bp, synthetic construct
  source 1..19: mol_type = other DNA
ggcccagggaccctcgtca

SEQ ID NO: 154 - DNA, 19 bp, synthetic construct
  source 1..19: mol_type = other DNA
ggcccsggcaccctggtca

SEQ ID NO: 155 - DNA, 19 bp, synthetic construct
  source 1..19: mol_type = other DNA
ggtccaggcaccctggtca

SEQ ID NO: 156 - DNA, 33 bp, synthetic construct
  source 1..33: mol_type = other DNA
acgacgctcttccgatctgcccttggtggaggc

SEQ ID NO: 157 - DNA, 33 bp, synthetic construct
  source 1..33: mol_type = other DNA
cgtgtgctcttccgatctcaaccagccatggcc

SEQ ID NO: 158 - DNA, 58 bp, synthetic construct
  source 1..58: mol_type = other DNA
aatgatacggcgaccaccgagatctacactctttccctacacgacgctcttccgatct

SEQ ID NO: 159 - DNA, 64 bp, synthetic construct
  source 1..64: mol_type = other DNA
caagcagaagacggcatacgagatcgtgatgtgactggagttcagacgtgtgctcttccgatct

SEQ ID NO: 160 - DNA, 64 bp, synthetic construct
  source 1..64: mol_type = other DNA
caagcagaagacggcatacgagatacatcggtgactggagttcagacgtgtgctcttccgatct

SEQ ID NO: 161 - DNA, 64 bp, synthetic construct
  source 1..64: mol_type = other DNA
caagcagaagacggcatacgagatgcctaagtgactggagttcagacgtgtgctcttccgatct

SEQ ID NO: 162 - DNA, 64 bp, synthetic construct
  source 1..64: mol_type = other DNA
caagcagaagacggcatacgagattggtcagtgactggagttcagacgtgtgctcttccgatct

SEQ ID NO: 163 - DNA, 64 bp, synthetic construct
  source 1..64: mol_type = other DNA
caagcagaagacggcatacgagatcactgtgtgactggagttcagacgtgtgctcttccgatct

SEQ ID NO: 164 - DNA, 64 bp, synthetic construct
  source 1..64: mol_type = other DNA
caagcagaagacggcatacgagatattggcgtgactggagttcagacgtgtgctcttccgatct

SEQ ID NO: 165 - DNA, 64 bp, synthetic construct
  source 1..64: mol_type = other DNA
caagcagaagacggcatacgagatgatctggtgactggagttcagacgtgtgctcttccgatct

SEQ ID NO: 166 - DNA, 64 bp, synthetic construct
  source 1..64: mol_type = other DNA
caagcagaagacggcatacgagattcaagtgtgactggagttcagacgtgtgctcttccgatct

SEQ ID NO: 167 - DNA, 64 bp, synthetic construct
  source 1..64: mol_type = other DNA
caagcagaagacggcatacgagatctgatcgtgactggagttcagacgtgtgctcttccgatct

SEQ ID NO: 168 - DNA, 64 bp, synthetic construct
  source 1..64: mol_type = other DNA
caagcagaagacggcatacgagataagctagtgactggagttcagacgtgtgctcttccgatct

SEQ ID NO: 169 - DNA, 64 bp, synthetic construct
  source 1..64: mol_type = other DNA
caagcagaagacggcatacgagatgtagccgtgactggagttcagacgtgtgctcttccgatct

SEQ ID NO: 170 - DNA, 64 bp, synthetic construct
  source 1..64: mol_type = other DNA
caagcagaagacggcatacgagattacaaggtgactggagttcagacgtgtgctcttccgatct

SEQ ID NO: 171 - protein, 4 aa, synthetic construct
TYFC

SEQ ID NO: 172 - protein, 19 aa, synthetic construct
MDWTWRILFLVAAATGAHS

SEQ ID NO: 173 - protein, 4 aa, synthetic construct
GGGS

SEQ ID NO: 174 - protein, 16 aa, synthetic construct
SEPKSSDKTHTCPPCP